(19) United States
(12) Patent Application Publication
(10) Pub. No.: US 2021/0164048 A1
(43) Pub. Date: Jun. 3, 2021

(54) A NON-INVASIVE PRENATAL TEST WITH ACCURATE FETAL FRACTION MEASUREMENT

(71) Applicant: SINGLERA GENOMICS, INC., La Jolla, CA (US)

(72) Inventors: Yuan GAO, La Jolla, CA (US); Rui LIU, La Jolla, CA (US); Christopher HARTL, La Jolla, CA (US); Bin XIE, La Jolla, CA (US); Jingyi LU, La Jolla, CA (US)

(73) Assignee: SINGLERA GENOMICS, INC., La Jolla, CA (US)

(21) Appl. No.: 17/265,803

(22) PCT Filed: Aug. 1, 2019
(86) PCT No.: PCT/US2019/044721

§ 371 Date: Feb. 3, 2021

Related U.S. Application Data
(60) Provisional application No. 62/715,585, filed on Aug. 7, 2018.

(51) Int. Cl. C12Q 1/6879; C12Q 1/6827

(52) U.S. Cl. CPC C12Q 1/6879; C12Q 2600/156; C12Q 1/6827

(57) ABSTRACT

The present disclosure relates to methods for non-invasive prenatal testing (NIPT) using semm from a maternal blood sample taken during pregnancy. The methods provide efficient access to genetic information about the fetus, including gender, fetal DNA fraction, paternity, and possible genetic abnormalities. This approach is referred to herein as Afisawa, and makes NIPT genetic testing more efficient and cost effective than previous methods.

### RELATED APPLICATION

The present application claims priority to U.S. provisional patent application No. 62/715,585, filed on Aug. 7, 2018, the disclosure of which is incorporated by reference in its entirety for all purposes.

### TECHNICAL FIELD

The present disclosure relates to a non-invasive prenatal screening assay for analyzing fetal DNA. The methods can be used for testing of chromosomal aneuploidy, measuring fetal DNA fraction, identifying fetal sex, prenatal paternity test, and other genetic testing, using plasma-derived cell-free DNA obtained from a pregnant woman.

### BACKGROUND

In the following discussion, certain articles and methods are described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Chromosomal aneuploidy—the presence of an abnormal number of chromosomes within a person's cells—is a germline genetic defect which nearly always gives rise to severe developmental deficits. The presence of a third copy of chromosome 21 (Trisomy 21, or Down Syndrome) is the most common of these genetic diseases, occurring between 0.1%-0.15% of pregnancies. Other observed chromosomal abnormalities include Edward syndrome (trisomy 18), Patau syndrome (trisomy 13), Turner syndrome (45, X), Klinefelter syndrome (47, XXY), 47, XYY, and trisomy X (47, XXX). These diseases, while rare, occur frequently enough and with sufficient consequences to merit widespread screening.

Fetal aneuploidy diagnostics such as amniocentesis and chorionic villus sampling are invasive, thus carry the risk of pregnancy loss. This makes invasive diagnostic approaches unsuitable for population-wide screening. The presence of fetal cell-free DNA (cfDNA) in the plasma of circulating maternal blood enables a non-invasive genetics-based approach [1, 2, 3]. This screening identifies a population significantly enriched for true cases of chromosomal aneuploidy, on which the more invasive diagnostic procedures can be applied as needed to provide an aggregate reduction of risk.

Genetics-based NIPT methods have a long history, with initial findings of fetal cfDNA dating to 1997 [2], and academic demonstrations of NIPT screening throughout 2000-2010 [4,5]; demonstrating that genetics-based screening outperformed visual screening based on ultrasounds [6-8]. The first commercial NIPT tests (Sequenom's MaterniT21 in 2011, Verinata Health's Verifi in 2012) each used shotgun whole-genome sequencing. Ariosa's Harmony pioneered a capture-based targeted sequencing approach to maximize information coming from the most common abnormalities (T21, T18, T13) [9-11]. Natera's Panorama targets a panel of single-nucleotide polymorphisms to assess both fetal fraction and aneuploidy[12, 13].While these are all US tests, these and similar NIPT methods are also available in other countries. However, there are challenges that limit the widespread global adoption of cfDNA-based NIPT. One challenge is the relatively high cost of NIPT. Sample collection, sample transportation, sample processing, the detection method being used, and sequencing costs all contribute to the high cost of NIPT. Another challenge is the low fetal DNA concentration in some maternal plasma samples. False negative results arise when the fetal DNA concentration is too low [14], thus the ACMG recommends that laboratories include a clearly visible fetal fraction on NIPT reports[15]. Several bioinformatics approaches have been developed to estimate fetal DNA fraction in NIPT[16]. Those approaches have various limitations such as lower accuracy, not being applicable for female pregnancy, and/or high cost.

There is a need for improved methods to use NIPT to identify fetal genetic characteristics. The present disclosure addresses this and other related needs by utilizing several new approaches in DNA target selection and DNA capture technology.

### BRIEF SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

The methods described herein are referred to as Afisawa, which is a target based non-invasive prenatal test (NIPT) assay with an amplification of selected SNP/SNPs rich human chromosome regions. Afisawa overcomes two limitations present in other whole genome shotgun sequencing based non-invasive prenatal screening (NIPT) assays. First, it only requires as little as 0.5 to 1 million raw sequencing reads, which means it can be performed cost-effectively, using any of a number of desktop sequencers. Second, it can simultaneously and accurately measure fetal fractions as low as 3%, thus reducing the number of inconclusive results and the rate of false negatives.

Afisawa is a novel NIPT assay for testing of chromosomal aneuploidy, measuring the fetal DNA fraction, and distinguishing male from female pregnancy using cfDNA extracted from plasma of a pregnant woman. The overall process is summarized in FIG. 1. Blood from a pregnant woman is collected in a suitable vessel such as a Streck Cell free DNA tube or EDTA tube. The whole assay work flow takes three to five working days as shown, and includes: Plasma separation, cfDNA extraction, Afisawa NIPT library preparation, DNA sequencing, and generation of a report.

The Afisawa assay uses a plurality of probes, typically ˜1800 to ˜5000 single stranded DNA connector inversion probes, to capture selected genomic DNA regions on chromosomes 1, 2, 3, 4, 9, 13, 15, 18, 19, 21, 22, X, and Y. The selected capture regions are enriched, indexed, and sequenced using next-generation sequencing technology such as the Illumina or IonTorrent families of sequencers. The assay requires a little as 3 ng to 6 ng cfDNA from a pregnant woman, which is sequenced to a total depth of half million to one million reads. The resulting DNA measurements are used to calculate the probability of chromosomal aneuploidy (T21, T18, T13), fetal DNA fraction, and fetal sex, using algorithms described herein. Using 6 ng of cfDNA, Afisawa is able to measure accurately fetal fractions as low as 3%, identify sex chromosomes, and detect chromosomal aneuploidies. The Afisawa assay can be easily expanded to detect chromosomal aneuploidies other than T21, T18, or T13, as well as micro deletions and single-gene disorders with an addition of respective single stranded DNA connector inversion probes. The Afisawa technology can also be utilized for non-invasive prenatal paternity tests.

A critical aspect of Afisawa is target selection. Selected genomic DNA regions containing one or more SNPs on chromosomes 1, 2, 3, 4, 9, 13, 15, 18, 19, 21, 22, X, and Y are targeted. Examples of selected targets are illustrated in FIG. 2A and FIG. 2B. Both Watson and Crick strands of DNA are targeted. About 1800-5000 probes are selected from ˜14000 initially designed probes. Nearly 5000 target regions with SNP information are summarized in Table 1 (the Table in FIG. 10). By combining many probes for each target of interest, Afisawa can simultaneously detect chromosome aneuploidy and measure fetal fraction with a small number of raw sequencing reads.

Another key technical aspect is the usage of connector inversion probe capture technology on cell-free DNA (cfDNA) of pregnant women for NIPT. Connector Inversion Probe is a powerful single stranded DNA probe based multiplex DNA amplification system for numerous scientific applications [17, 18]. Connector inversion probes have been widely used to capture selected targets using genomic DNA as input template [19-22]. Capture of targets on cfDNA is challenging due to the small size of cfDNA fragments. To maximize our probe selections, both Watson and Crick strands of DNA are targeted. The gap between two gene specific arms is optimized to be around 40 bp. Furthermore, the whole work flow of Afisawa NIPT assay from target capture to the generation of indexed, enriched DNA library is simplified. A bioinformatic pipeline is also developed to identify sex chromosomes, detect chromosomal aneuploidies, and measure fetal fraction using the same sequencing data set.

In summary, Afisawa is a novel non-invasive prenatal screening assay for testing of chromosomal aneuploidy, measuring fetal DNA fraction, and identifying fetal sex using plasma-derived cell-free DNA obtained from pregnant women. Afisawa NIPT assay is designed to be performed with a desktop sequencer such as Illumina Miseq, Miniseq or Lifetech S5 due to its requirement of a relatively small number of sequencing reads. The Afisawa is robust and cost effective, thus it could make NIPT assays more affordable and accessible for pregnant women.

Afisawa assay utilizes around 1800 to 5000 single stranded DNA connector inversion probes to simultaneously capture selected human genomic DNA regions on chromosome 21, 18, 13, X, Y, and some other autosomal chromosomes. The selected capture regions are further enriched and indexed for the individual sample and massively sequenced on either Illumina sequencer (Miseq, miniseq, or Nextseq et. al) or Ion Torrent sequencer (S5, ion proton et. al). The input materials could be either a mixture of reference genomic DNA fragments or cfDNA of pregnant women. As low as 6 ng cfDNA from less than 2 ml plasma of a pregnant woman is sufficient for SGI Afisawa NIPT assay. Only around half million raw reads are needed for one sample test. The human mapped reads are used to calculate the probability of chromosomal aneuploidy (T21, T18, T13), fetal DNA fraction, and male/female fetal gender using an algorithm that is explained herein. Our results demonstrated that Afisawa is able to measure the fetal fraction as low as 3% with 6 ng input either mixture of reference genomic DNA fragments or cfDNA of pregnant women. Afisawa is able to detect as low as 10 copies of Y chromosome DNA from a mixture of reference genomic DNA fragments. Our preliminary data showed that Afisawa is capable of detecting T21 in both mixtures of genomic DNA and cfDNA from pregnant women. Afisawa could be also potentially used as a paternity test.

In one aspect, the invention provides a plurality of polynucleotides, wherein each polynucleotide comprises:
a first target-specific domain and a second target-specific domain configured to bind to a first target sequence and a second target sequence, respectively, of a nucleic acid target, and a unique molecule identifier (UMI) and a linker between the first and second target-specific domains,
   wherein the first and second target-specific domains are configured to be connected to each other such that the polynucleotide forms a circle, optionally after a polymerase-mediated extension of the first or second target-specific domain, and
   wherein the nucleic acid target comprises a polymorphic nucleotide within the first target sequence and/or the second target sequence, or between the first and second target sequences.

In some embodiments of this aspect, each of the polynucleotides of the plurality of polynucleotides used in this method is a polynucleotide probe that comprises a first target domain and a second target domain connected by a linker that comprises a unique molecular identifier (UMI). The UMI is about 5-15 nucleotides in length, typically 5-10, and preferably 6-8 nucleotides in length. Upon polymerase-mediated extension in the presence of a sample containing the targets of the first and second target-specific domains, these polynucleotides form a circle, i.e., the product of polymerase-mediated extension is a circular polynucleotide, and it contains the linker, UMI, and first and second target-specific domain sequences. Subsequent analysis of the circular polynucleotides (the presence or absence and/or amount of such circular polynucleotides as well as their sequences) permits the user to determine fetal fraction of the DNA sample being tested, and to identify genetic information associated with the nucleic acid target as further described herein. Typically, the circular polynucleotides from a sample that has undergone polymerase-mediated extension are further enriched or amplified by PCR before analysis. This produces sufficient polynucleotide for sequence analysis. Suitable polynucleotides and polynucleotide probes for use in this method are disclosed herein; and based on the information provided herein, a skilled person could readily design and construct suitable polynucleotide probes for use in a variety of applications of these methods.

In a second aspect, the invention provides a method for analyzing a fetal genetic information, e.g., fetal fraction, a chromosome abnormality such as trisomy, sex determination and/or prenatal paternity test, comprising:
a) contacting a sample from a female subject with the plurality of polynucleotides according to the preceding aspect; and
b) wherein nucleic acid sequence information of the sample is obtained, which indicates a fetal genetic information.

In another aspect, the invention provides a kit for analyzing a fetal genetic information, e.g., fetal fraction, a chromosome abnormality such as trisomy, sex determination and/or prenatal paternity test, comprising a plurality of polynucleotides according to the first aspect above.

These and other aspects of the invention are more fully described in the detailed description below.

### BRIEF DESCRIPTION OF THE DRAWINGS

FIG. 1. Overview of SGI Afisawa NIPT assay.

FIG. 2. Afisawa Target selection. Selected genomic DNA regions containing one or more SNPs on chromosomes 1, 2, 3, 4, 9, 13, 15, 18, 19, 21, 22, X, and Y are targeted. Both Watson and Crick strands of DNA are targeted. ˜5000 probes are selected from ˜14000 initially designed probes. ˜5000 targeted regions with SNP information are summarized in Table 1 (the Table in FIG. 10).

FIG. 3. Single stranded DNA connector inversion probes that can be used to capture selected regions of human genomic DNA. FIG. 3 consists of FIG. 3A, FIG. 3B, and FIG. 3C.

FIG. 4 consists of FIGS. 4A and 4B. 4A. Relationships of four individuals whose genomic DNAs being used in our studies; 4B. Descriptions of fragmented genomic DNA mixtures being used in our studies.

FIG. 5 consists of FIGS. 5A, 5B, 5C, and 5D. Sample of Data analysis and Sample output of Afisawa NIPT assay.

FIG. 6. Detection of male/female pregnancy by Afisawa, FIG. 6 consists of FIG. 6A and FIG. 6B.

FIG. 7. Measurement of fetal fraction by Afisawa. FIG. 7 consists of FIGS. 7A-7D.

FIG. 8. Testing of chromosomal aneuploidy (T21, T18, or T13) by Afisawa; FIG. 8 consists of FIG. 8A, FIG. 8B, and FIG. 8C.

FIG. 9, consisting of FIG. 9A and FIG. 9B. Accurate fetal fraction estimated using only the probes on Chr. 1, 4, 22.

FIG. 10. Table 1. List of ˜5000 targets for Afisawa NIPT assays.

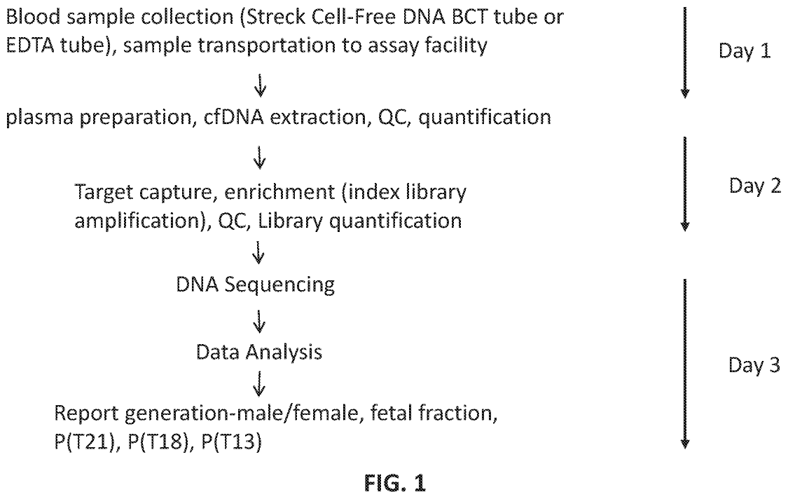

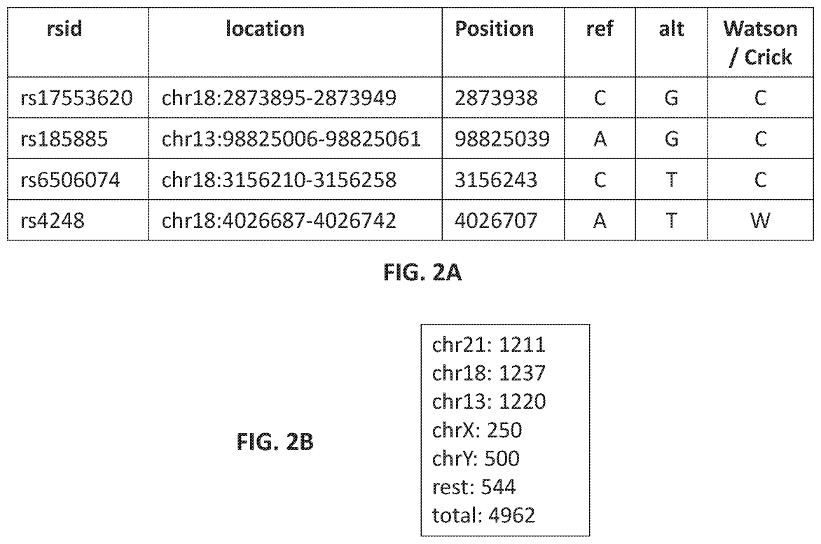

### DETAILED DESCRIPTION

Numerous specific details are set forth in the following description in order to provide a thorough understanding of the methods and other aspects of the invention. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied alone, or in some combination, to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All publications, including patent documents, scientific articles and databases, referred to in this application are incorporated by reference in their entireties for all purposes to the same extent as if each individual publication were individually incorporated by reference. Citation of the publications or documents is not intended as an admission that any of them is pertinent prior art, nor does it constitute any admission as to the contents or date of these publications or documents.

All headings are for the convenience of the reader and should not be used to limit the meaning of the text that follows the heading, unless so specified.

The practice of the provided embodiments will employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polypeptide and protein synthesis and modification, polynucleotide synthesis and modification, polymer array synthesis, hybridization and ligation of polynucleotides, detection of hybridization, and nucleotide sequencing. Specific illustrations of suitable techniques can be had by reference to the examples herein. However, other equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, Gabriel, Stephens, Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Ausubel et al. eds., Current Protocols in Molecular Biology (1987); T. Brown ed., Essential Molecular Biology (1991), IRL Press; Goeddel ed., Gene Expression Technology (1991), Academic Press; A. Bothwell et al. eds., Methods for Cloning and Analysis of Eukaryotic Genes (1990), Bartlett Publ.; M. Kriegler, Gene Transfer and Expression (1990), Stockton Press; R. Wu et al. eds., Recombinant DNA Methodology (1989), Academic Press; M. McPherson et al., PCR: A Practical Approach (1991), IRL Press at Oxford University Press; Stryer, Biochemistry (4th Ed.) (1995), W. H. Freeman, New York N.Y.; Gait, Oligonucleotide Synthesis: A Practical Approach (2002), IRL Press, London; Nelson and Cox, Lehninger, Principles of Biochemistry (2000) 3rd Ed., W. H. Freeman Pub., New York, N.Y.; Berg, et al., Biochemistry (2002) 5th Ed., W. H. Freeman Pub., New York, N.Y., all of which are herein incorporated in their entireties by reference for all purposes.

### A. Definitions

Unless defined otherwise, all technical and scientific terms used herein have the same meaning as is commonly understood by one of ordinary skill in the art to which the present disclosure belongs. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, the singular forms “a,” “an,” and “the” include the plural reference unless the context clearly dictates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Reference to “about” a value or parameter herein includes (and describes) variations that are directed to that value or parameter per se. For example, description referring to “about X” includes description of “X”. Additionally, use of “about” preceding any series of numbers includes “about” each of the recited numbers in that series. For example, description referring to “about X, Y, or Z” is intended to describe “about X, about Y, or about Z.”

The term “average” as used herein refers to either a mean or a median, or any value used to approximate the mean or the median, unless the context clearly indicates otherwise.

A “subject” as used herein refers to an organism, or a part or component of the organism, to which the provided compositions, methods, kits, devices, and systems can be administered or applied. For example, the subject can be a mammal or a cell, a tissue, an organ, or a part of the mammal. As used herein, “mammal” refers to any of the mammalian class of species, preferably human (including humans, human subjects, or human patients). Mammals include, but are not limited to, farm animals, sport animals, pets, primates, horses, dogs, cats, and rodents such as mice and rats. Typically a subject is a mammal; preferably a subject is a human.

As used herein the term “sample” refers to anything which may contain a target molecule for which analysis is desired, including a biological sample. As used herein, a “biological sample” can refer to any sample obtained from a living or viral (or prion) source or other source of macromolecules and biomolecules, and includes any cell type or tissue of a subject from which nucleic acid, protein and/or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine, sweat, semen, stool, sputum, tears, mucus, amniotic fluid or the like, an effusion, a bone marrow sample, ascitic fluid, pelvic wash fluid, pleural fluid, spinal fluid, lymph, ocular fluid, extract of nasal, throat or genital swab, cell suspension from digested tissue, or extract of fecal material, and tissue and organ samples from animals and plants and processed samples derived therefrom.

The terms “polynucleotide,” “oligonucleotide,” “nucleic acid” and “nucleic acid molecule” are used interchangeably herein to refer to a polymeric form of nucleotides of any length, and comprise ribonucleotides, deoxyribonucleotides, and analogs or mixtures thereof. The terms include triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the terms “polynucleotide,” “oligonucleotide,” “nucleic acid,” and “nucleic acid molecule” include polydeoxyribonucleotides (containing 2-deoxy-D-ribose), polyribonucleotides (containing D-ribose), including tRNA, rRNA, hRNA, and mRNA, whether spliced or unspliced, any other type of polynucleotide which is an N- or C-glycoside of a purine or pyrimidine base, and other polymers containing nonnucleotidic backbones, for example, polyamide (e.g., peptide nucleic acids (“PNAs”)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. Thus, these terms include, for example, 3′-deoxy-2′,5′-DNA, oligodeoxyribonucleotide N3′ to P5′ phosphoramidates, 2′-O-alkyl-substituted RNA, hybrids between DNA and RNA or between PNAs and DNA or RNA, and also include known types of modifications, for example, labels, alkylation, “caps,” substitution of one or more of the nucleotides with an analog, inter-nucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters), those containing pendant moieties, such as, for example, proteins (including enzymes (e.g. nucleases), toxins, antibodies, signal peptides, poly-L-lysine, etc.), those with intercalators (e.g., acridine, psoralen, etc.), those containing chelates (of, e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. A nucleic acid generally will contain phosphodiester bonds, although in some cases nucleic acid analogs may be included that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA:DNA hybrids can exhibit higher stability in some environments. The terms “polynucleotide,” “oligonucleotide,” “nucleic acid” and “nucleic acid molecule” can comprise any suitable length, such as at least 5, 6, 7, 8, 9, 10, 20, 30, 40, 50, 100, 200, 300, 400, 500, or 1,000, or more than 1,000 nucleotides.

It will be appreciated that, as used herein, the terms “nucleoside” and “nucleotide” include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides can also include modifications on the sugar moiety, e.g., wherein one or more of the hydroxyl groups are replaced with halogen, aliphatic groups, or are functionalized as ethers, amines, or the like. The term “nucleotidic unit” is intended to encompass nucleosides and nucleotides. In preferred embodiments, the nucleoside or nucleotide is selected from the natural moieties comprised in DNA or RNA.

The terms “complementary” and “substantially complementary” include the hybridization or base pairing or the formation of a duplex between nucleotides or nucleic acids, for instance, between the two strands of a double-stranded DNA molecule or between an oligonucleotide primer and a primer binding site on a single-stranded nucleic acid. Complementary nucleotides are, generally, A and T (or A and U), or C and G. Two single-stranded RNA or DNA molecules are said to be substantially complementary when the nucleotides of one strand, optimally aligned and compared and with appropriate nucleotide insertions or deletions, pair with at least about 80% of the other strand, usually at least about 90% to about 95%, and even about 98% to about 100%. In one aspect, two complementary sequences of nucleotides are capable of hybridizing, preferably with less than 25%, more preferably with less than 15%, even more preferably with less than 5%, most preferably with no mismatches between opposed nucleotides. Preferably the two molecules will hybridize under conditions of high stringency.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers known in the art. Hybridization temperatures can be as low as 5° C., but are typically greater than 22° C., and more typically greater than about 30° C., and typically in excess of 37° C. Hybridizations are often performed under stringent conditions, i.e., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5 ×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, EDTA) contains 3 M sodium chloride, 0.2 M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a polymerase, for example, a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically. As used herein, ligations are usually carried out enzymatically to form a phosphodiester linkage between a 5′ carbon terminal nucleotide of one oligonucleotide with a 3′ carbon of another nucleotide.

“Amplification,” as used herein, generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

“Sequence determination” and the like include determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid.

The term “Sequencing,” “High throughput sequencing,” or “next generation sequencing” includes sequence determination using methods that determine many (typically thousands to billions) of nucleic acid sequences in an intrinsically parallel manner, i.e. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, CT); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeg™ technology by Illumina, Inc., San Diego, Calif.; HeliScope™ by Helicos Biosciences Corporation, Cambridge, Mass.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edu/cgi-bin/hgGateway) or the NCBI dbSNP website (ncbi.nlm nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations.

It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome. Sequencing technologies are available for SNP sequencing, such as the BeadArray platform (GOLDENGATE™ assay) (Illumina, Inc., San Diego, Calif.) (see Fan, et al., Cold Spring Symp. Quant. Biol., 68:69-78 (2003)), may be employed.

“Multiplexing” or “multiplex assay” herein may refer to an assay or other analytical method in which the presence and/or amount of multiple targets, e.g., multiple nucleic acid sequences, can be assayed simultaneously by using more than one markers, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, “disease or disorder” refers to a pathological condition in an organism resulting from, e.g., infection or genetic defect, and characterized by identifiable symptoms.

### B. Overview

The following enumerated embodiments are representative of certain facets of the invention.

1. In a first embodiment, the invention provides a plurality of polynucleotides, wherein each polynucleotide comprises:
a first target-specific domain and a second target-specific domain configured to bind to a first target sequence and a second target sequence, respectively, of a nucleic acid target, and
a unique molecule identifier (UMI) and a linker between the first and second target-specific domains,
wherein the first and second target-specific domains are configured to be connected to each other such that the polynucleotide forms a circle, optionally after a polymerase-mediated extension of the first or second target-specific domain, and
wherein the nucleic acid target comprises a polymorphic nucleotide within the first target sequence and/or the second target sequence, or between the first and second target sequences.

2. The plurality of polynucleotides of embodiment 1, which are single-stranded polynucleotides.

3. The plurality of polynucleotides of embodiment 1 or 2, which comprise a nucleic acid, an oligonucleotide, a DNA molecule, a DNA with pseudo-complementary bases, a DNA or RNA with one or more protected bases, an RNA molecule, a BNA molecule, an XNA molecule, a LNA molecule, a PNA molecule, or a γPNA molecule, or a combination thereof.

4. The plurality of polynucleotides of any one of embodiments 1-3, which are between about 50 nucleotides and about 200 nucleotides in length, e.g., between about 90 nucleotides and about 100 nucleotides in length.

5. The plurality of polynucleotides of any one of embodiments 1-4, wherein the first target-specific domain and/or the second target-specific domain are between about 15 nucleotides and about 30 nucleotides in length, e.g., about 20 nucleotides in length.

6. The plurality of polynucleotides of any one of embodiments 1-5, wherein the UMI is between about 5 and about 15 nucleotides in length, e.g., between about 6 and about 8 nucleotides in length.

7. The plurality of polynucleotides of any one of embodiments 1-6, wherein the linker is between about 10 and about 100 nucleotides in length, e.g., about 50 nucleotides in length.

8. The plurality of polynucleotides of any one of embodiments 1-7, wherein the linker comprises one or more common nucleotides for subsequent PCR annealing. Any suitable nucleotide or short nucleotide sequence that will facilitate PCR can be used.

9. The plurality of polynucleotides of any one of embodiments 1-8, wherein the distance between the first and the second target sequences are between about 0 and about 100 nucleotides.

10. The plurality of polynucleotides of any one of embodiments 1-9, wherein each polynucleotide comprises the first target-specific domain, the UMI, the linker, and the second target-specific domain in the 5′ to 3′ direction.

11. The plurality of polynucleotides of any one of embodiments 1-9, wherein each polynucleotide comprises the first target-specific domain, the linker, the UMI, and the second target-specific domain in the 5′ to 3′ direction.

12. The plurality of polynucleotides of any one of embodiments 1-11, wherein the nucleic acid target is from a sex chromosome, such as a chromosome X or chromosome Y, or from an autosome.

13. The plurality of polynucleotides of any one of embodiments 1-12, wherein the nucleic acid target is from a mammalian chromosome, such as a human chromosome.

14. The plurality of polynucleotides of any one of embodiments 1-13, wherein the plurality of polynucleotides are configured to bind to a target sequence on human chromosome 1, human chromosome 2, human chromosome 3, human chromosome 4, human chromosome 9, human chromosome 13, human chromosome 15, human chromosome 18, human chromosome 19, human chromosome 21, human chromosome 22, human chromosome X, or human chromosome Y, or any combination thereof.

15. The plurality of polynucleotides of embodiments14, wherein the plurality of polynucleotides are configured to bind to a target sequence on human chromosome 21, human chromosome 18, human chromosome 13, human chromosome X, human chromosome Y, or at least one other human autosome, e.g., human chromosome 1, human chromosome 2, human chromosome 3, human chromosome 4, human chromosome 9, human chromosome 15, human chromosome 19, human chromosome 21, or any combination thereof.

16. The plurality of polynucleotides of embodiment 15, wherein the plurality of polynucleotides are configured to bind to a target sequence on human chromosome 21, human chromosome 18, human chromosome 13, human chromosome X, human chromosome Y, and at least one other human autosome.

17. The plurality of polynucleotides of any one of embodiments 1-16, wherein the polymorphic nucleotide is at a single nucleotide polymorphism (SNP) site.

18. The plurality of polynucleotides of any one of embodiments 1-17, wherein the polymorphic nucleotide comprises a plurality of polymorphic nucleotides, for example, nucleotides at a plurality of single nucleotide polymorphism (SNP) sites.

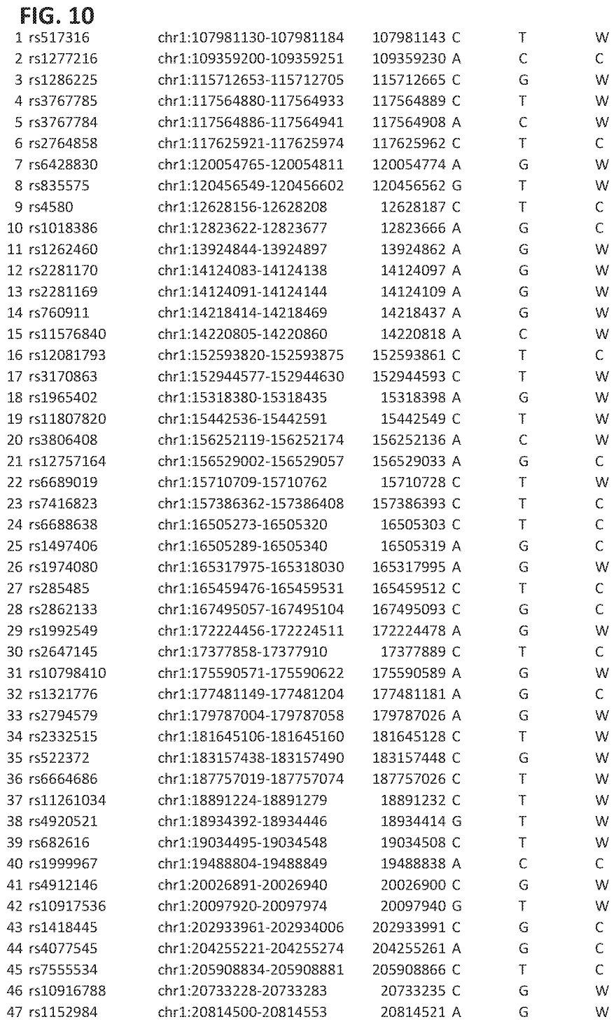

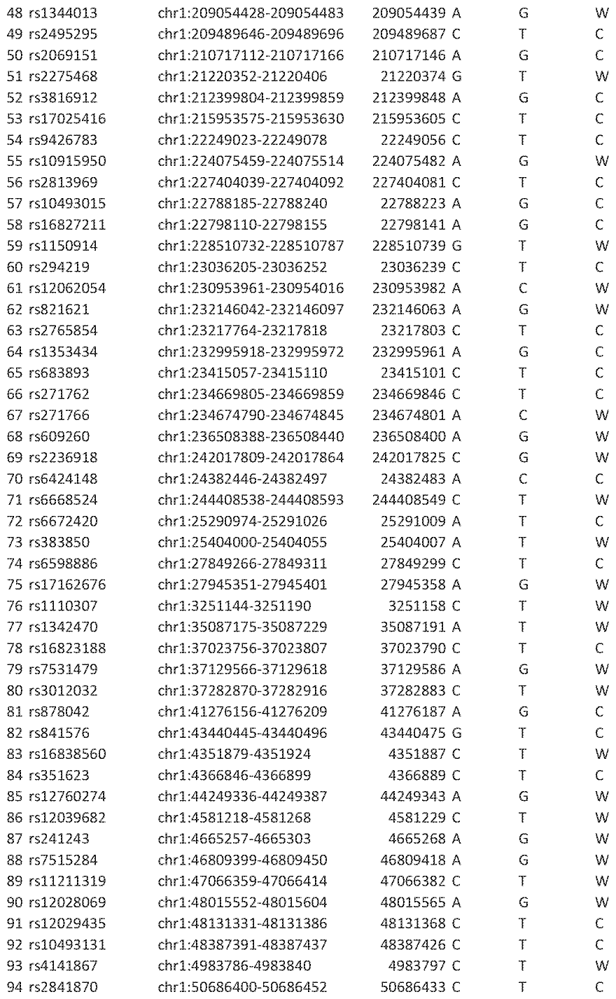

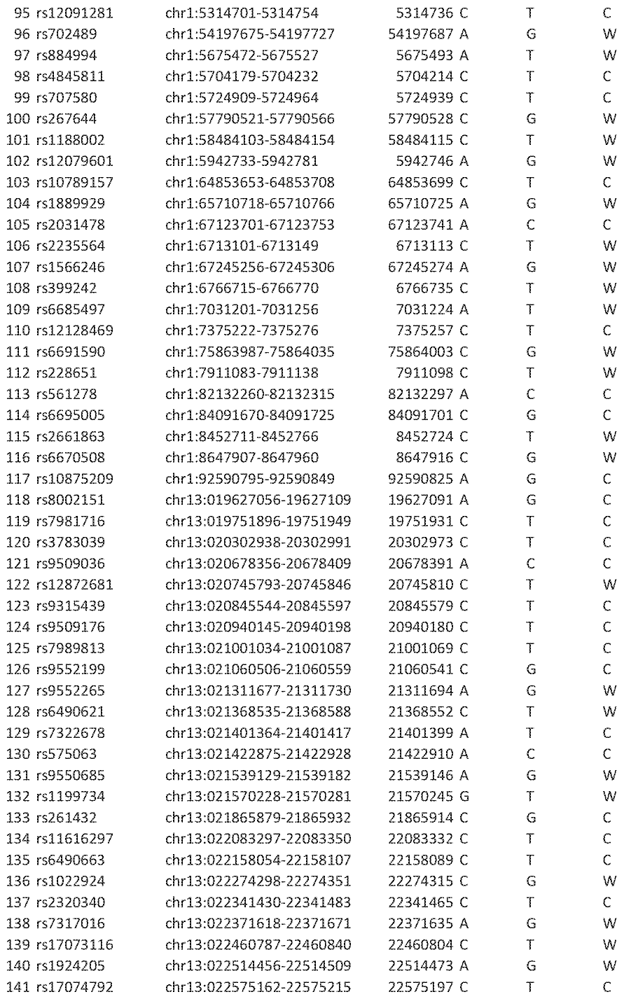

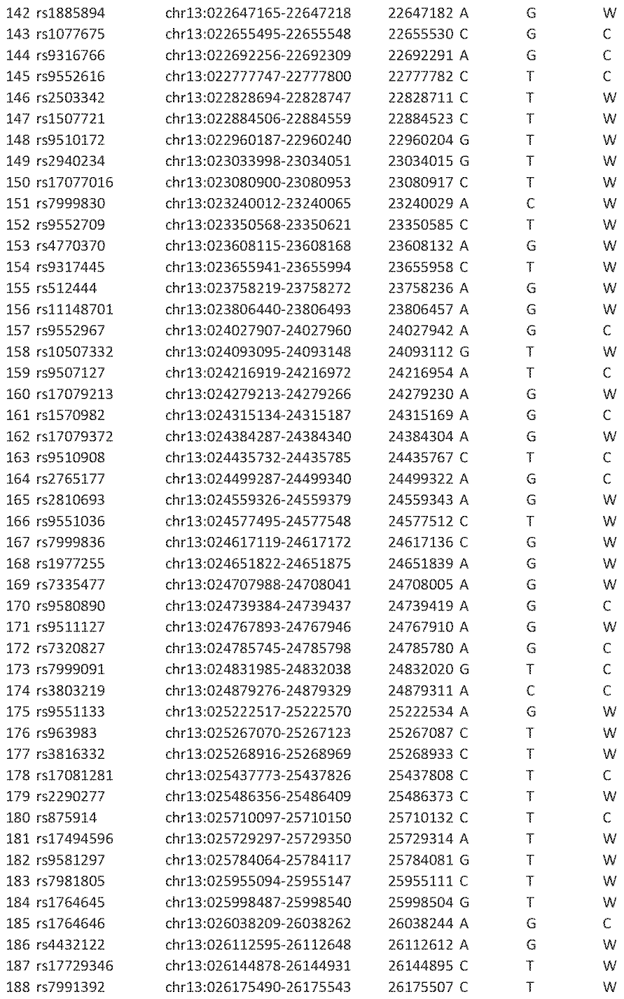

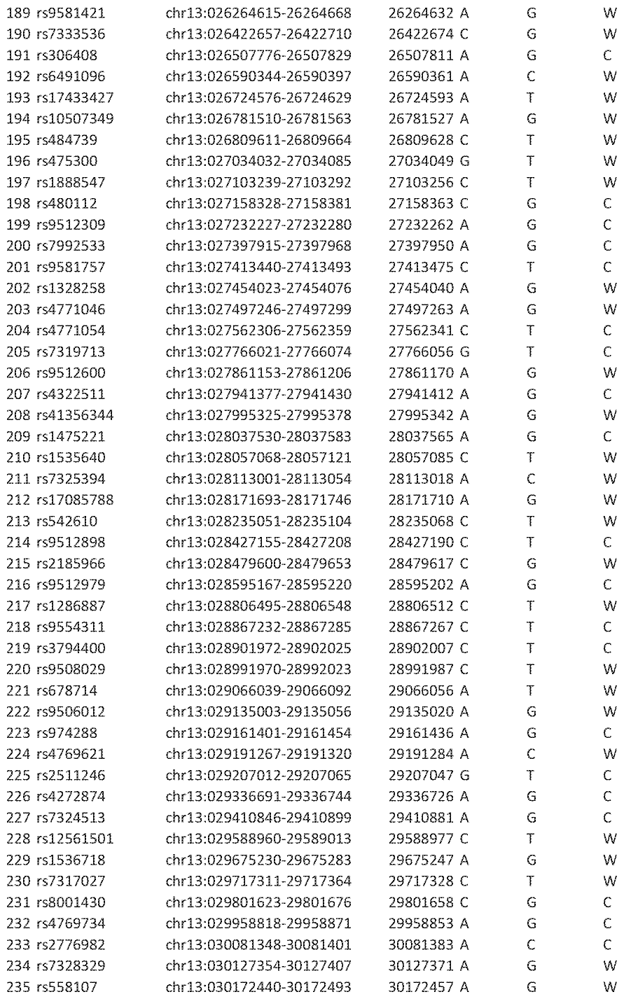

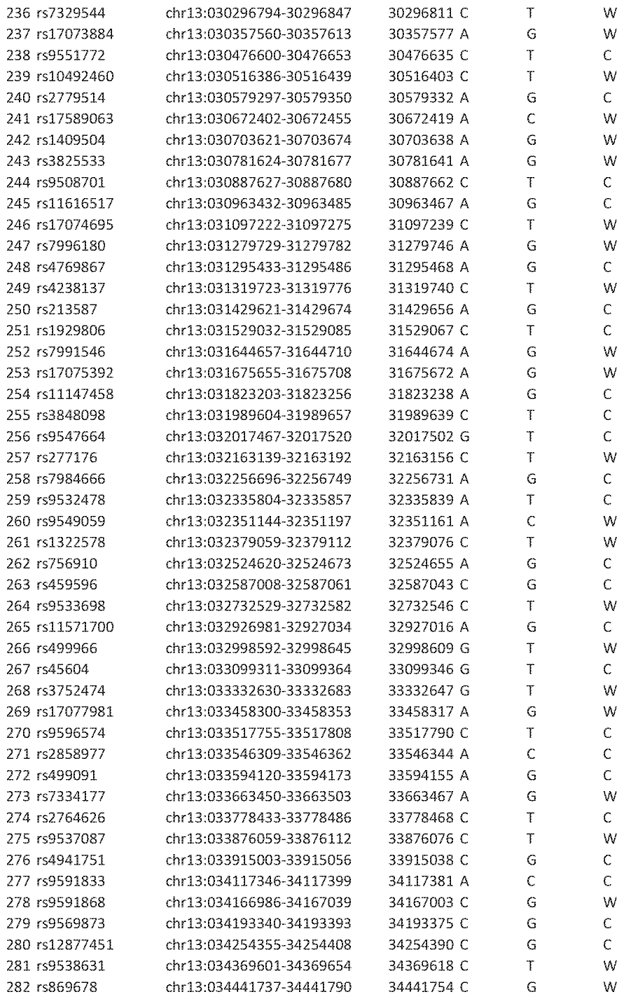

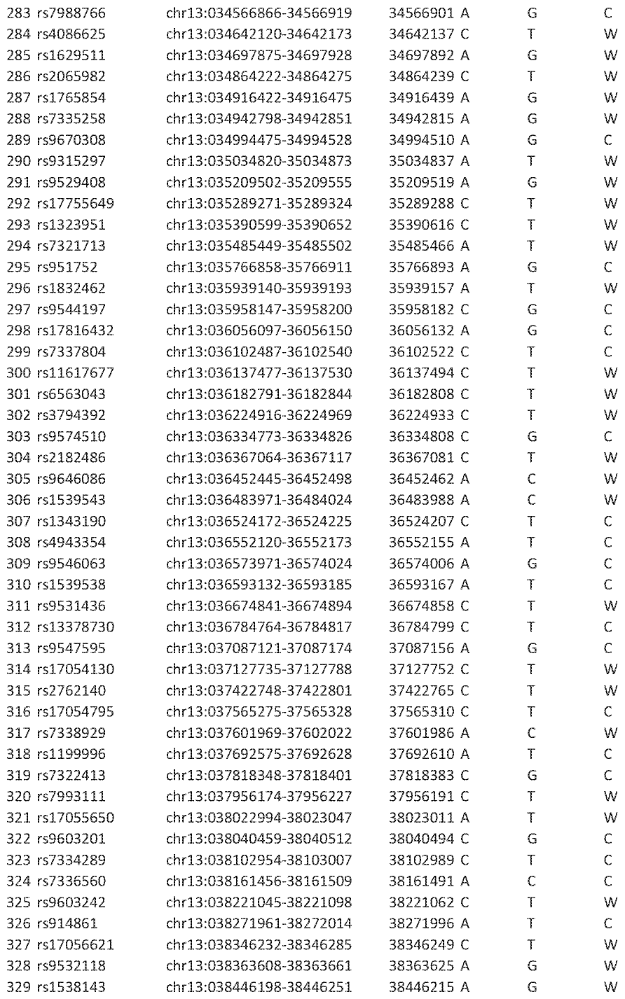

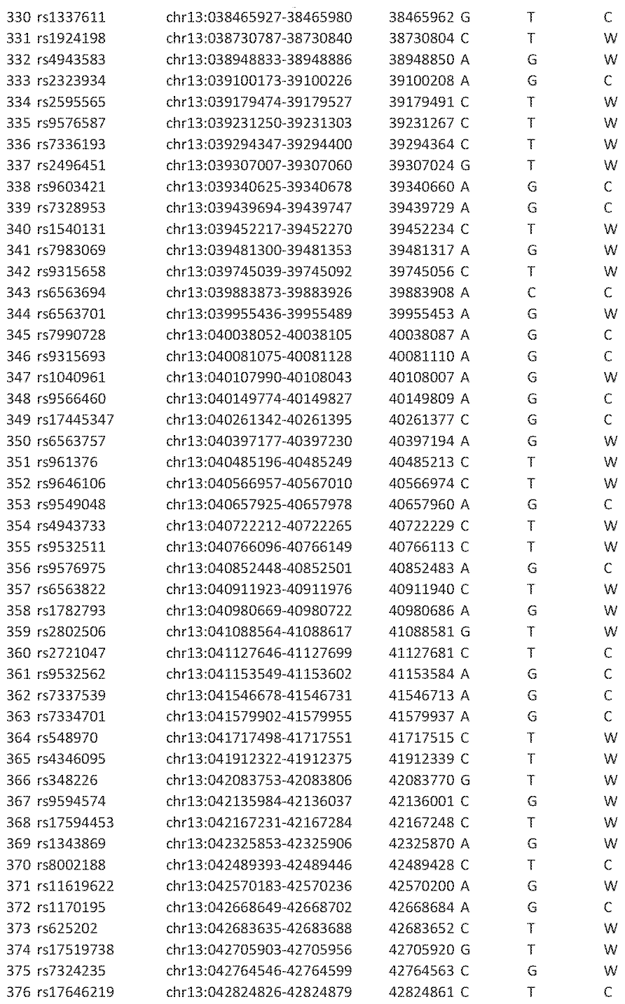

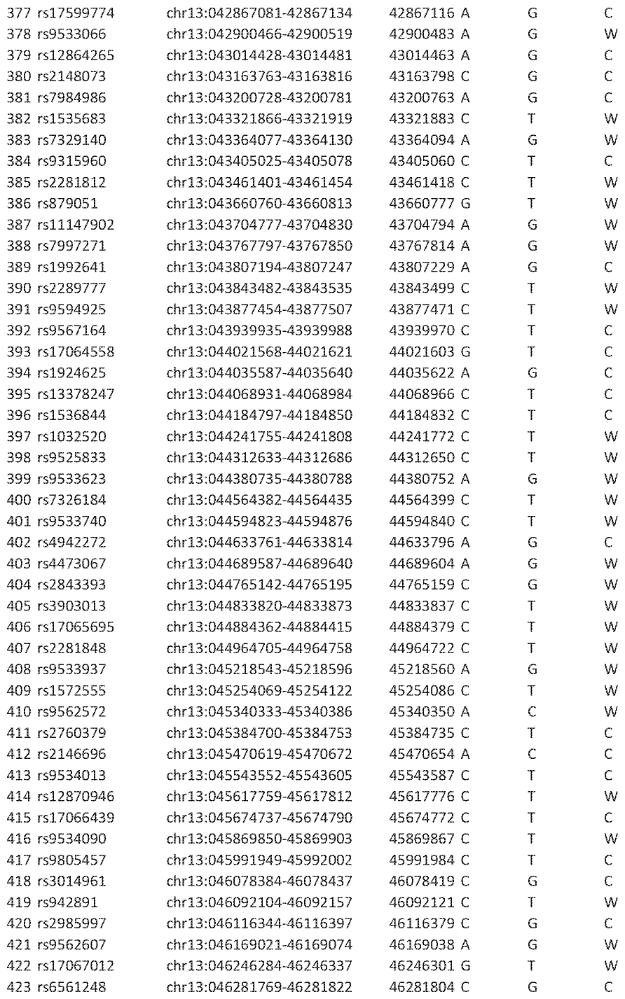

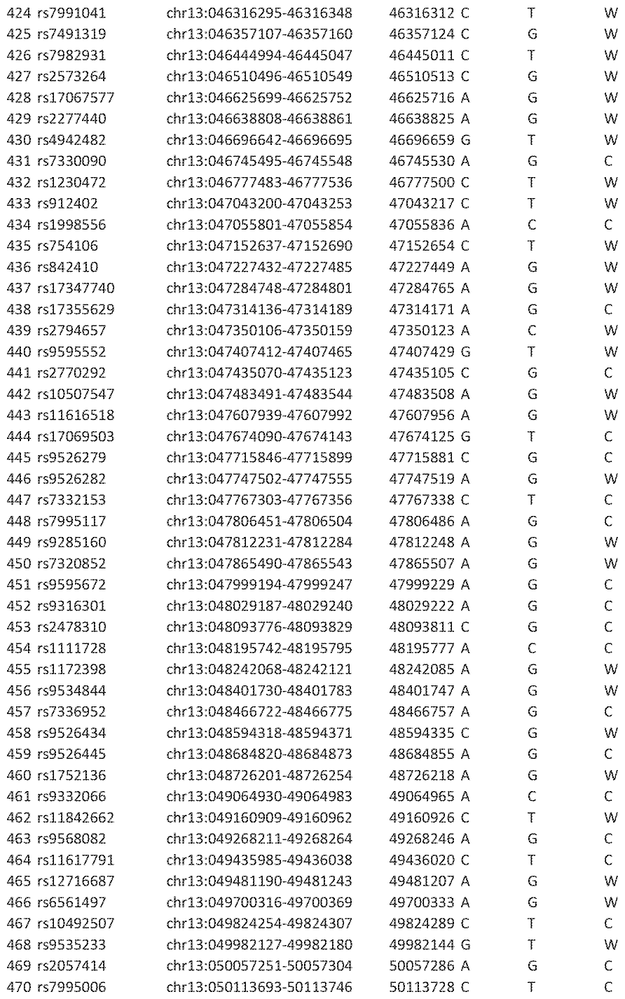

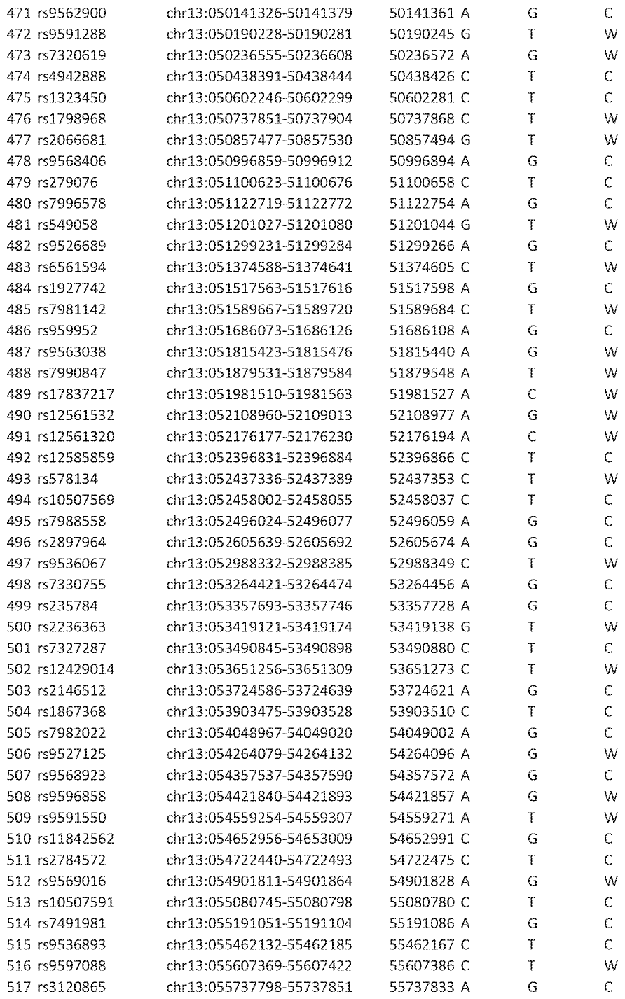

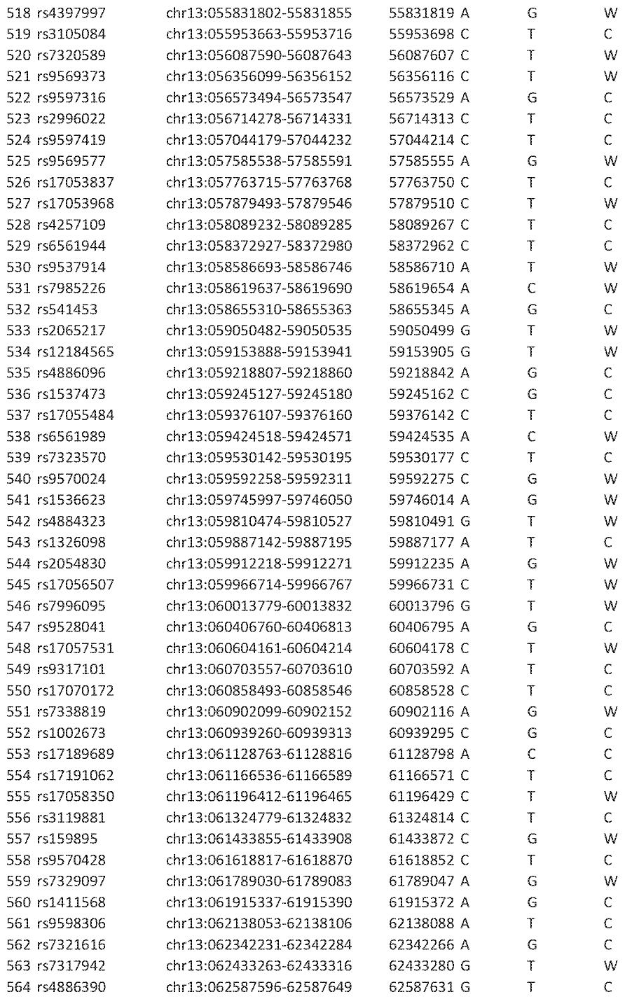

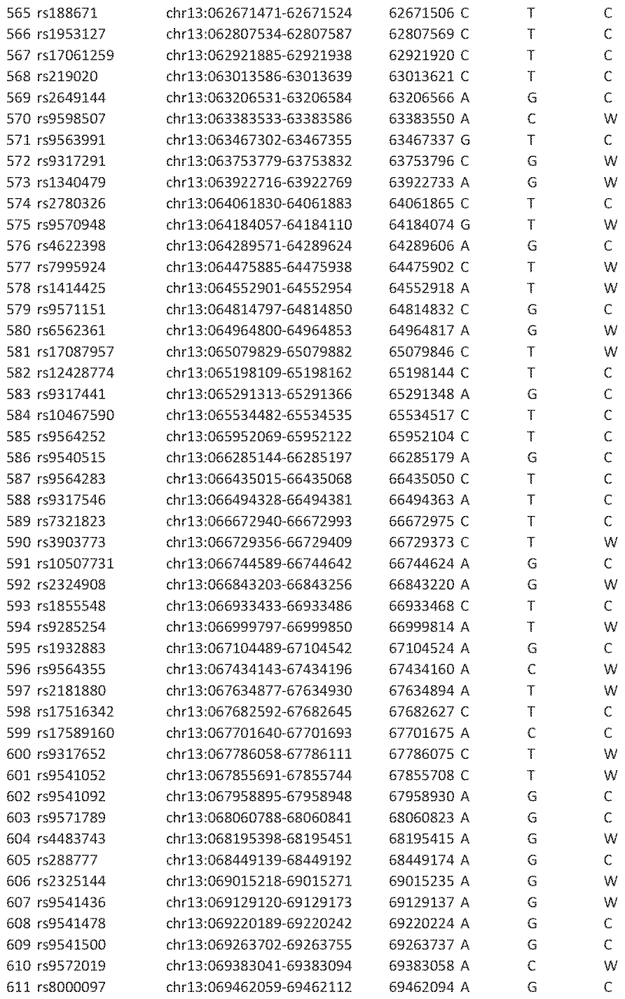

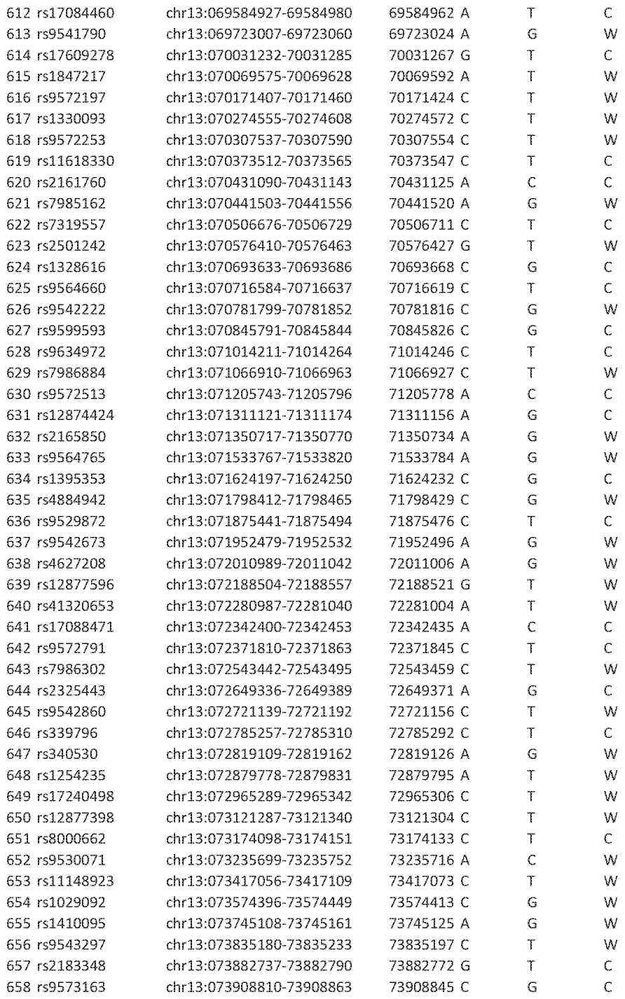

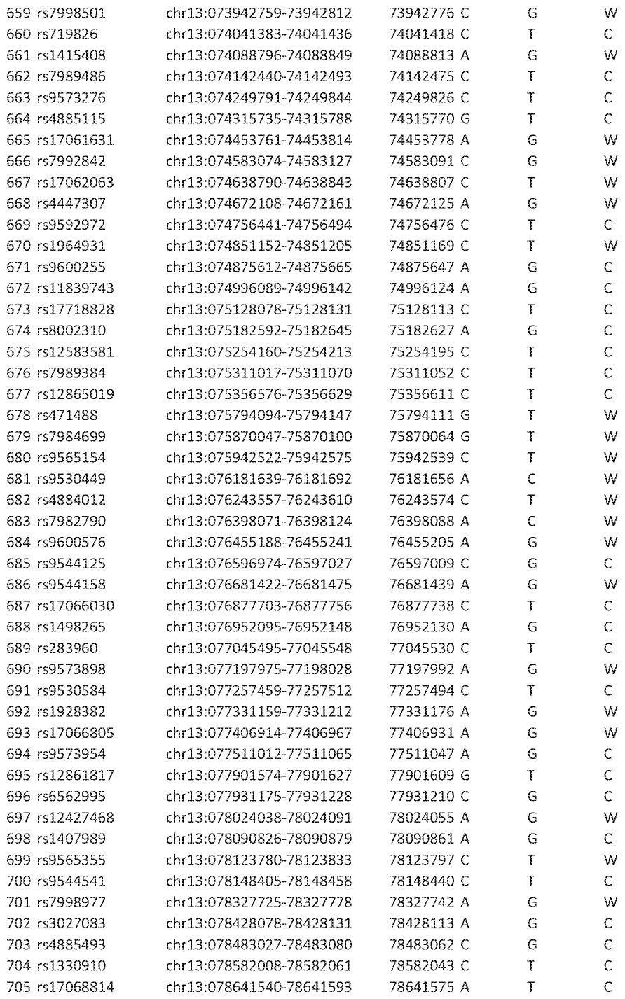

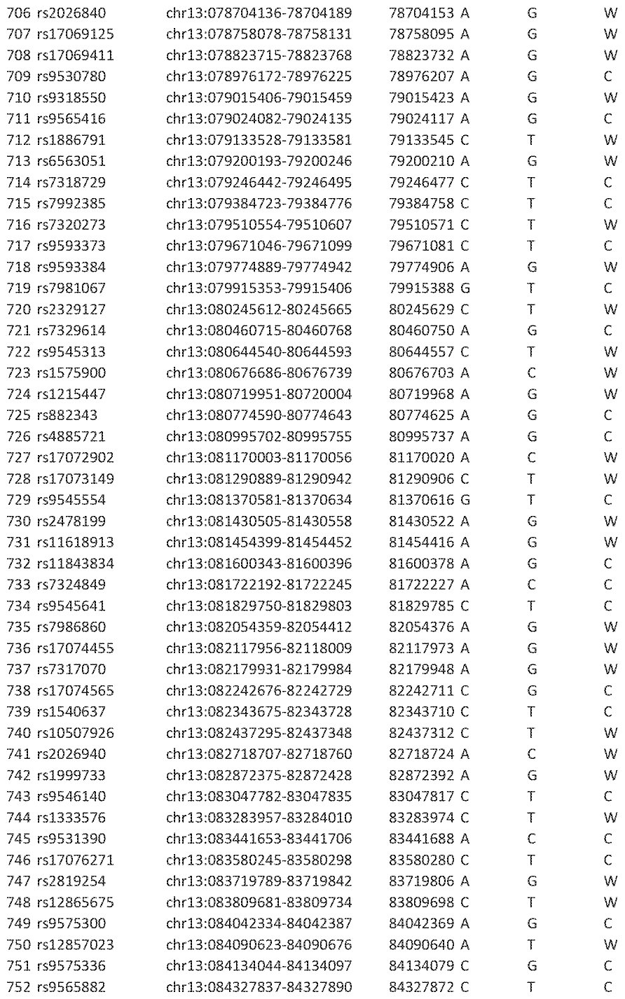

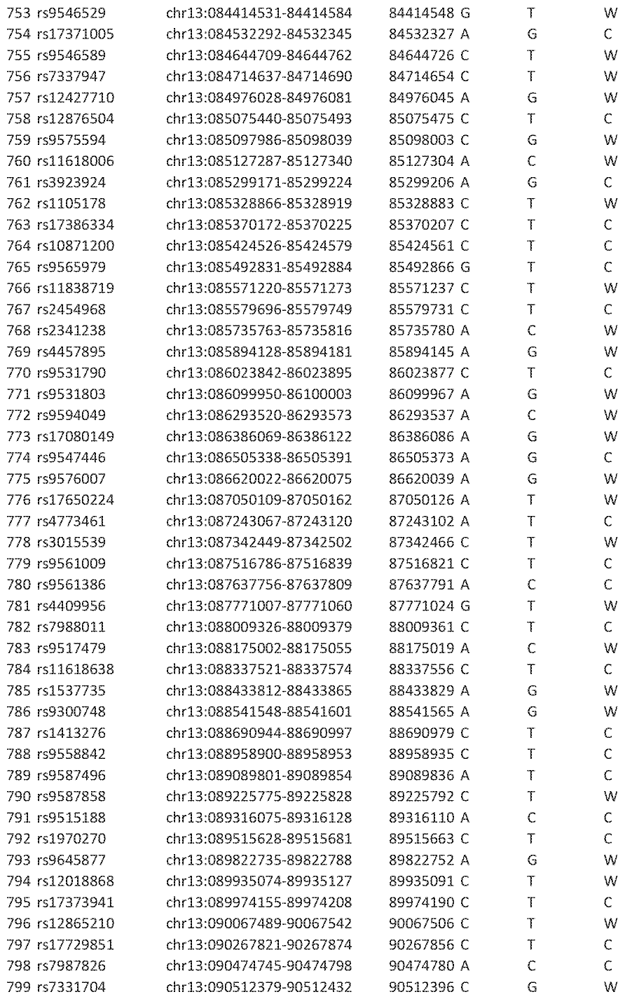

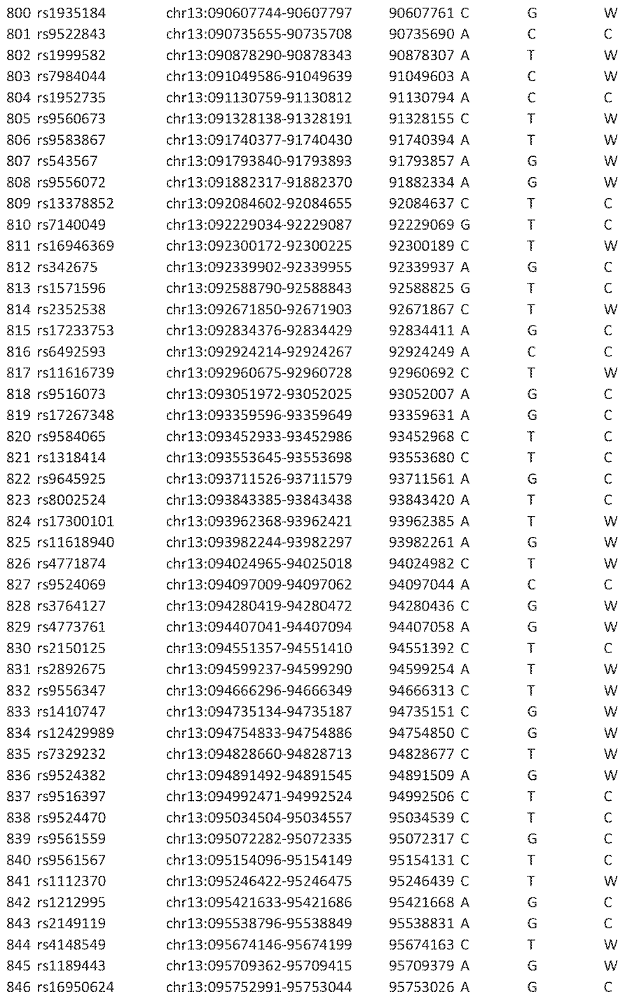

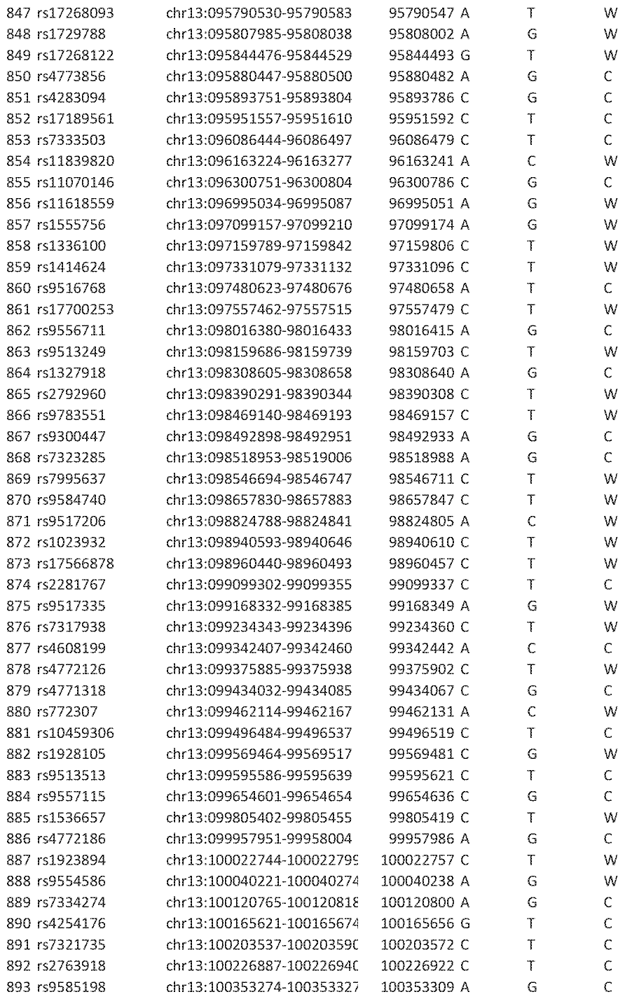

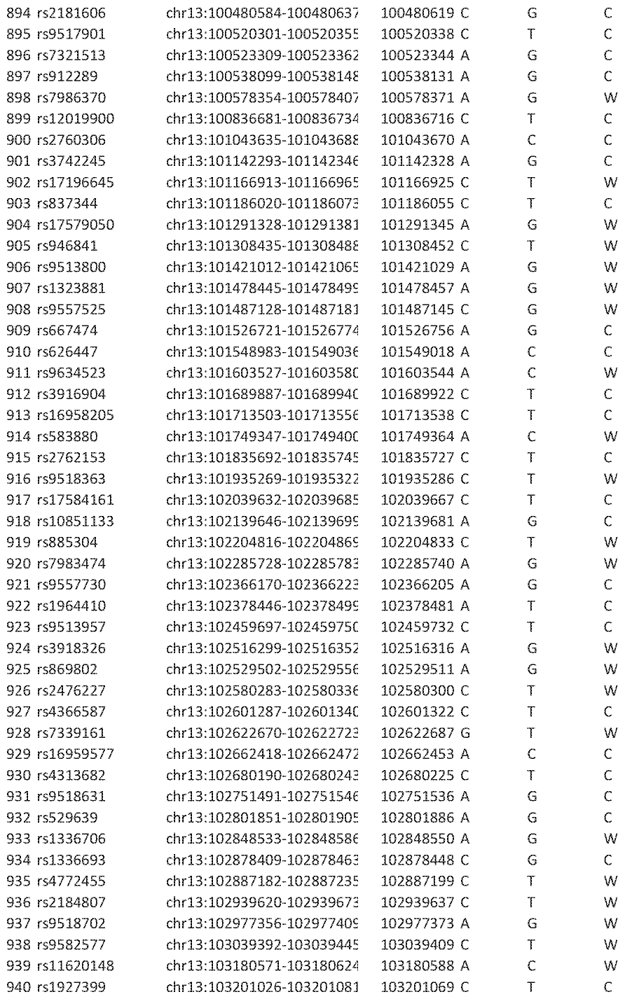

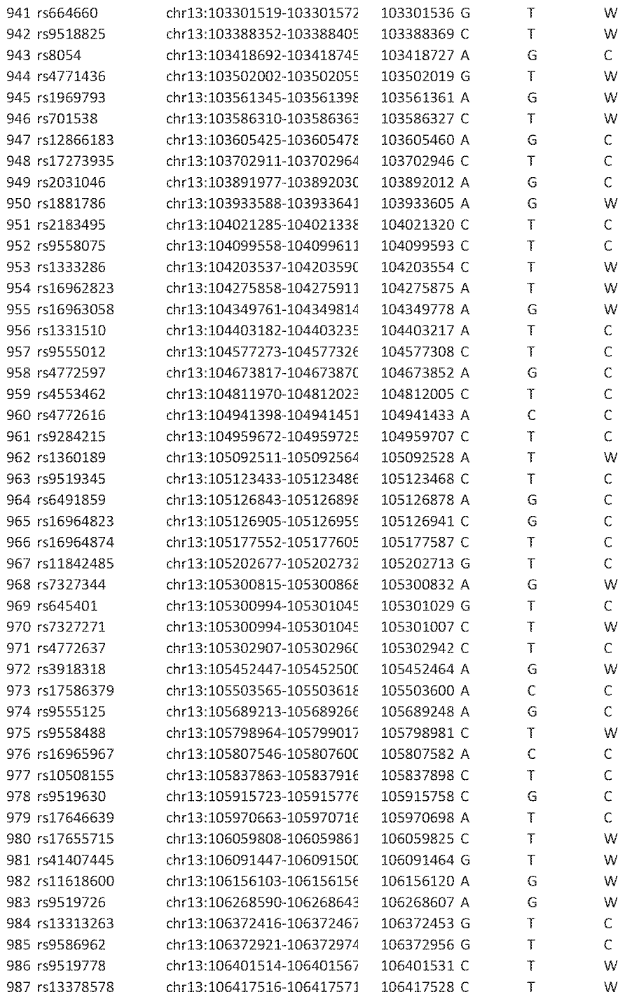

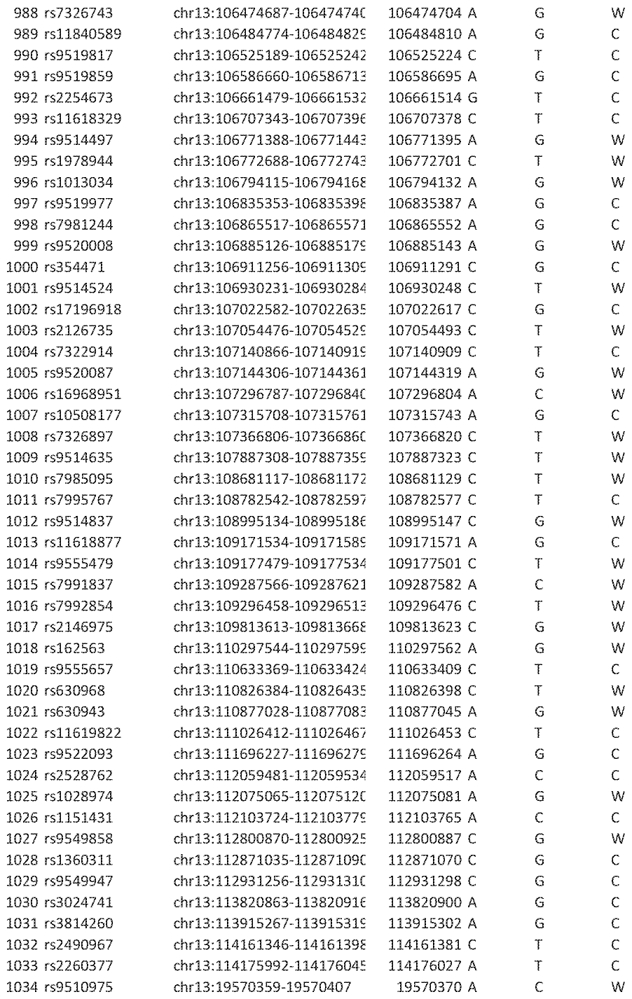

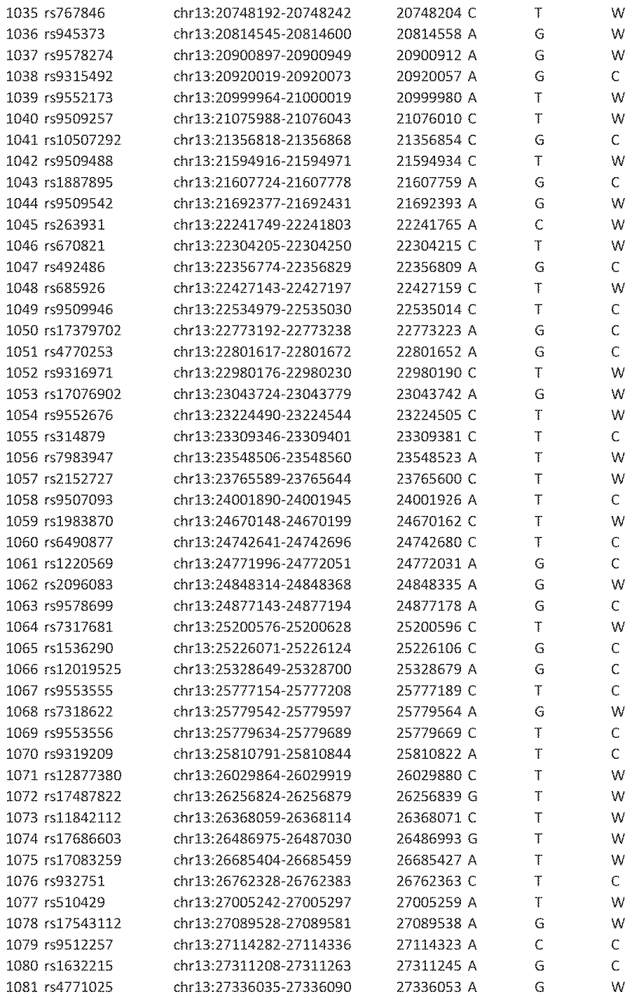

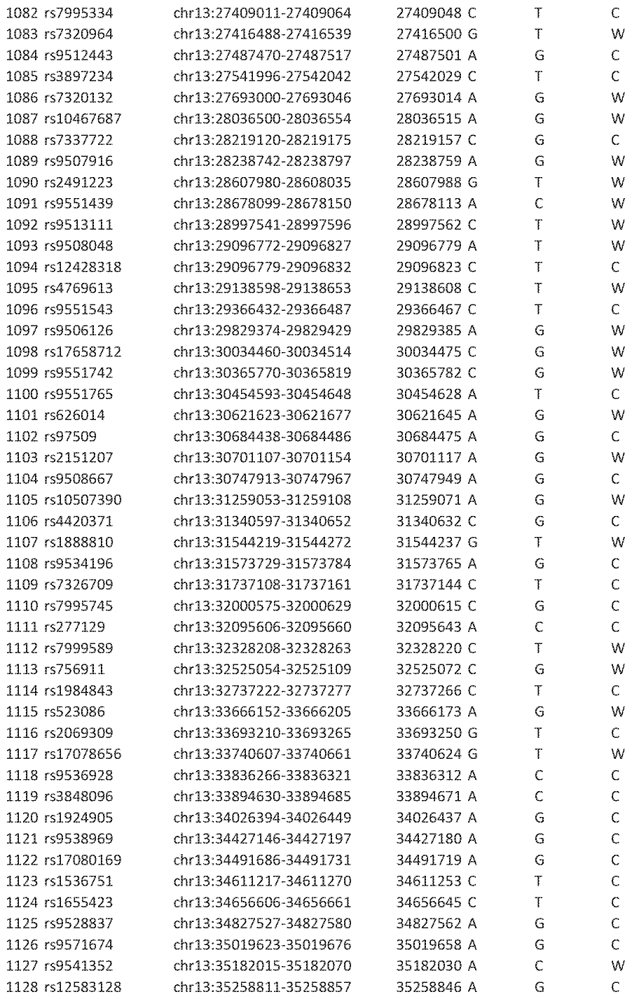

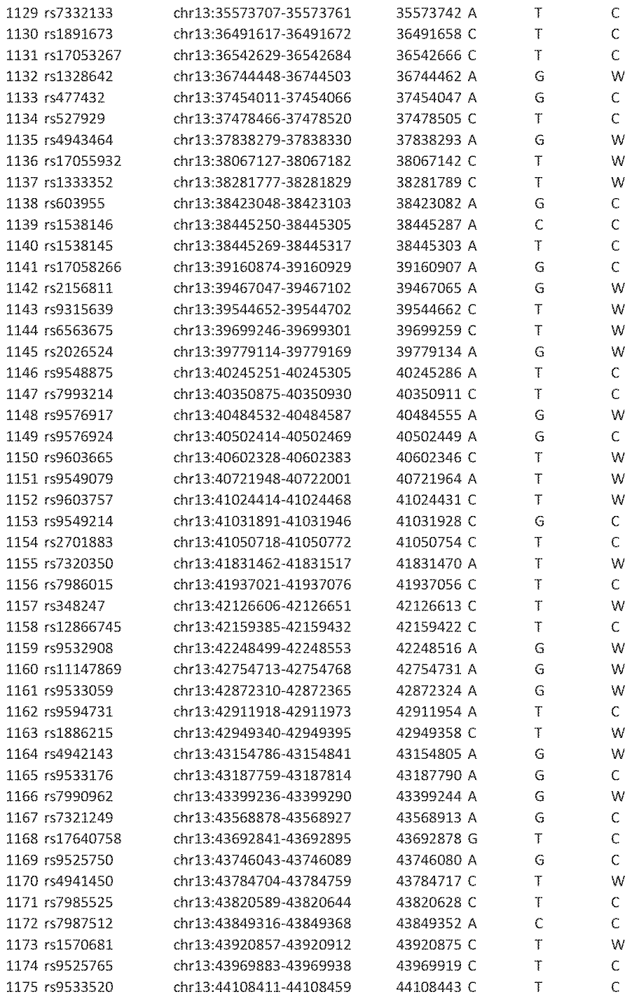

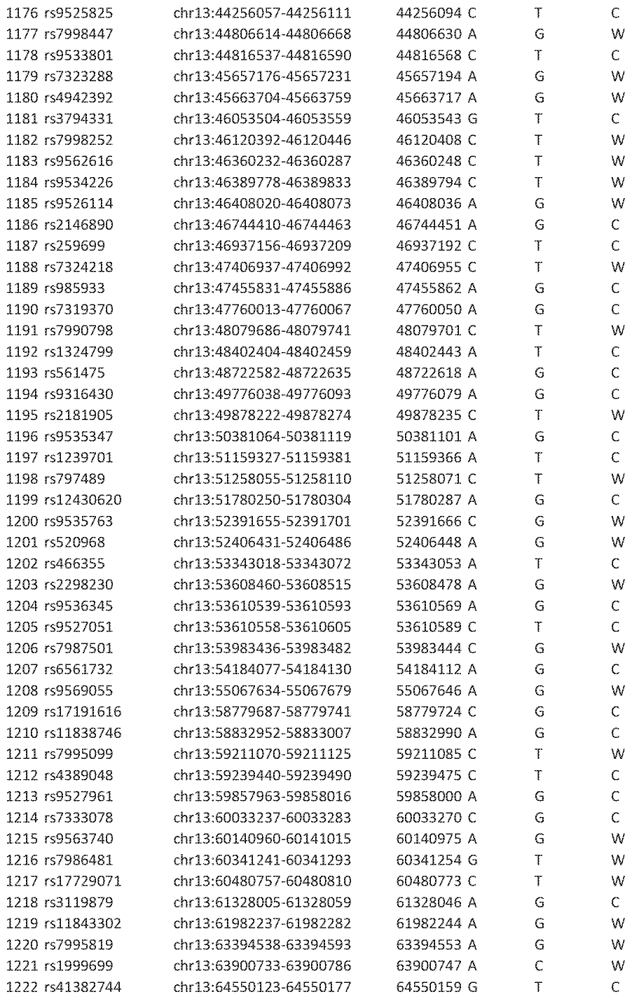

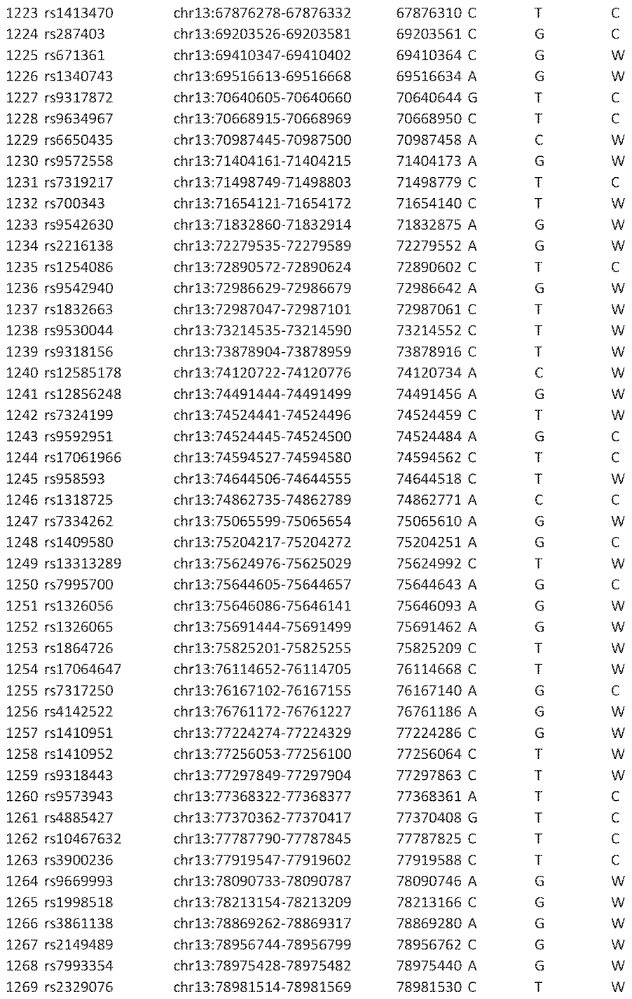

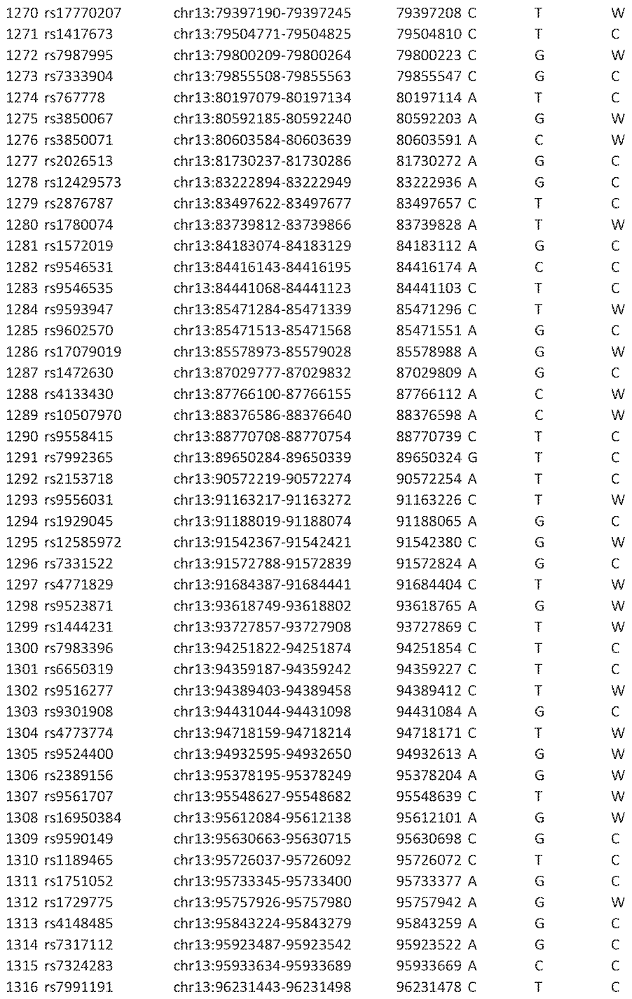

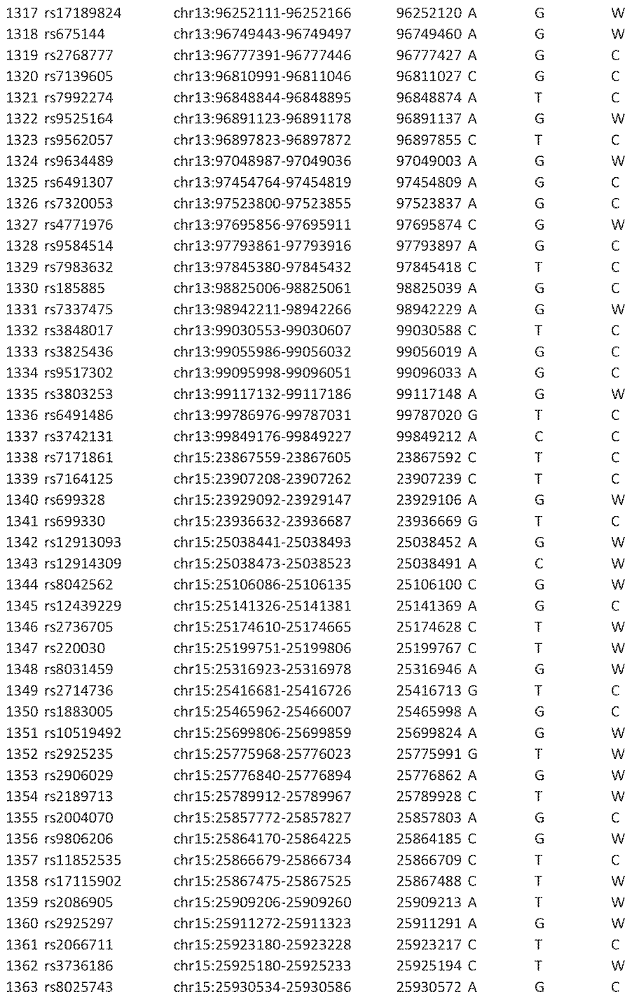

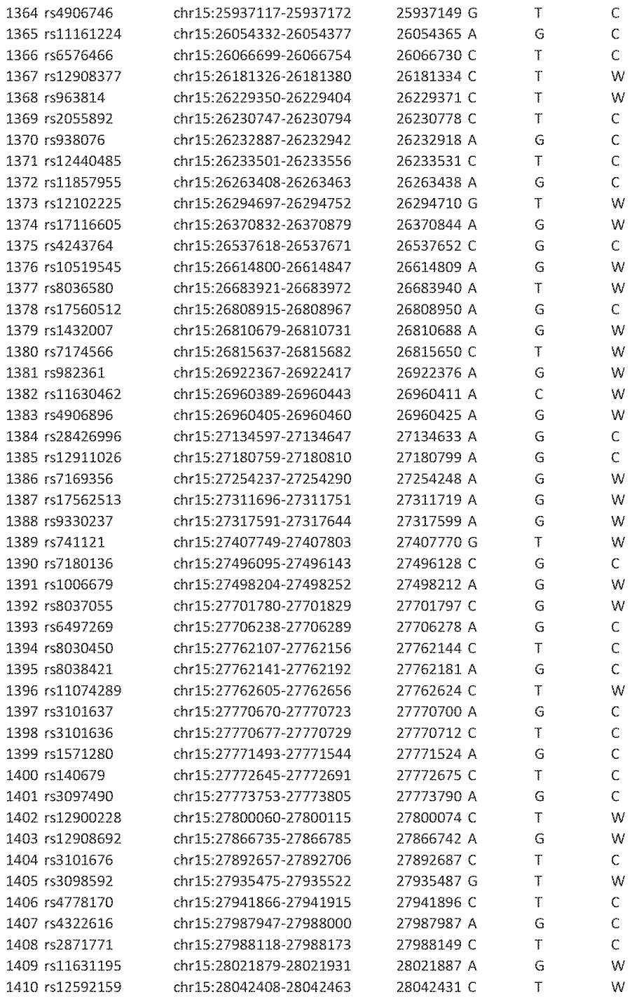

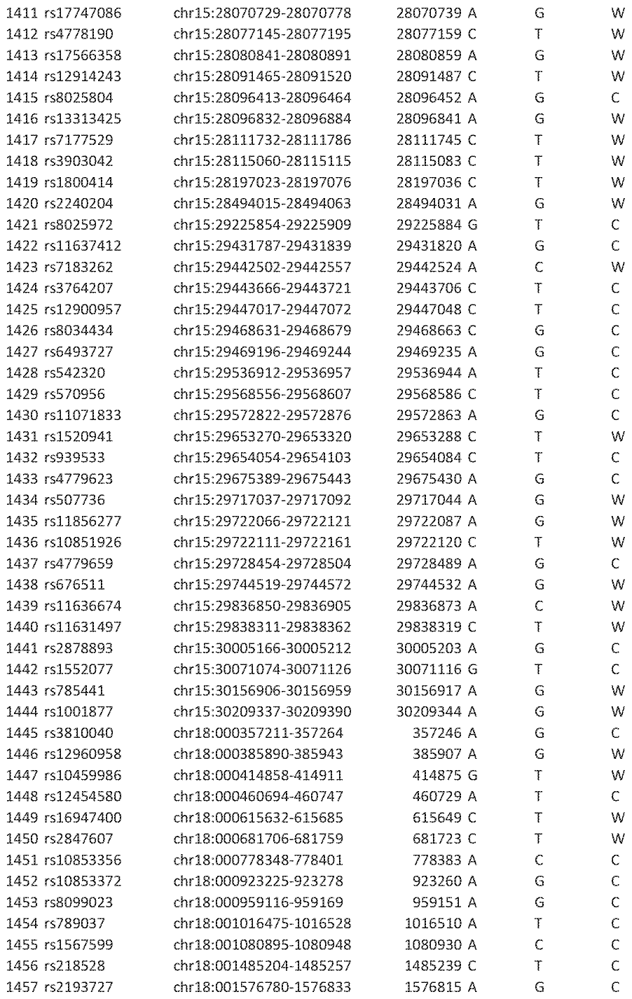

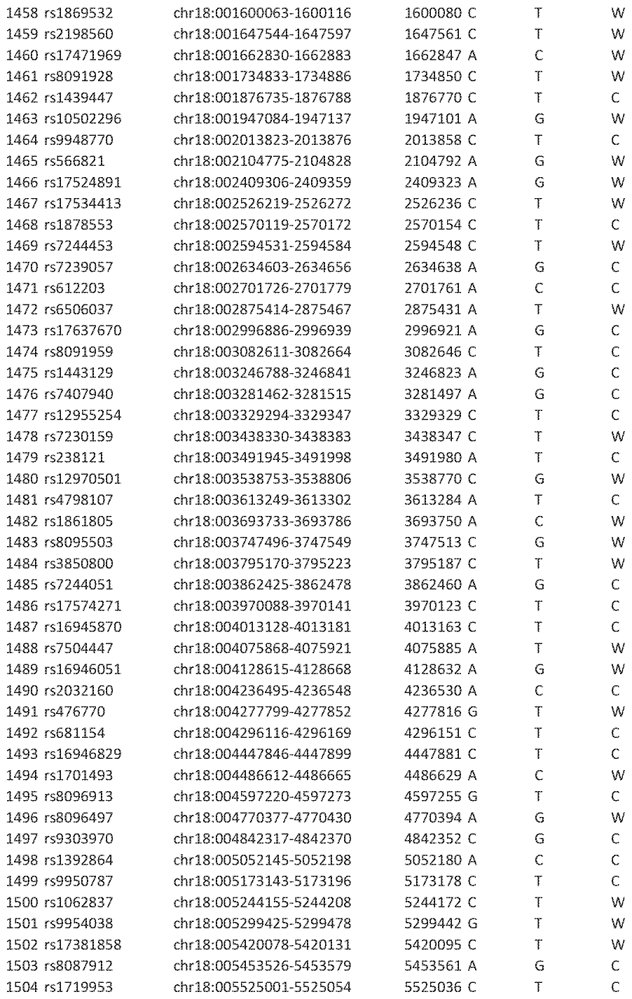

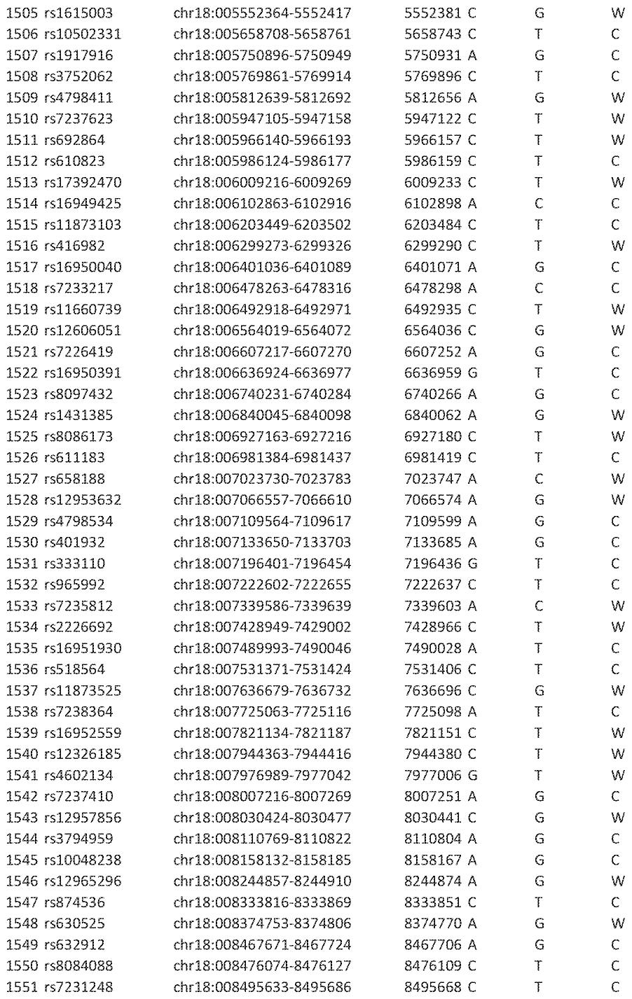

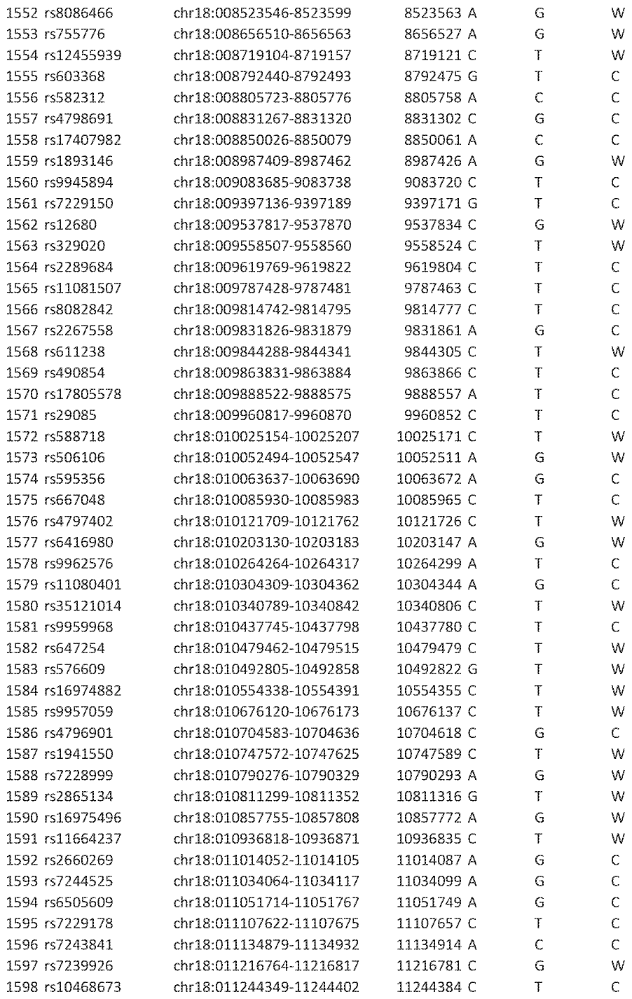

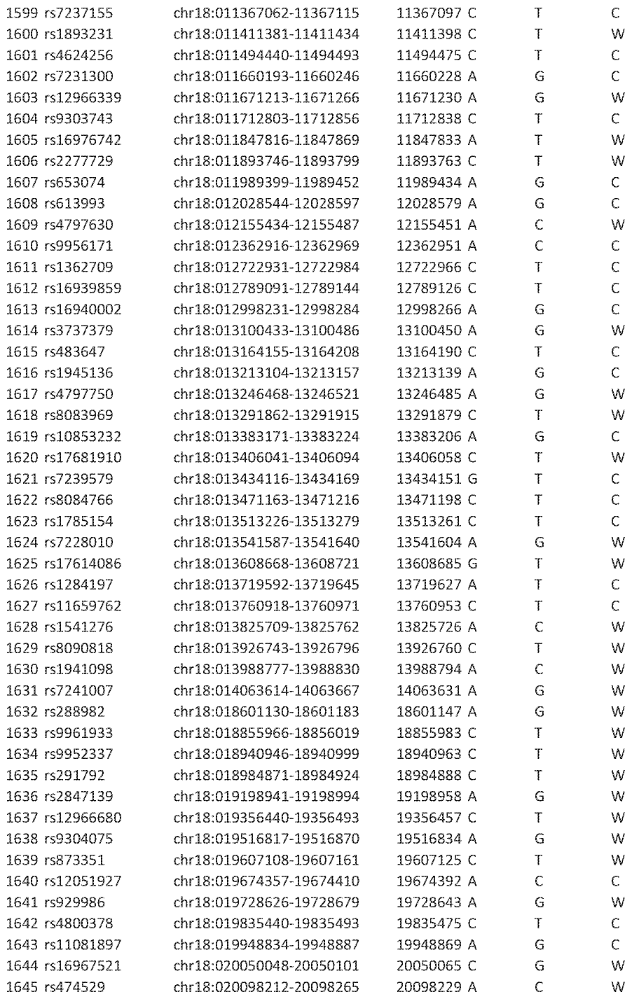

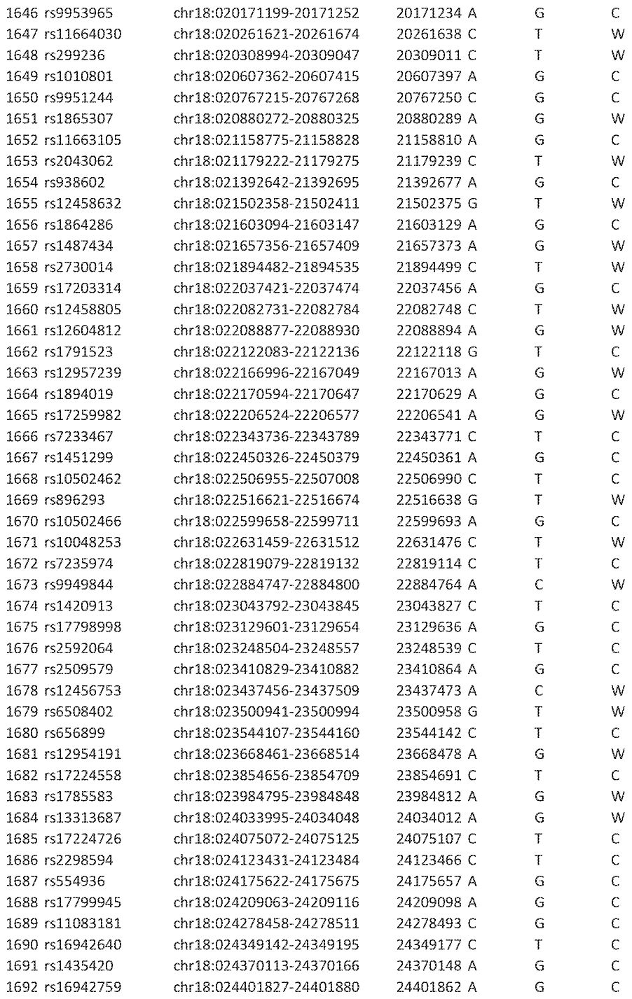

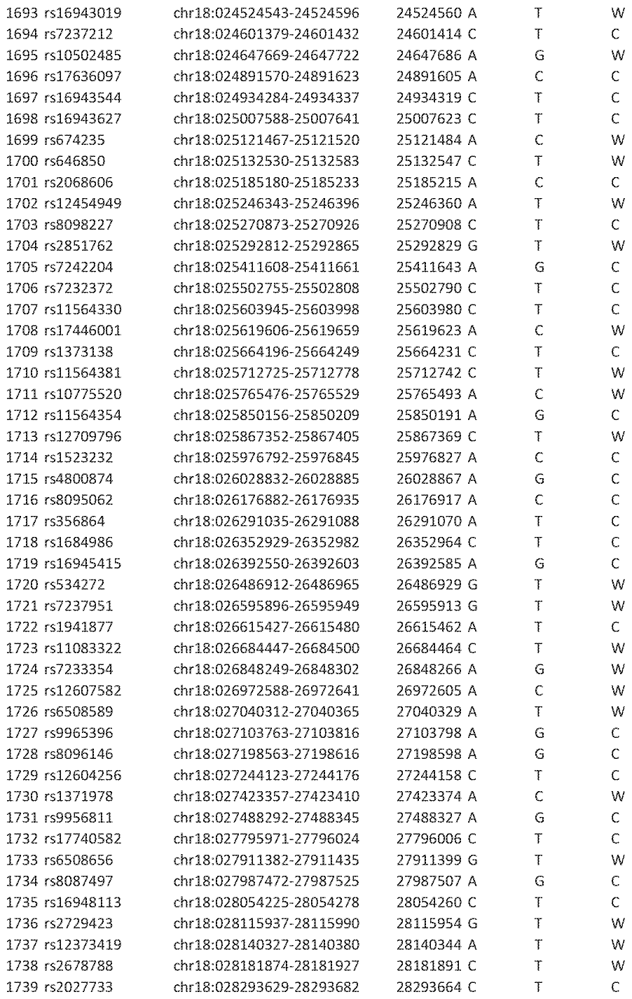

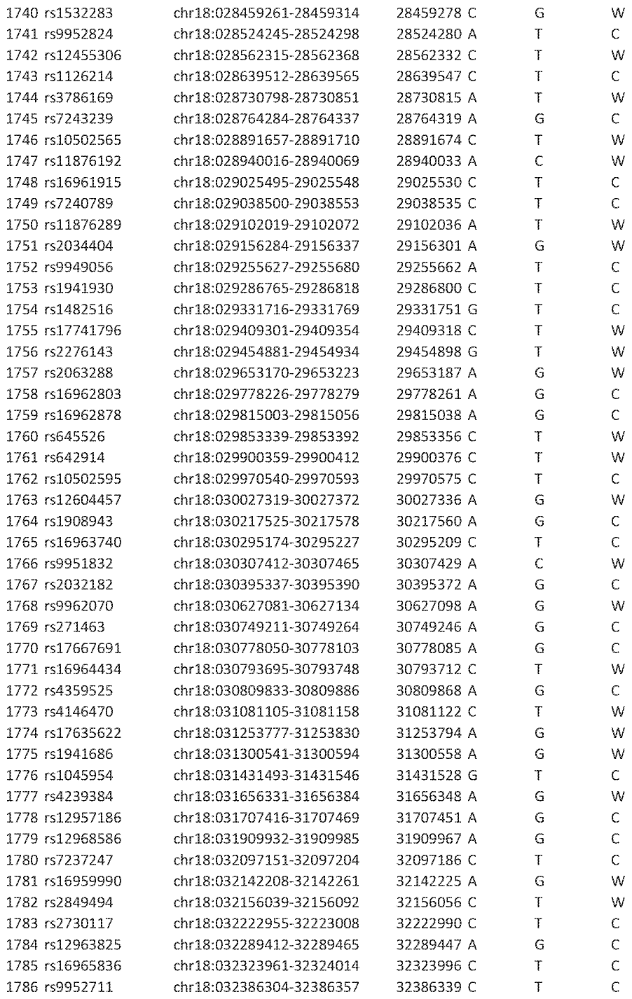

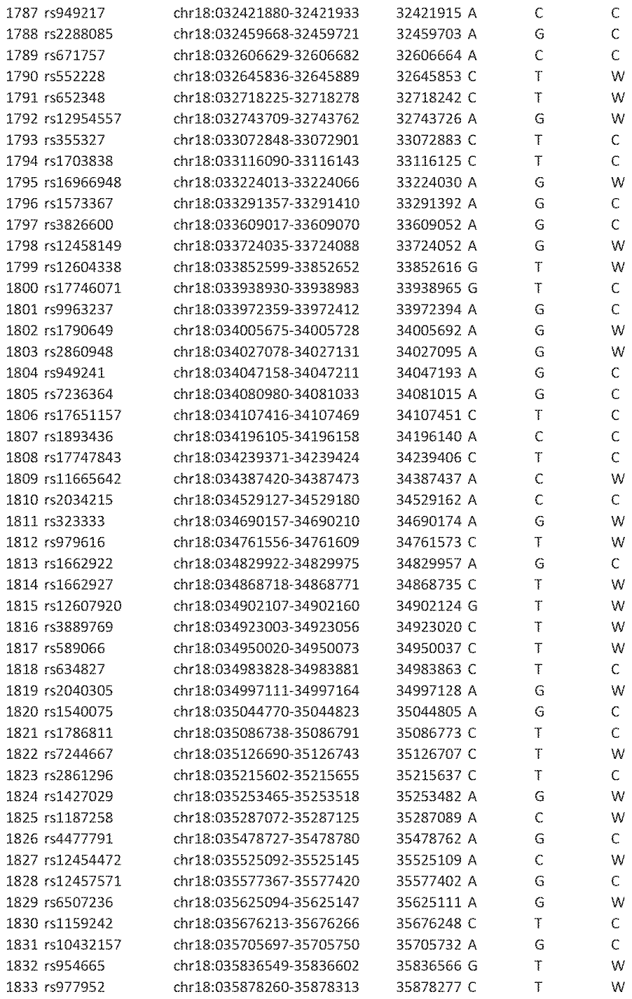

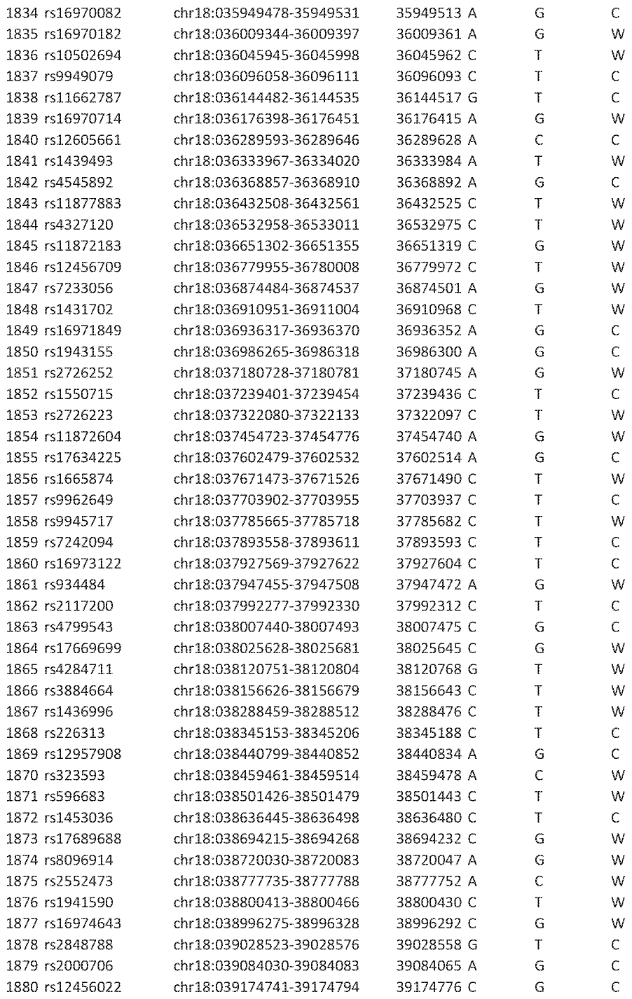

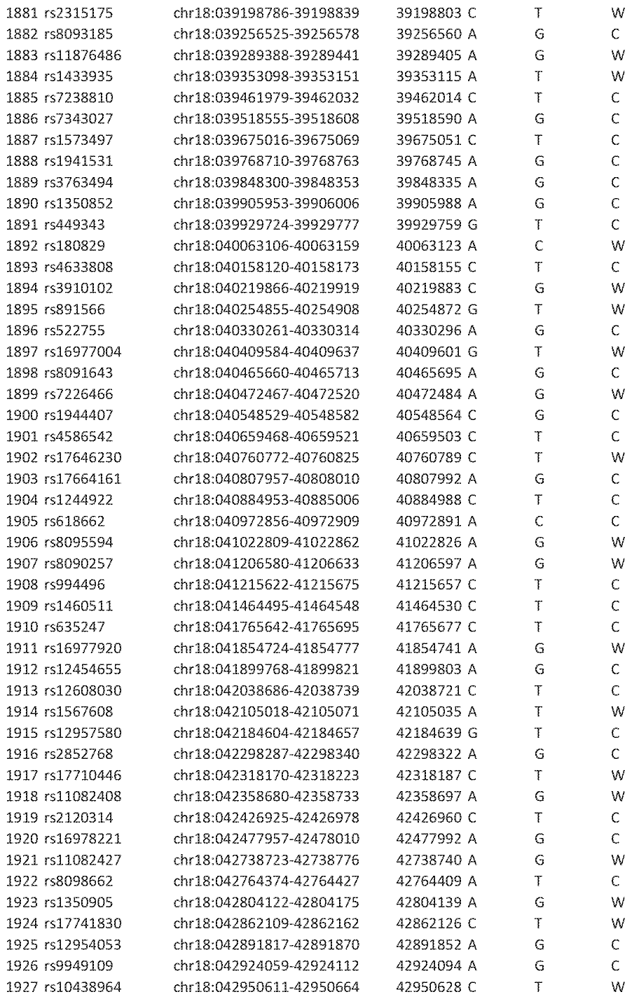

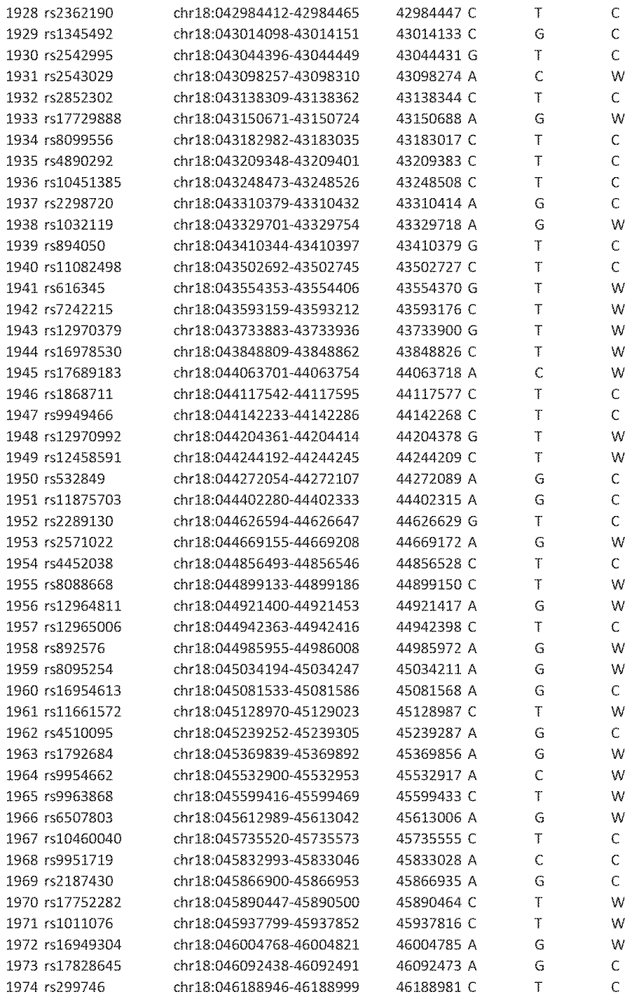

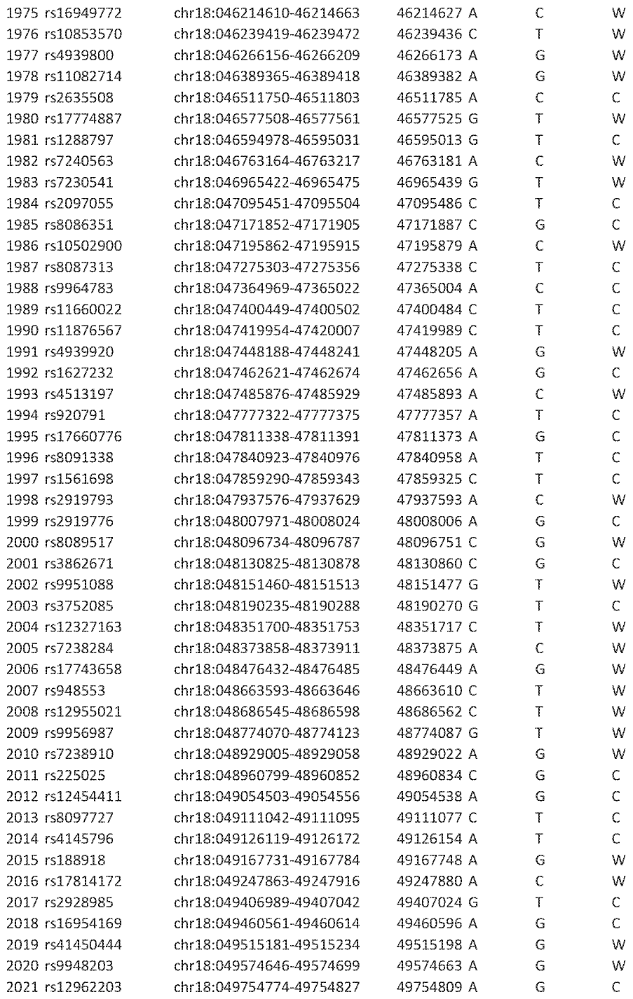

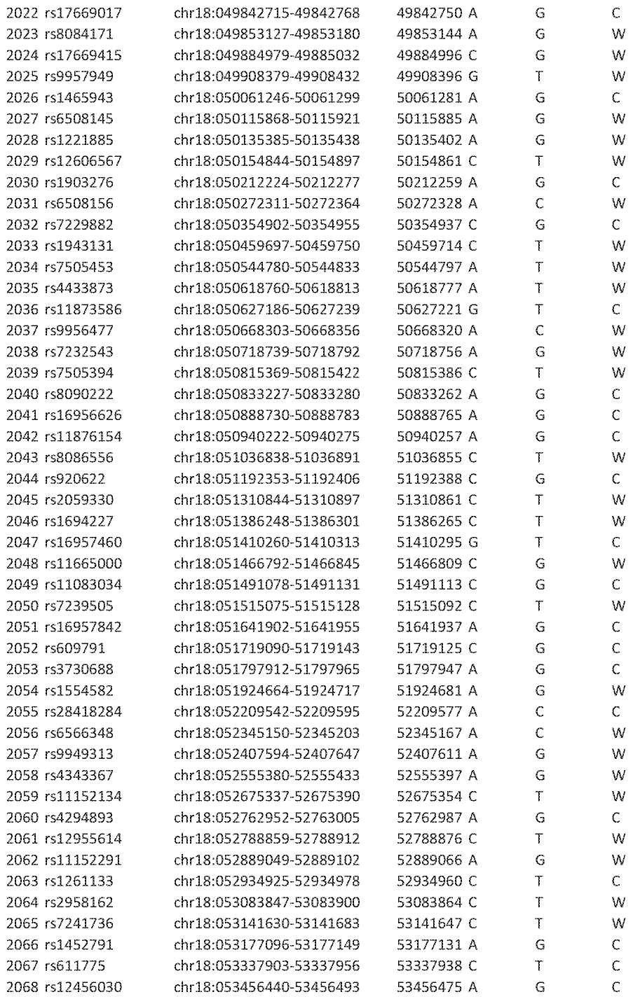

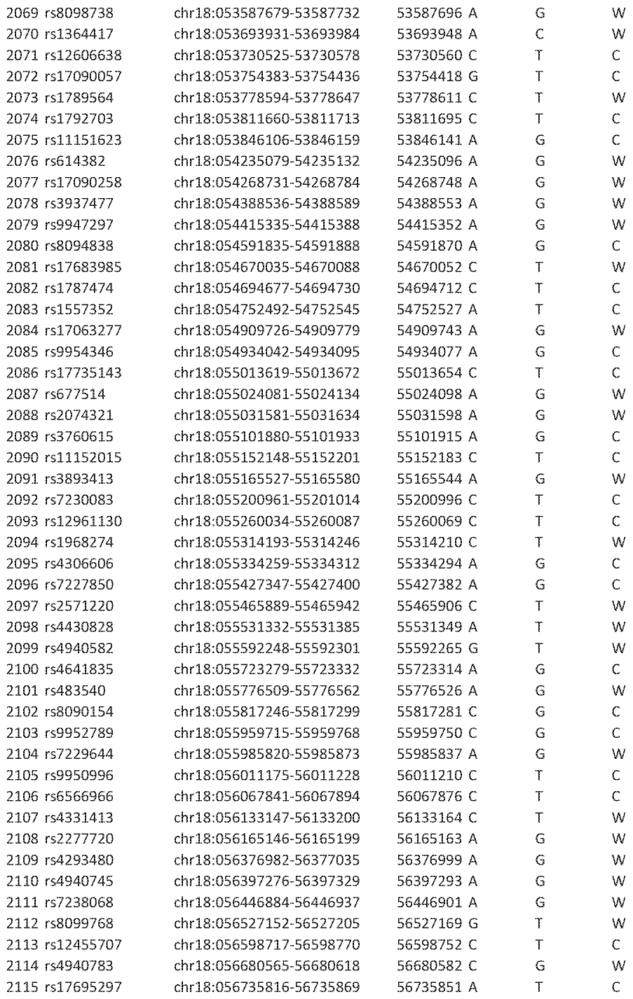

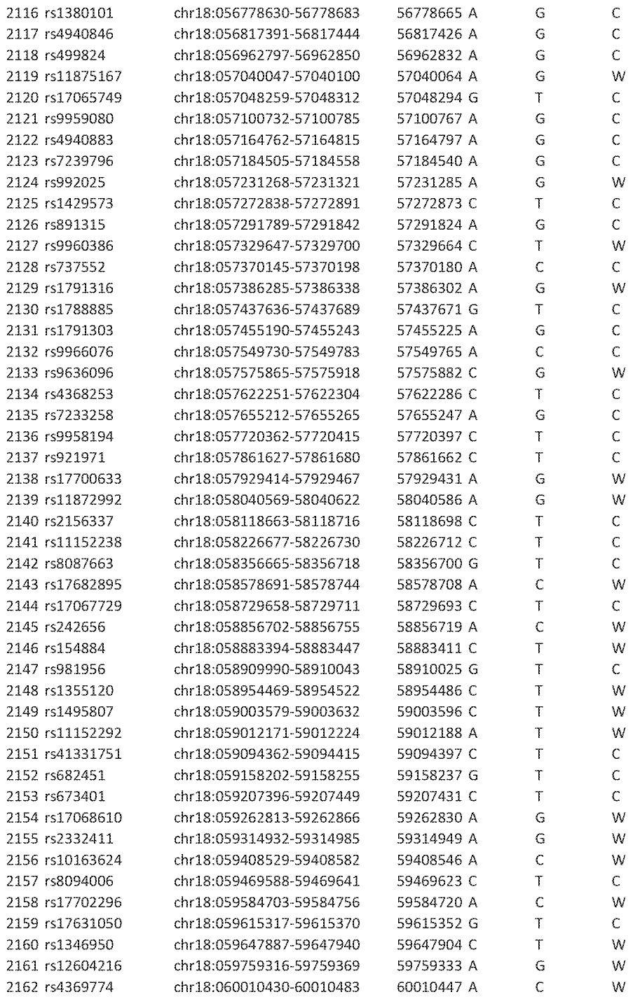

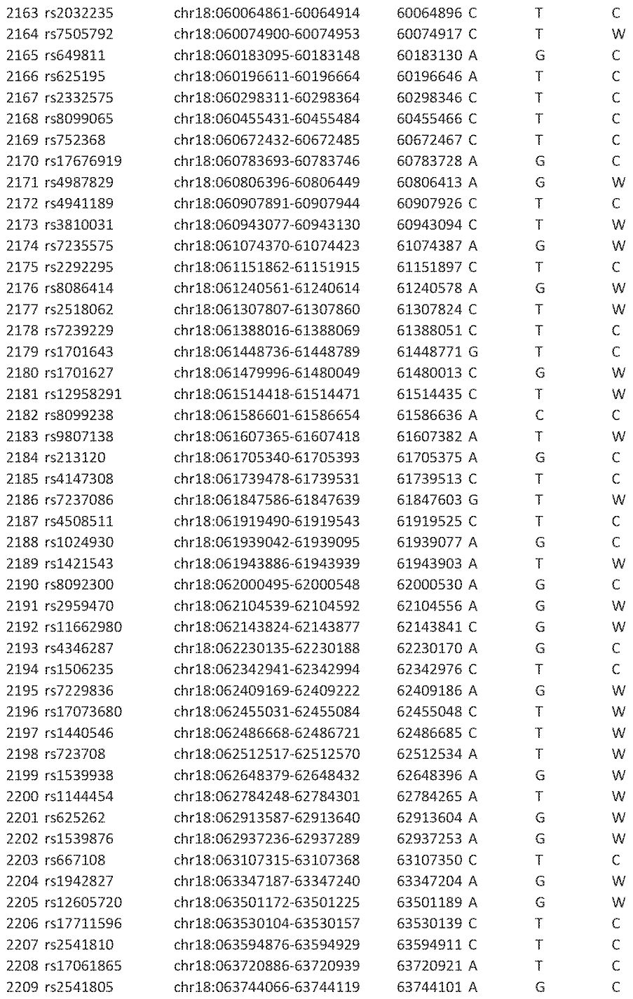

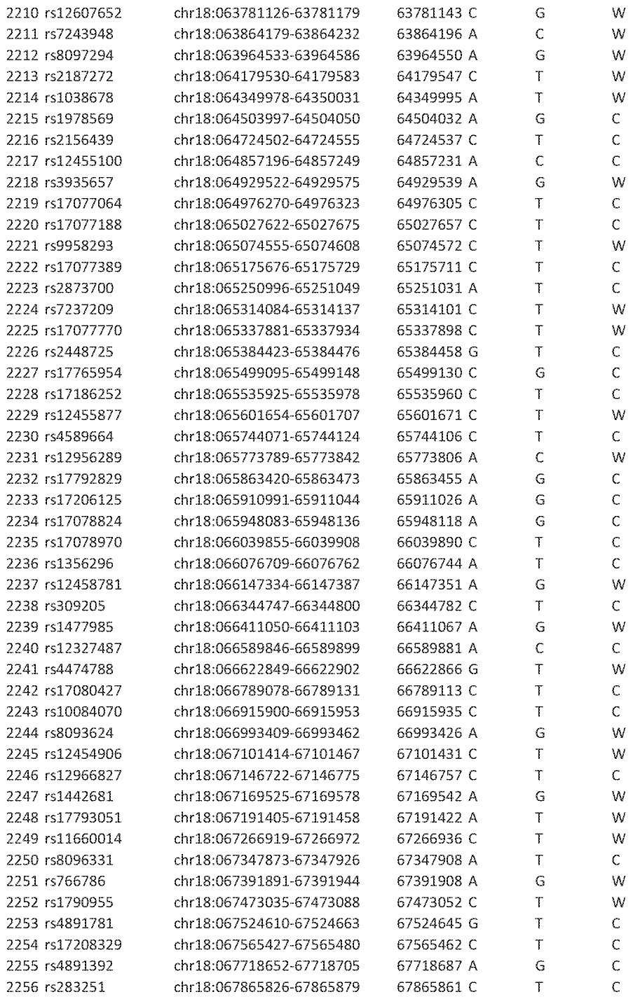

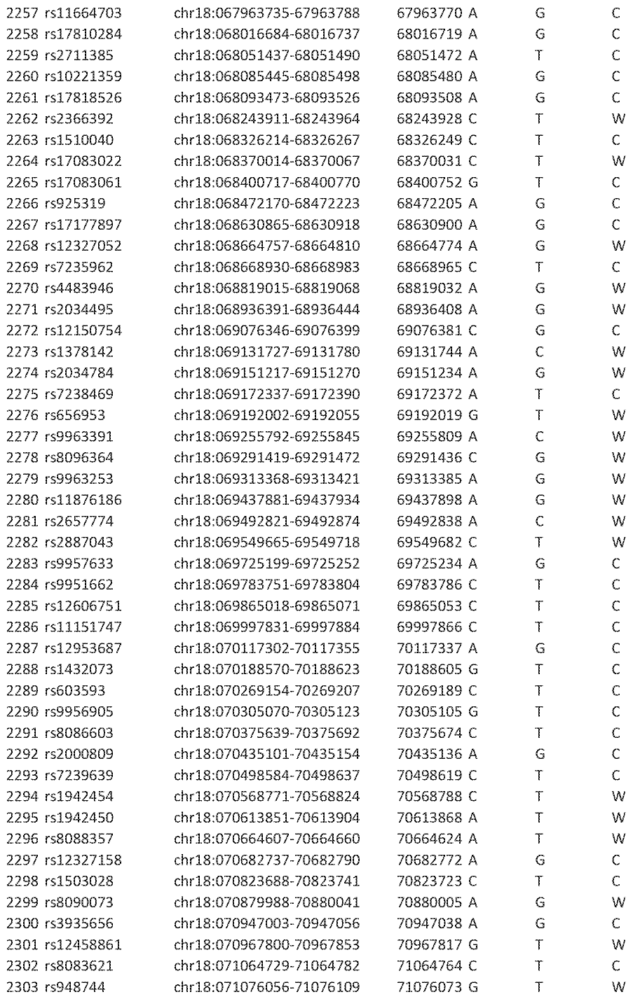

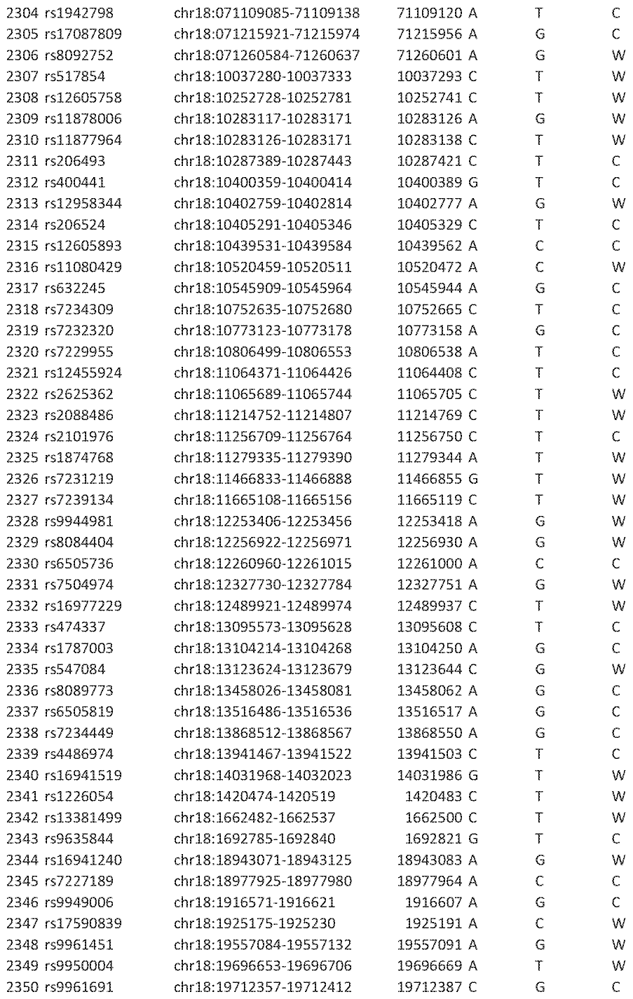

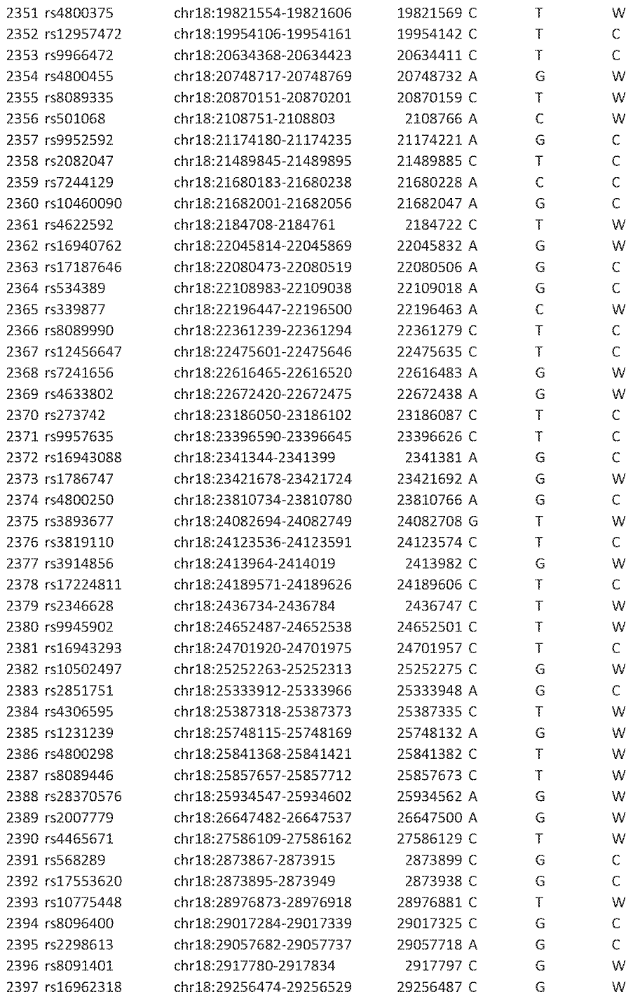

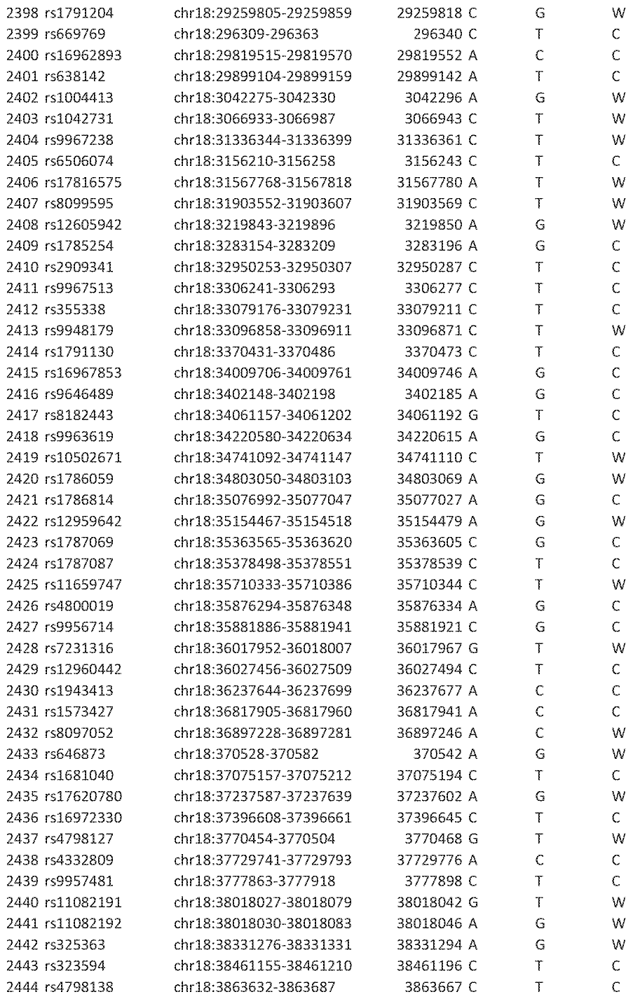

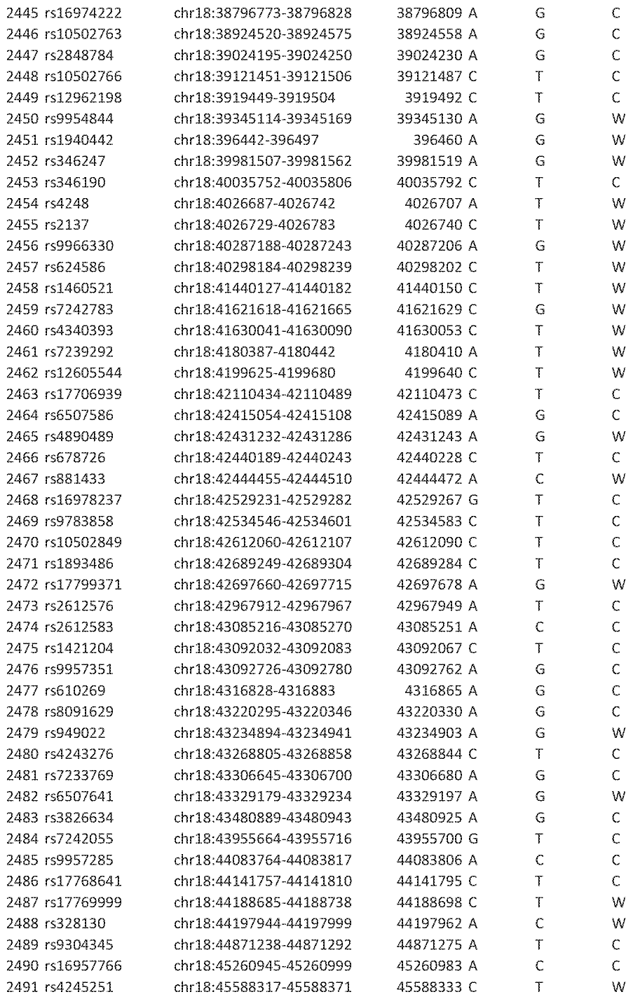

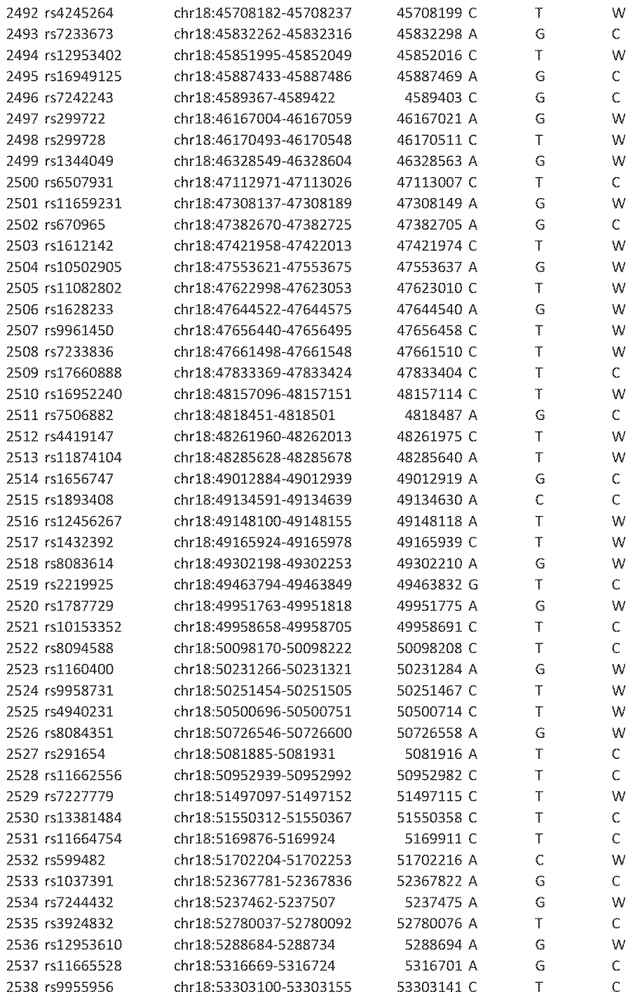

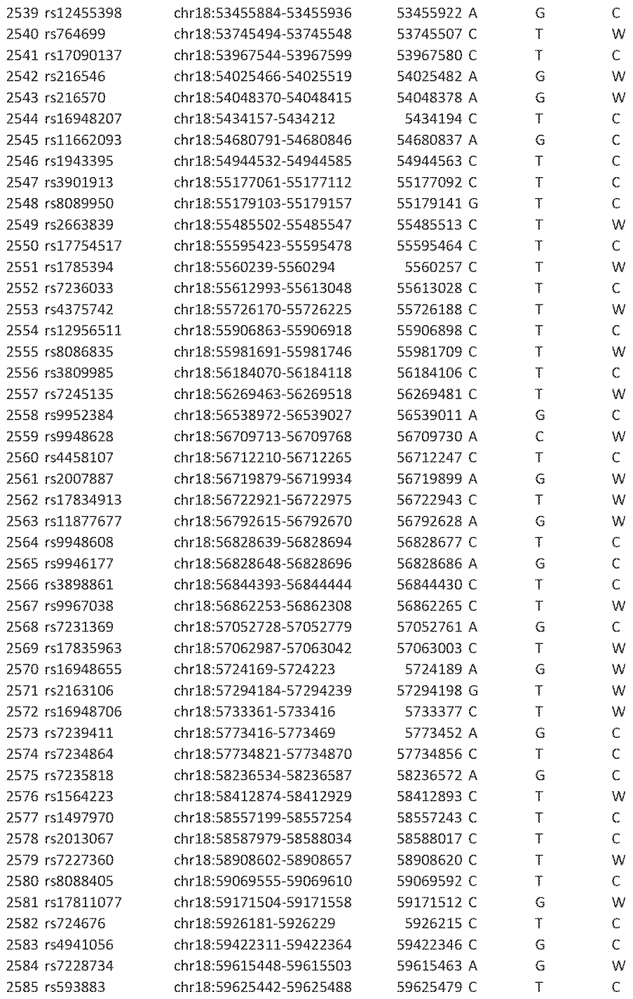

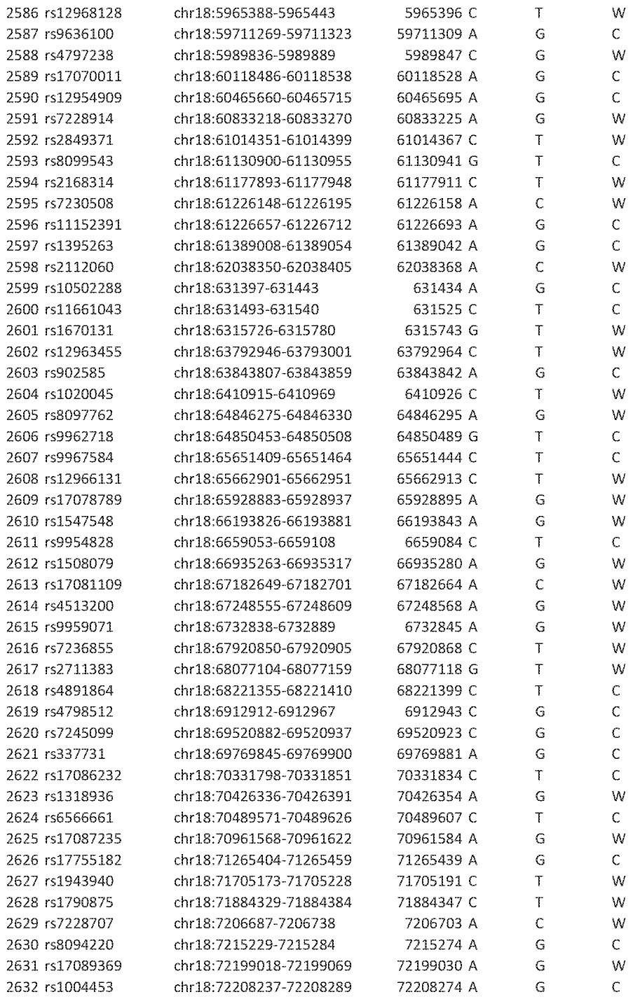

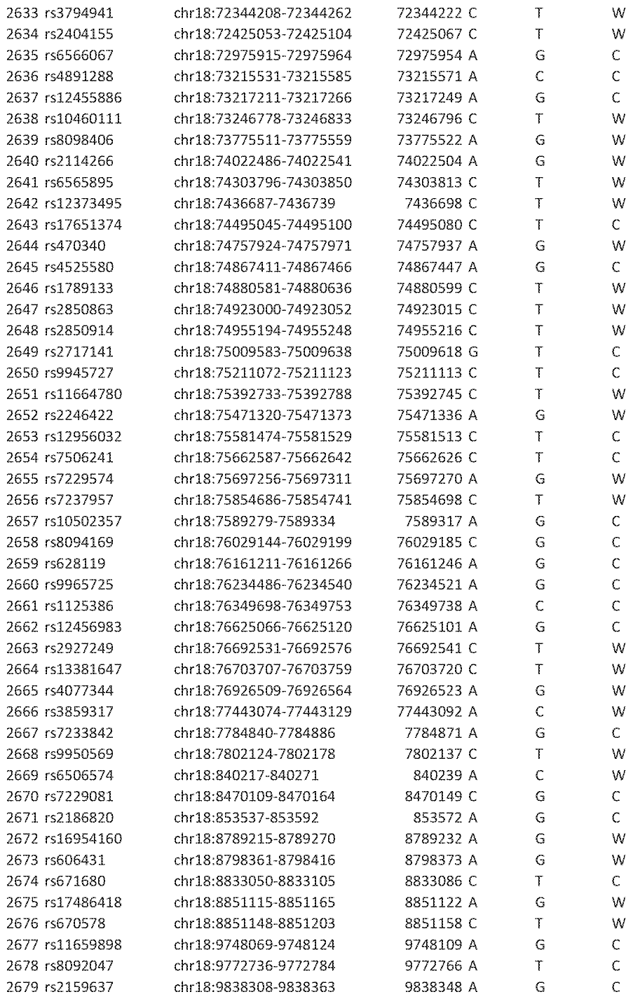

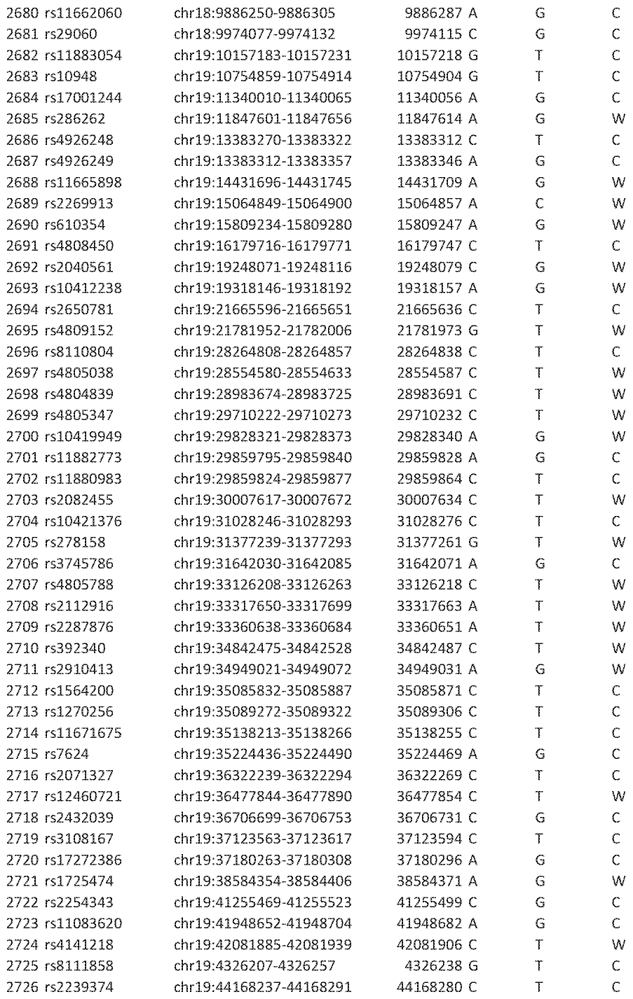

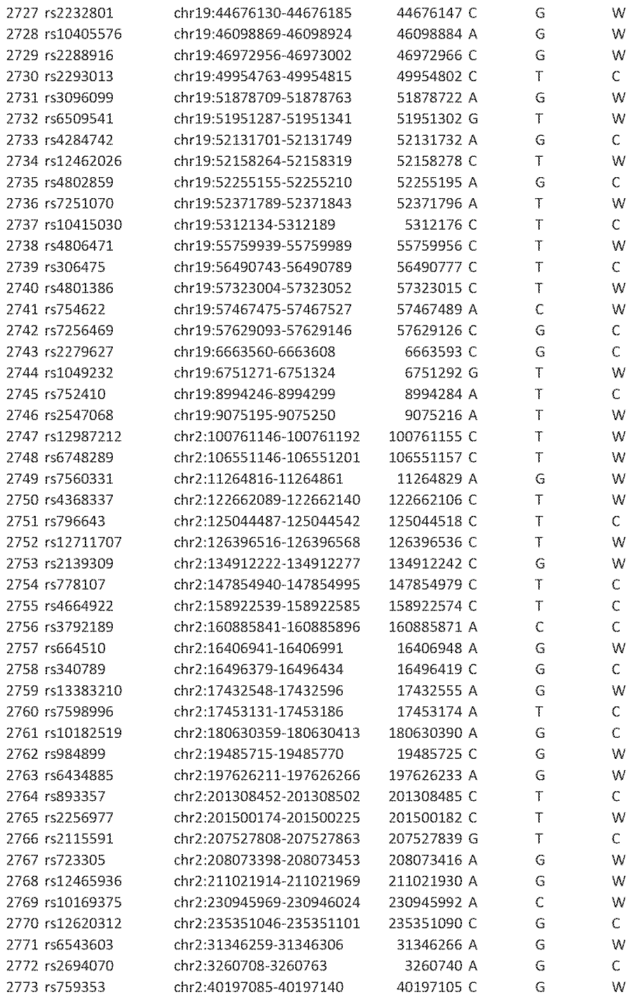

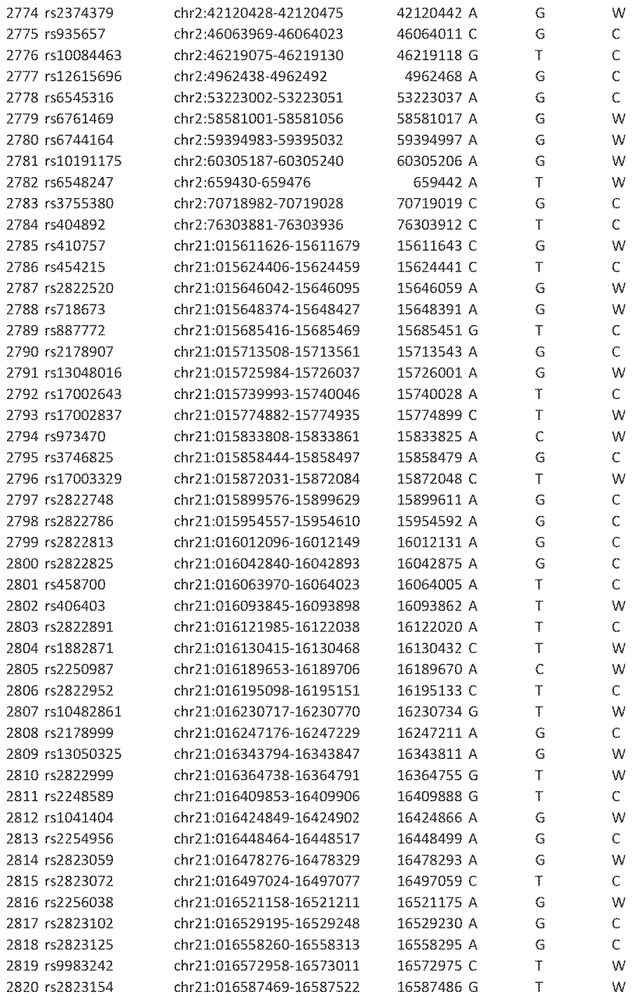

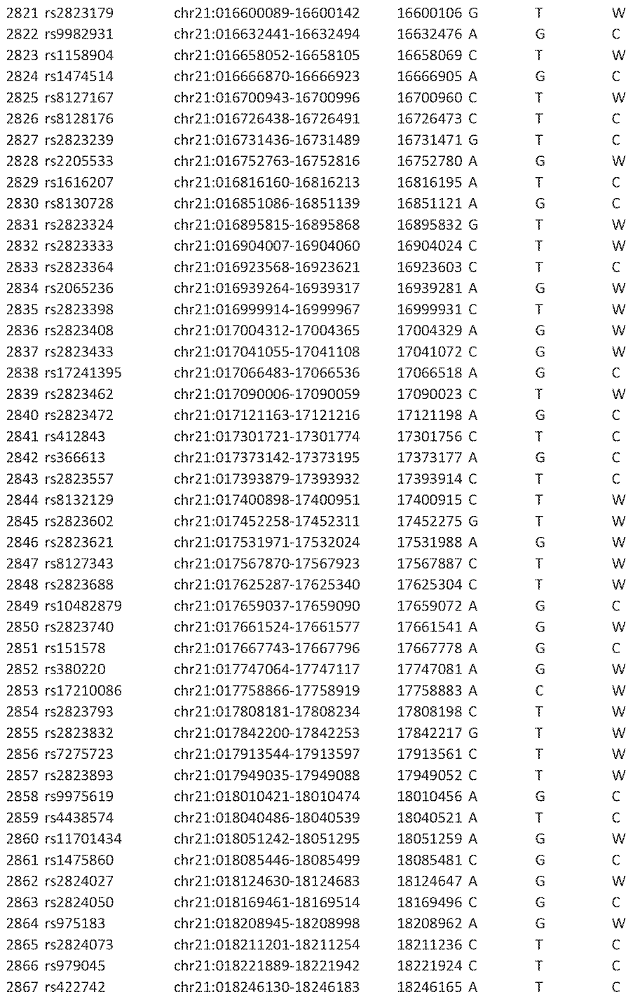

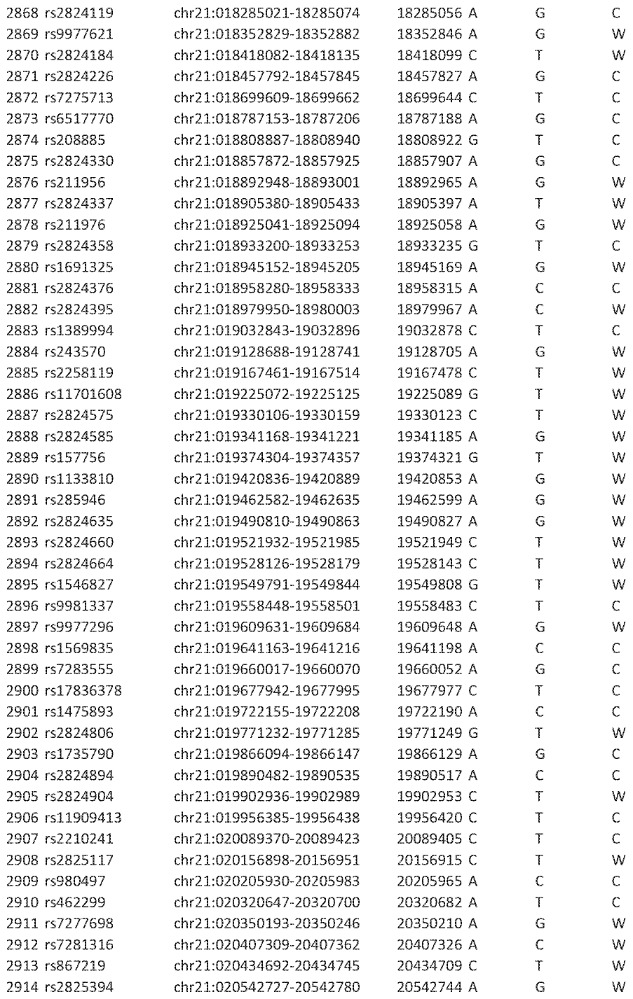

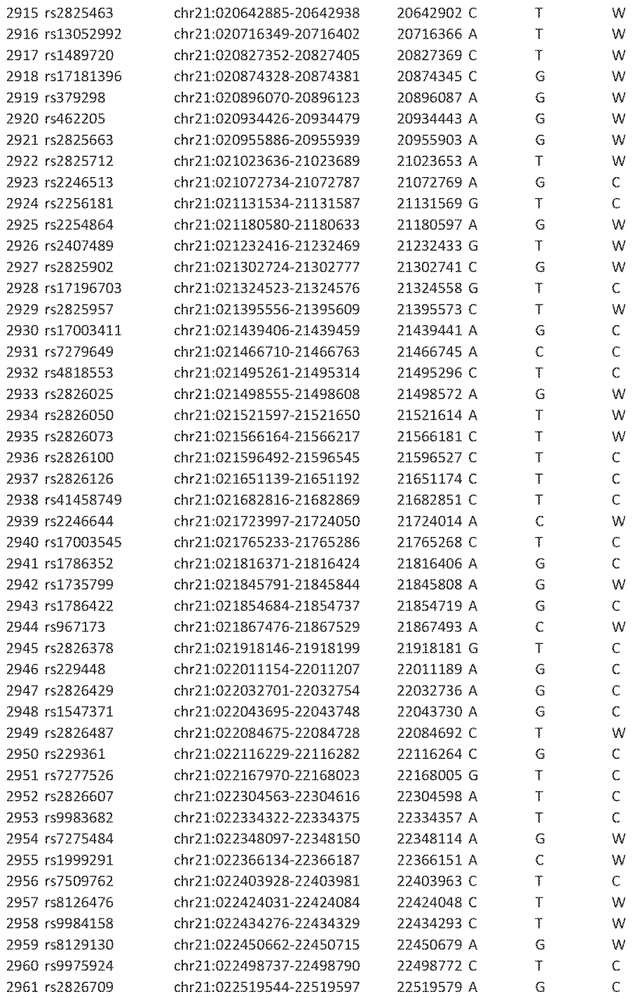

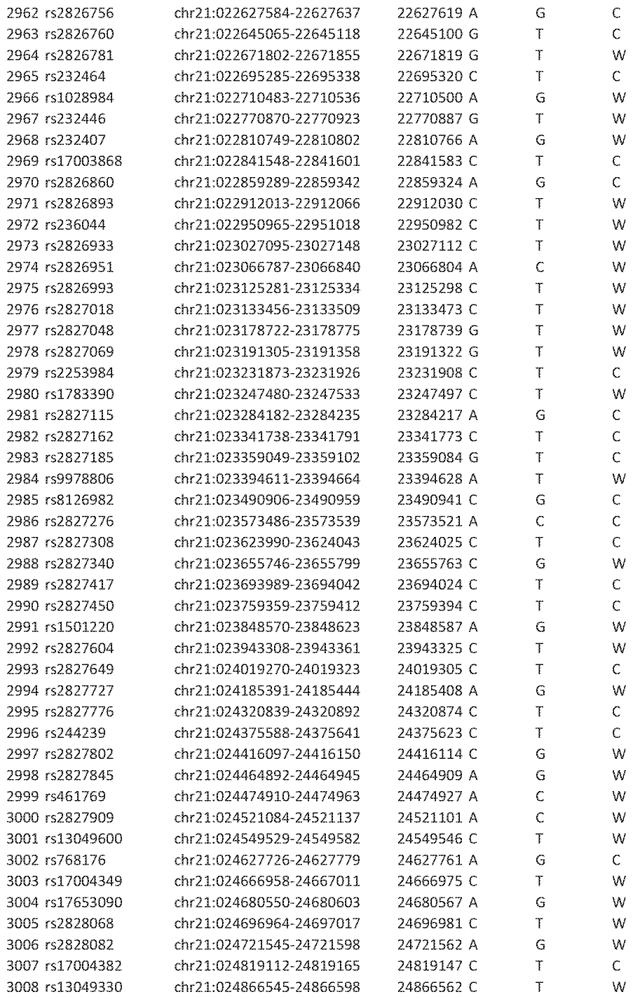

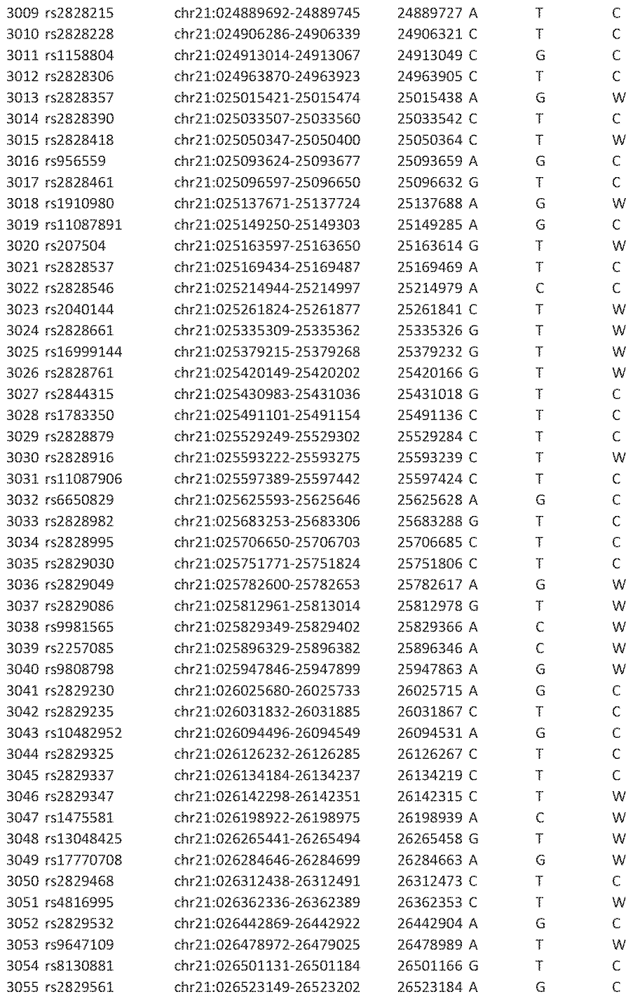

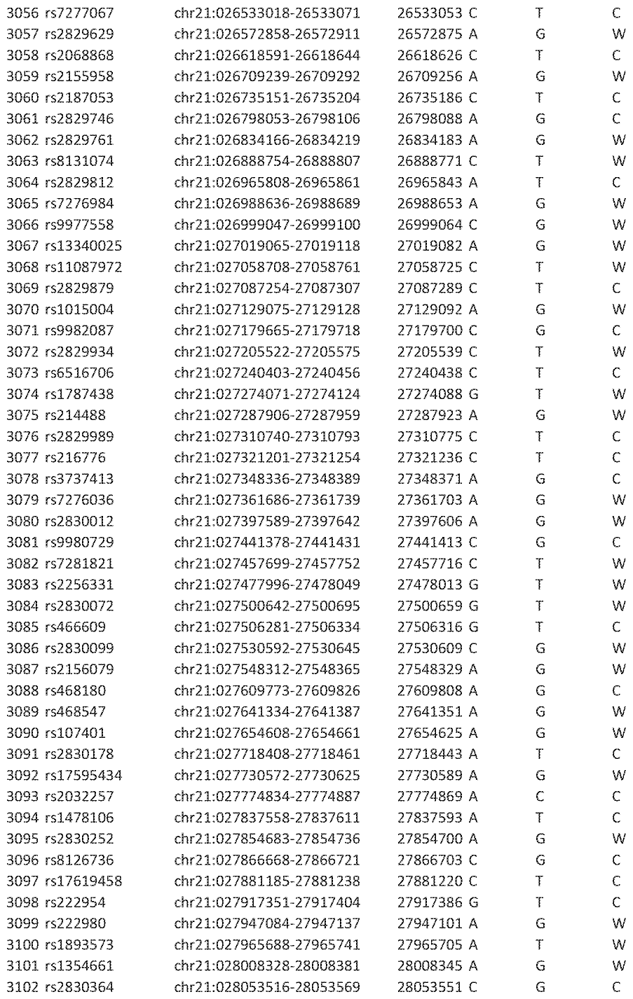

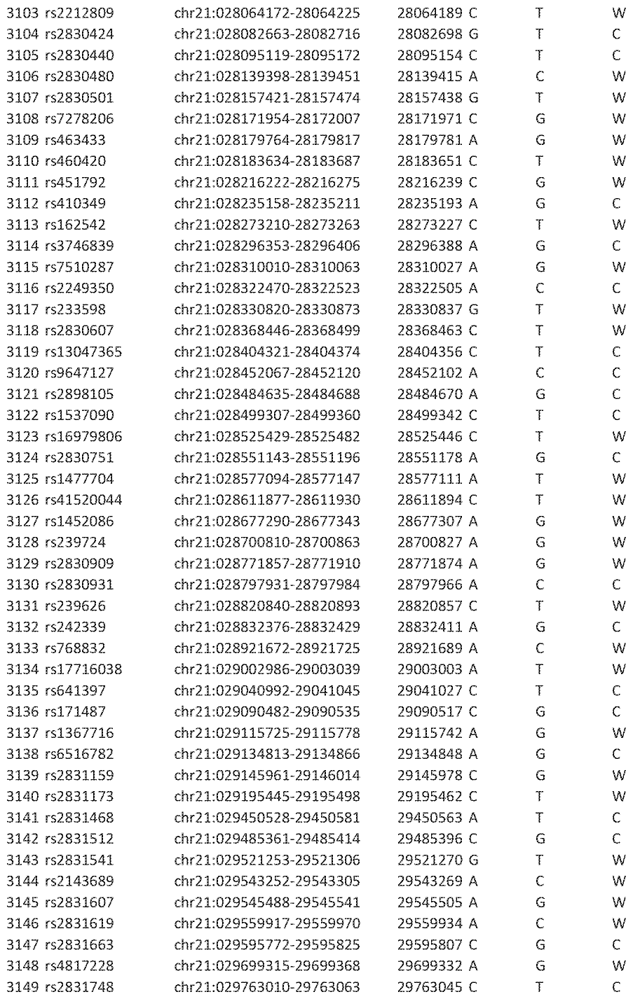

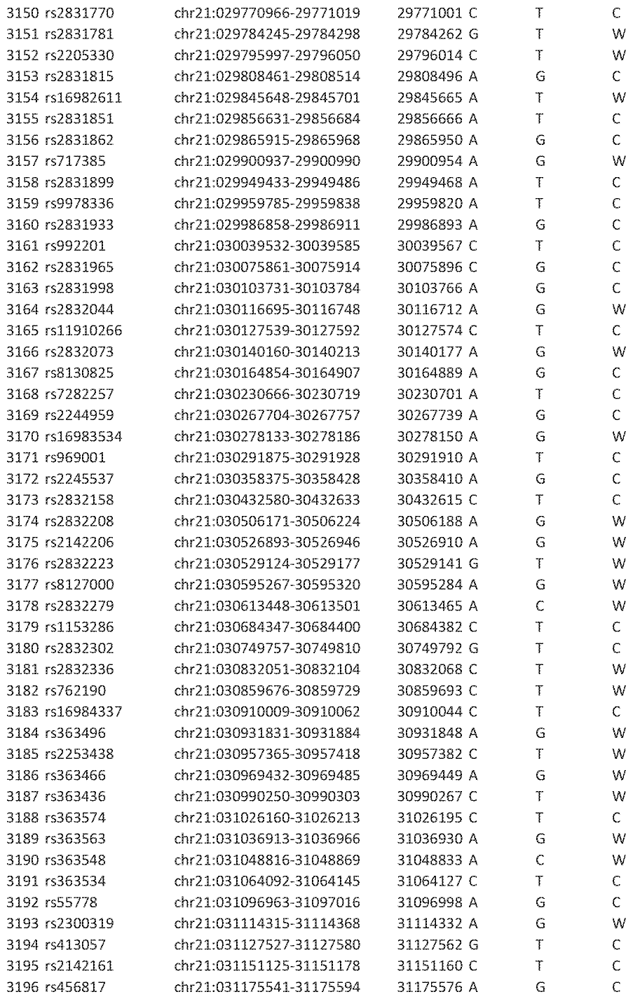

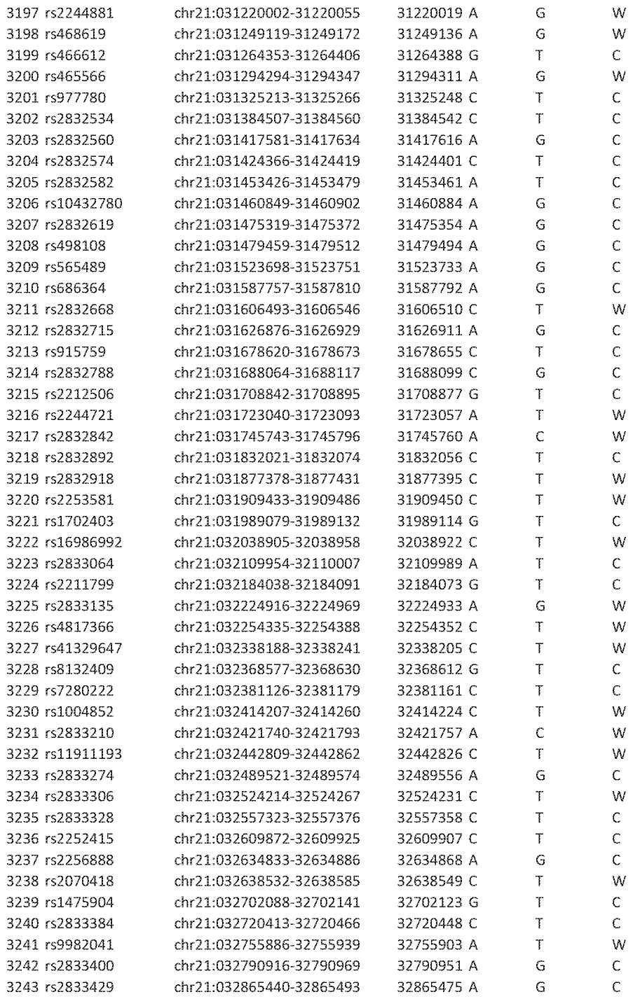

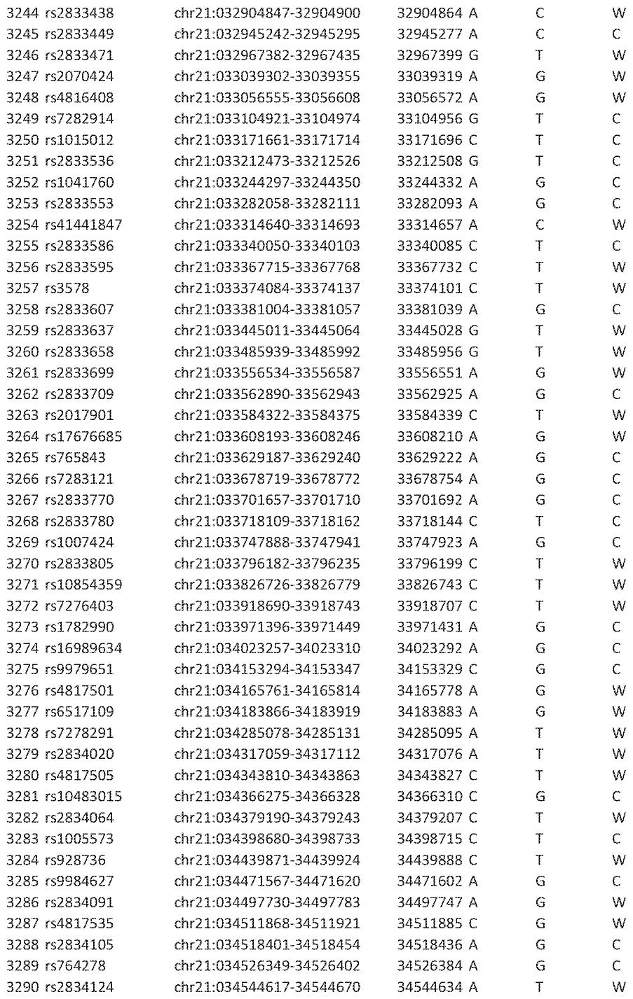

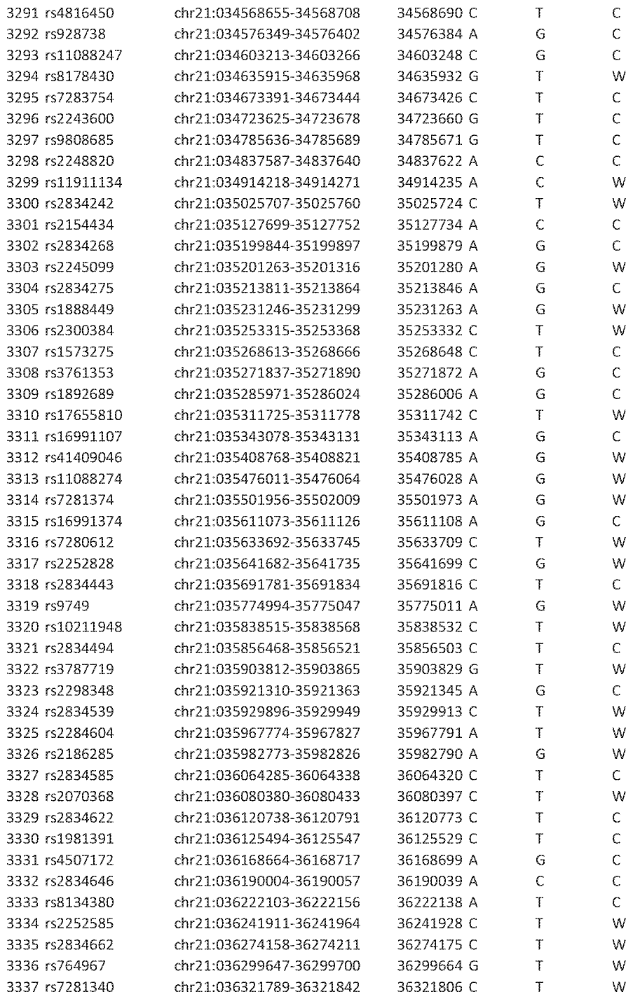

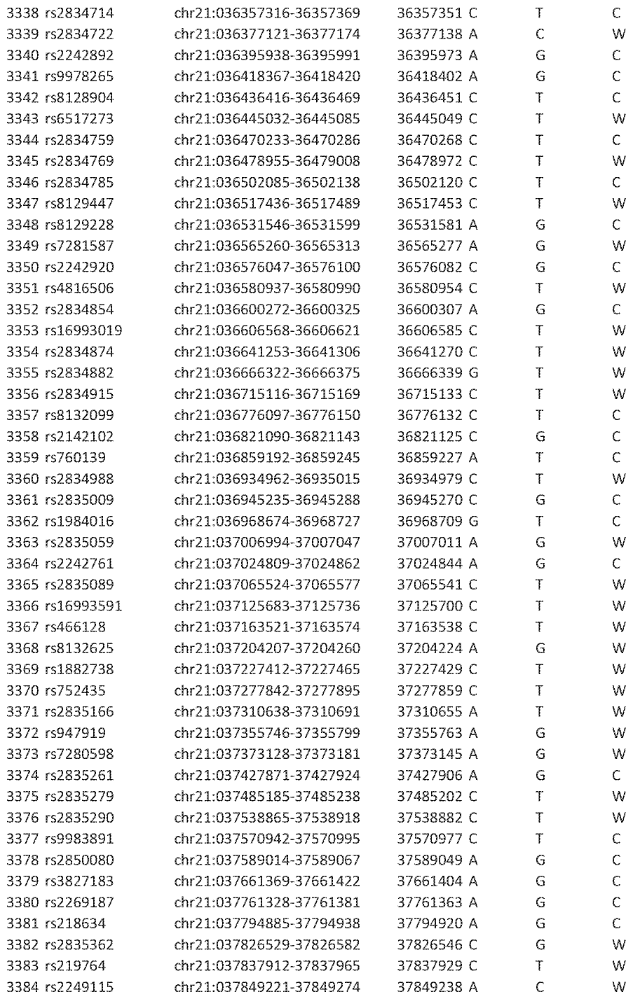

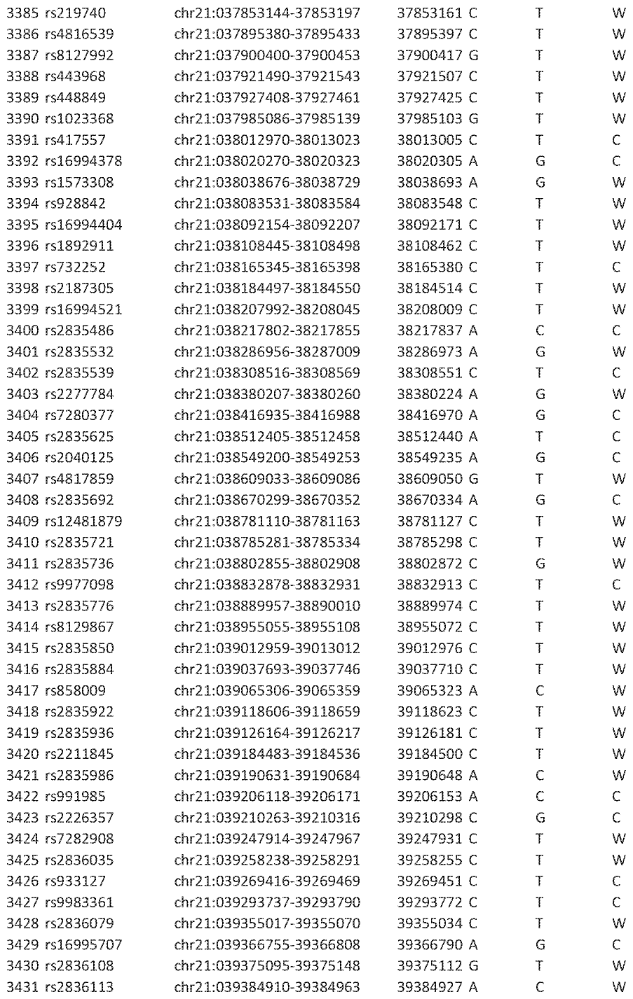

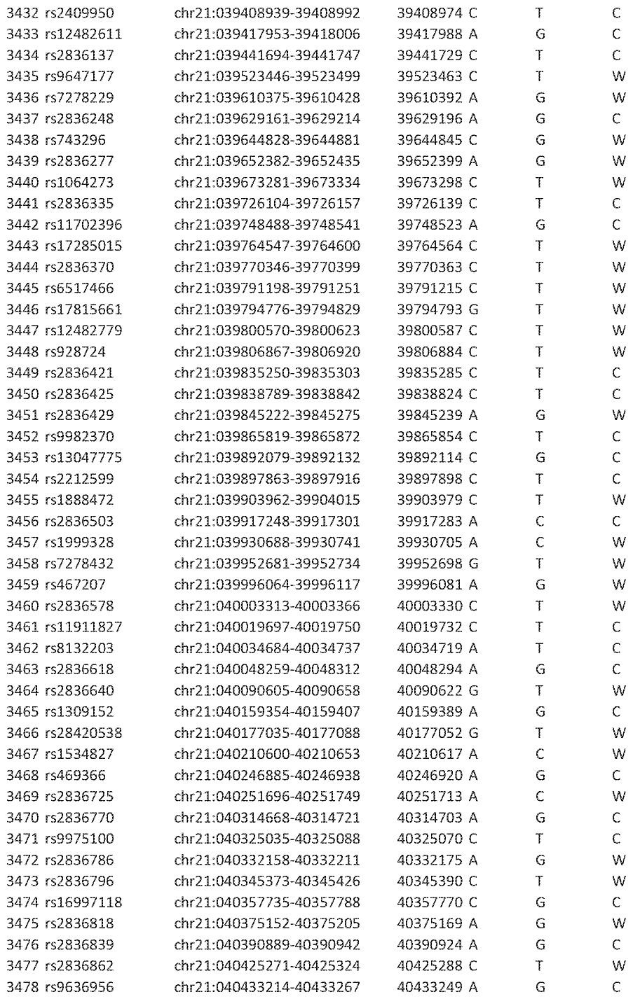

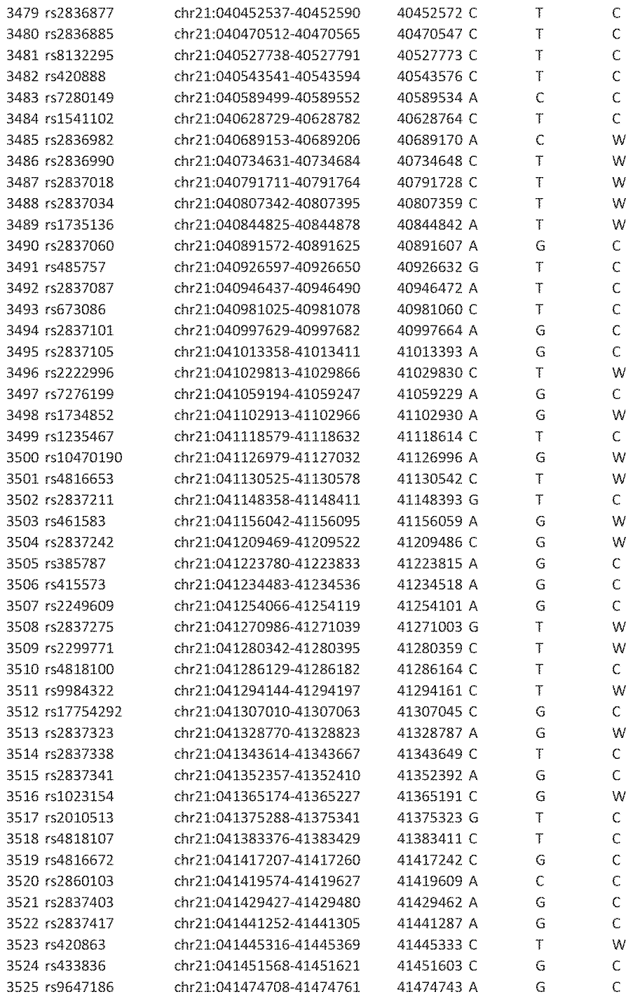

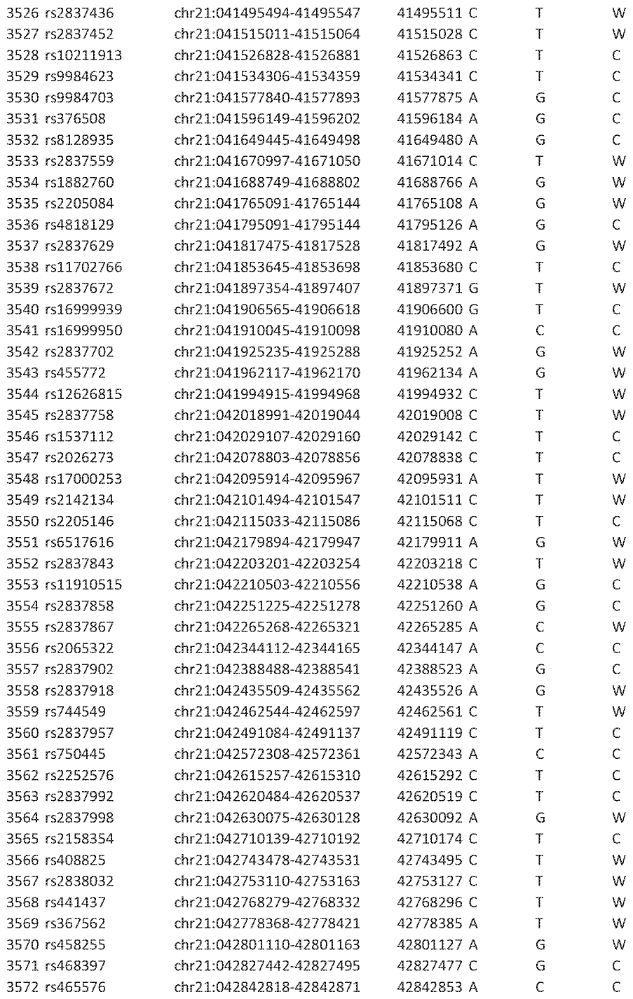

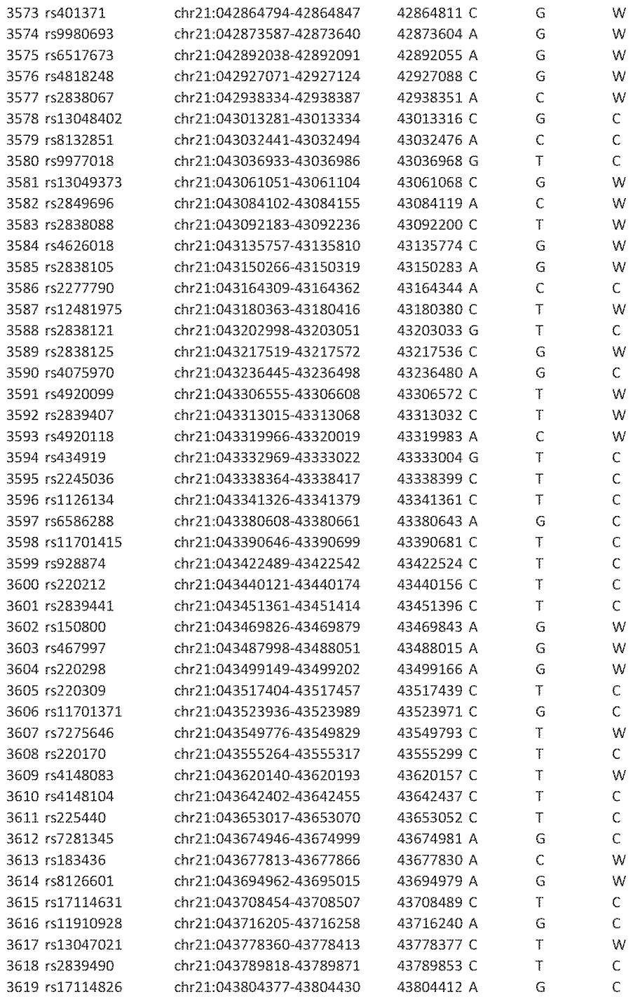

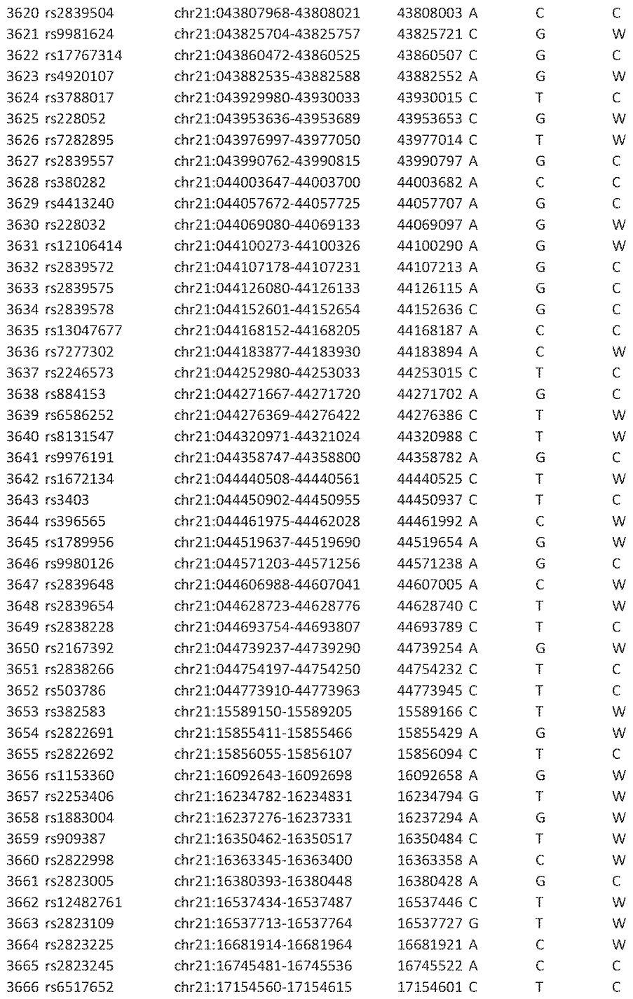

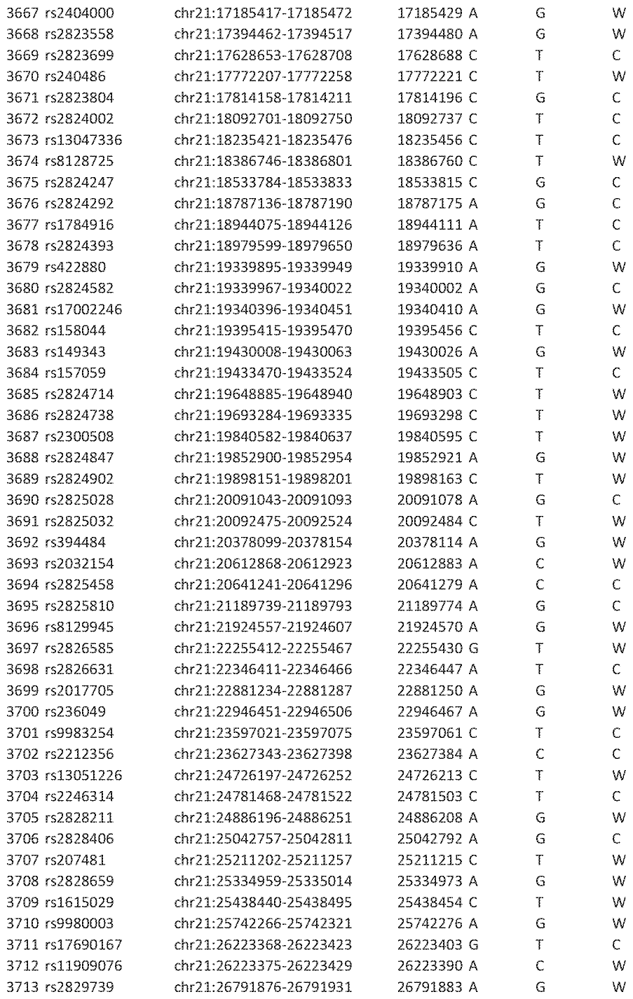

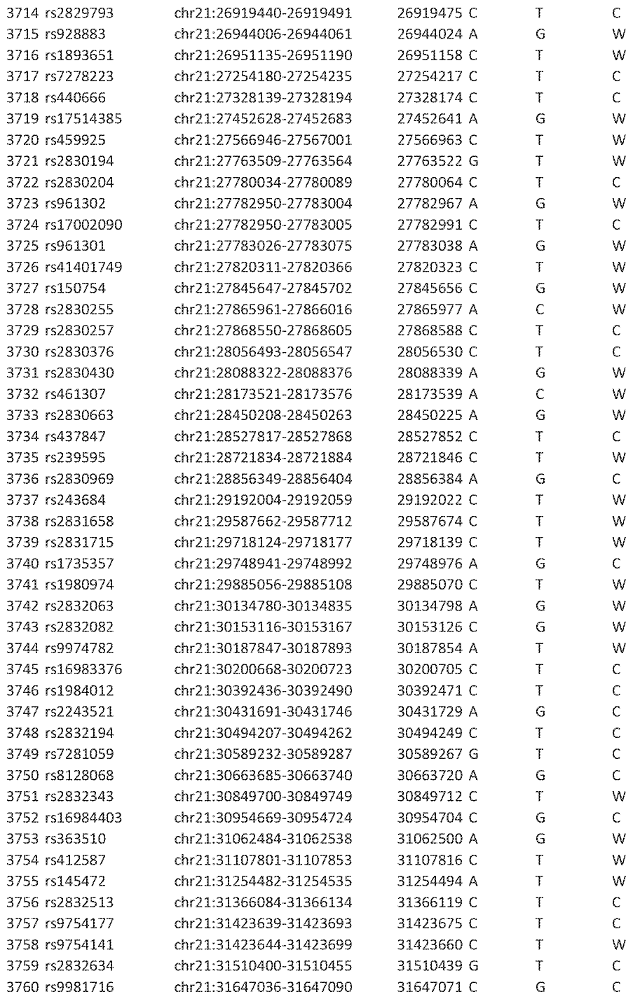

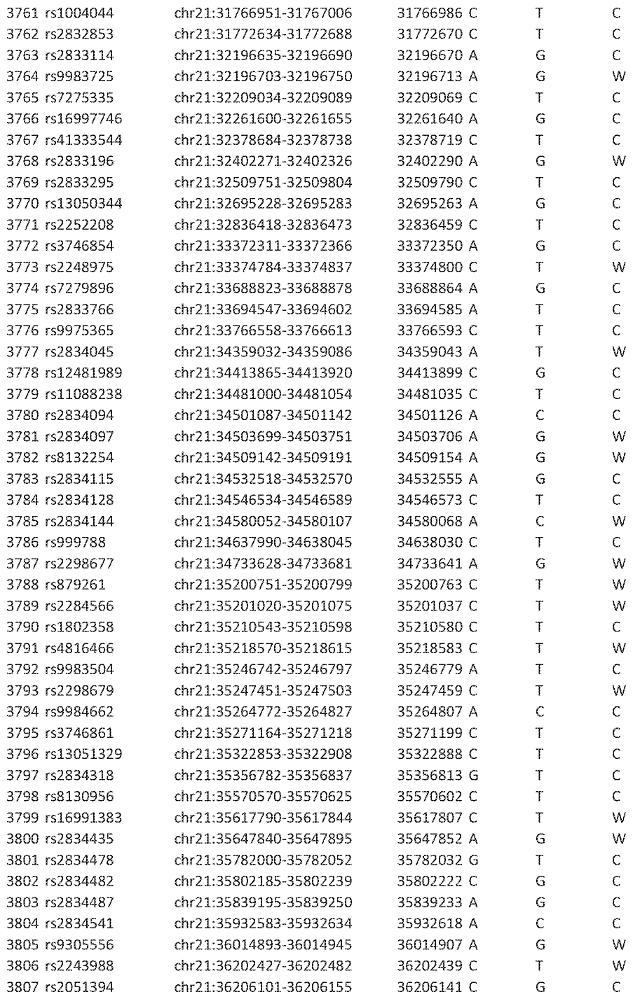

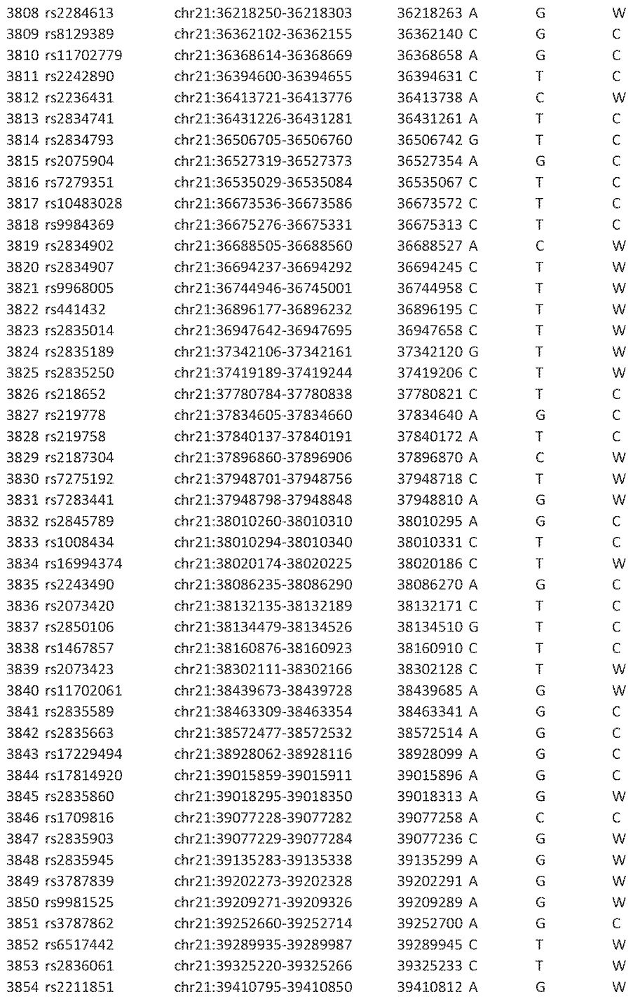

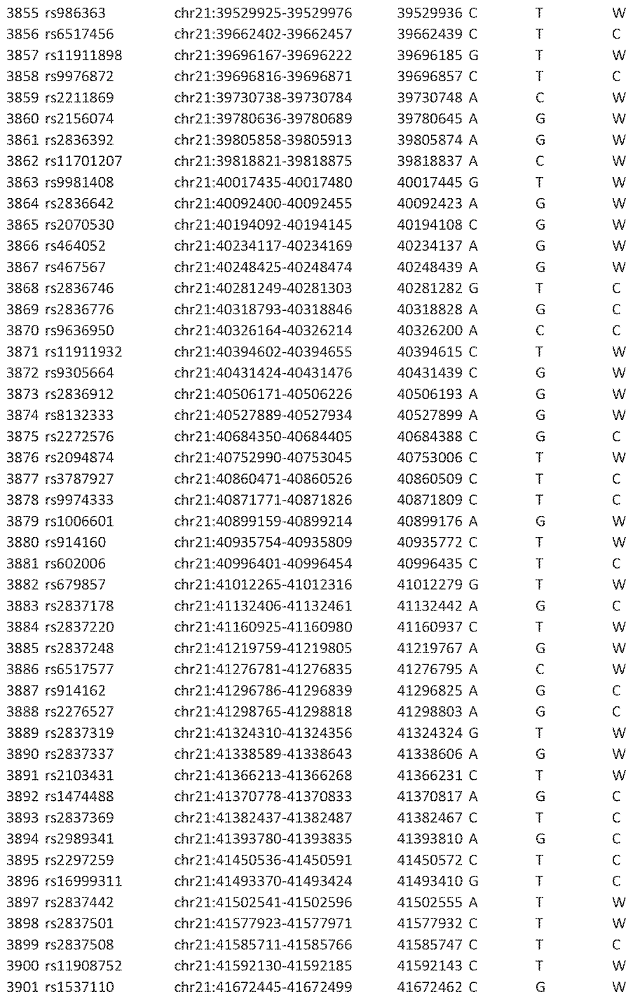

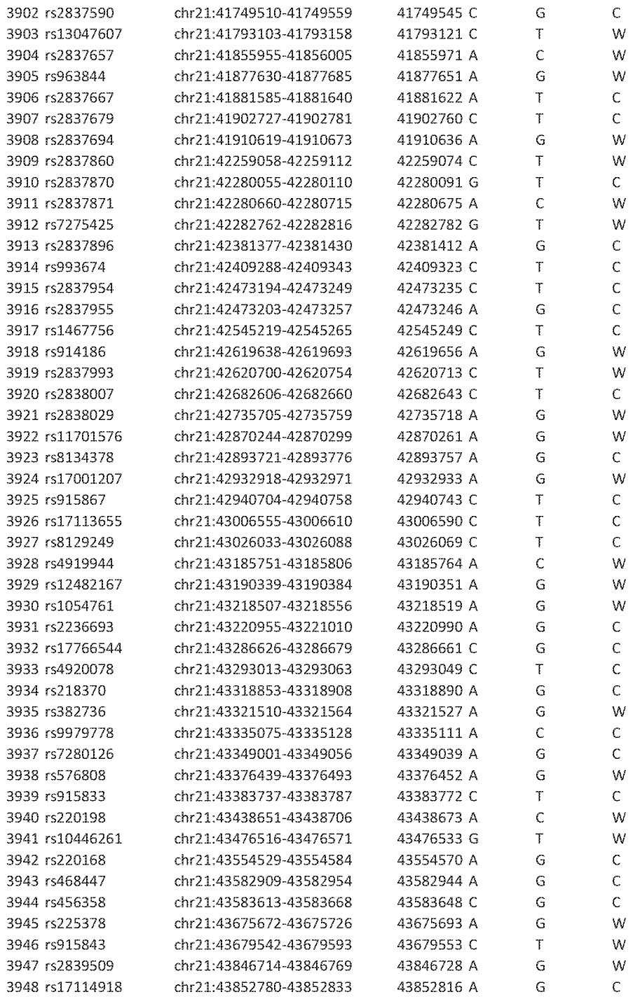

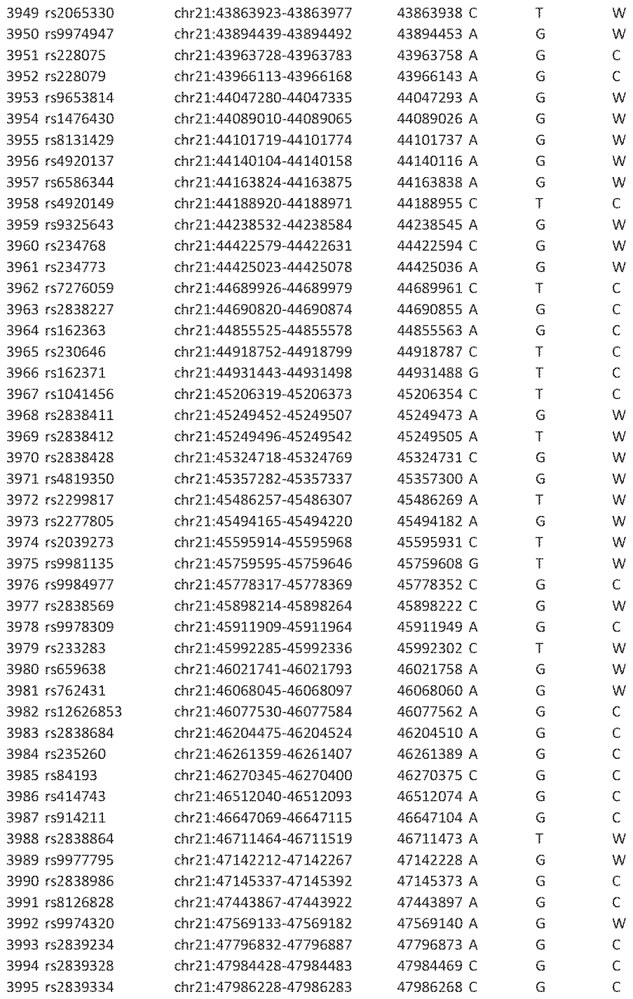

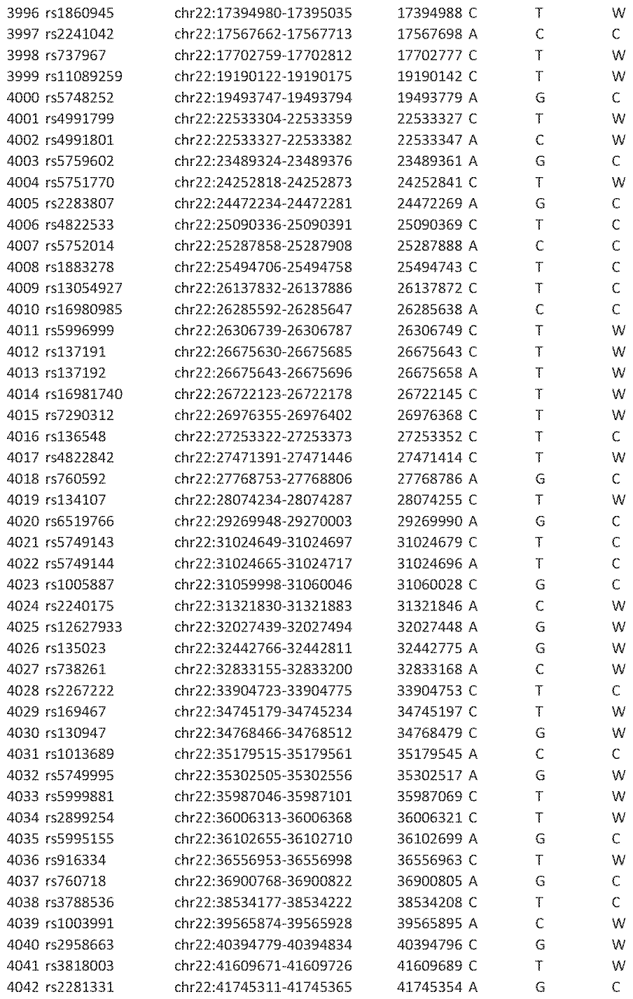

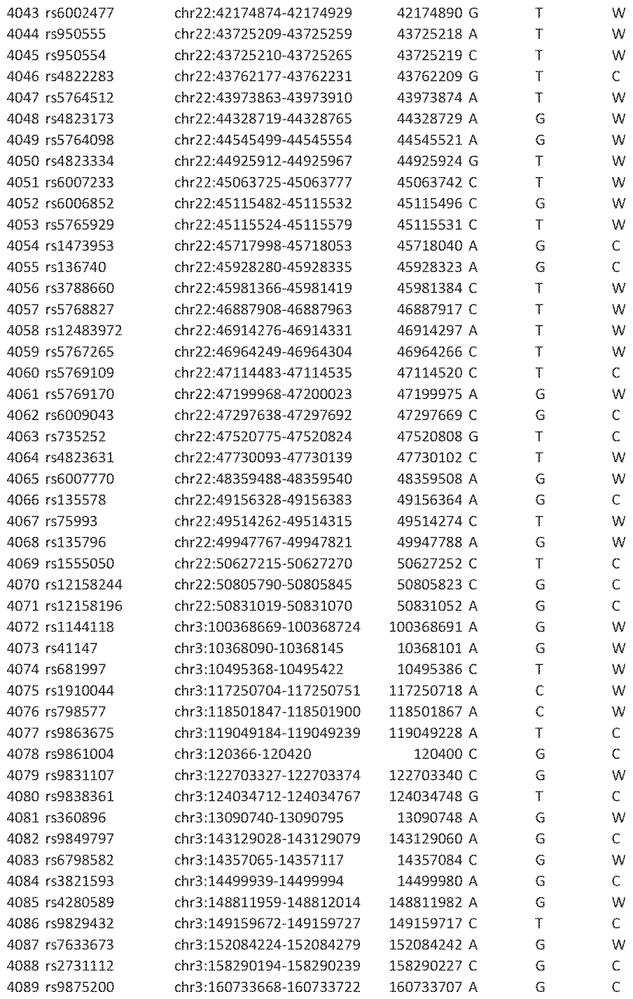

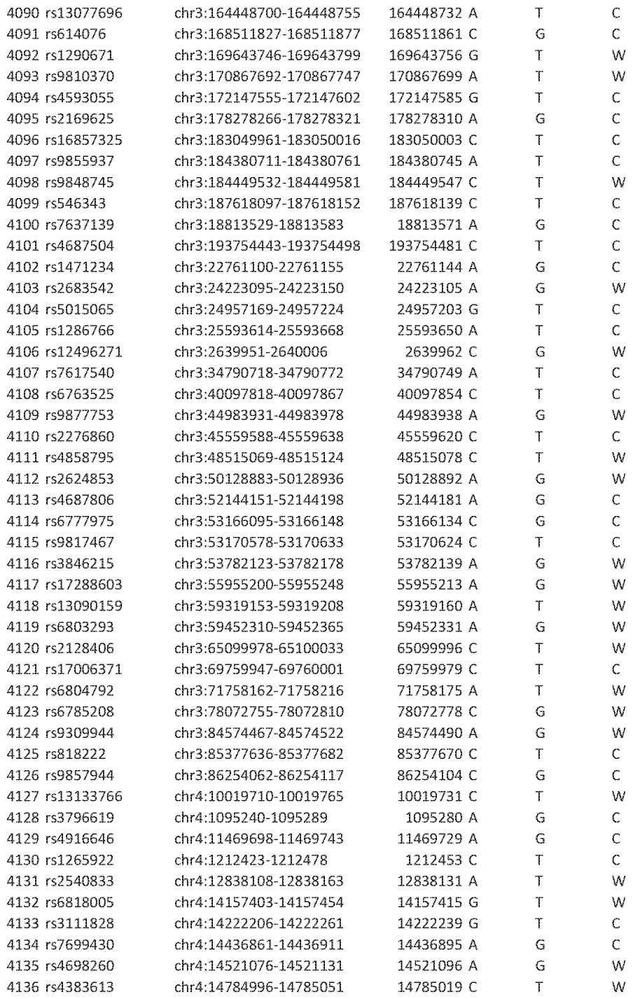

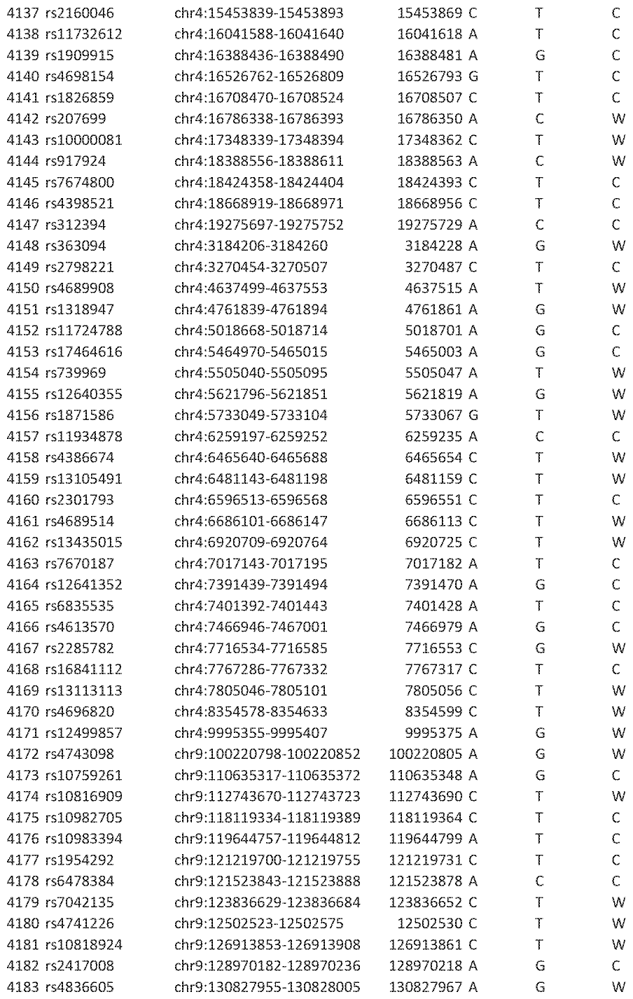

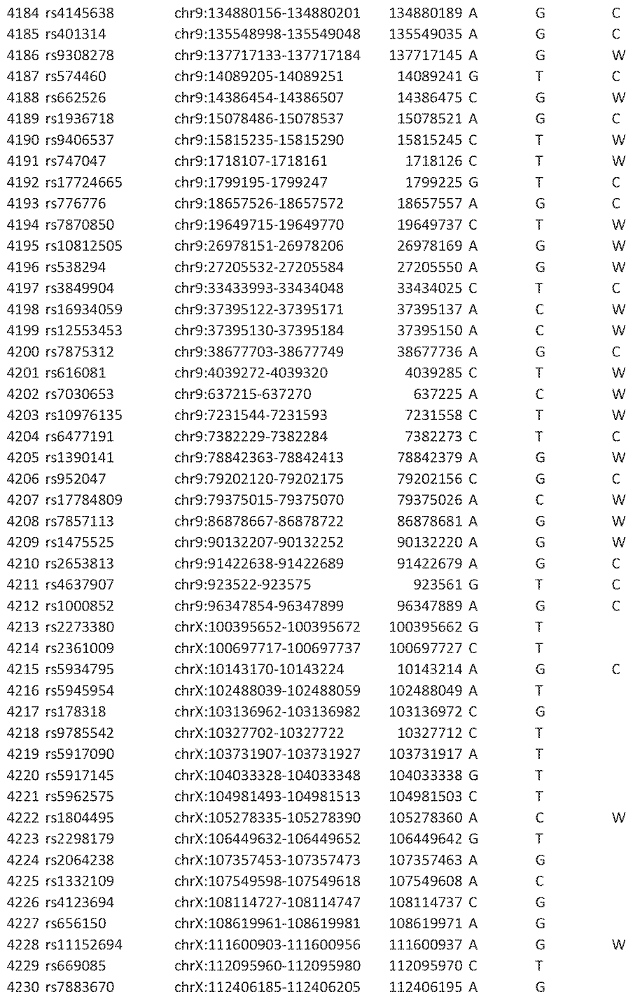

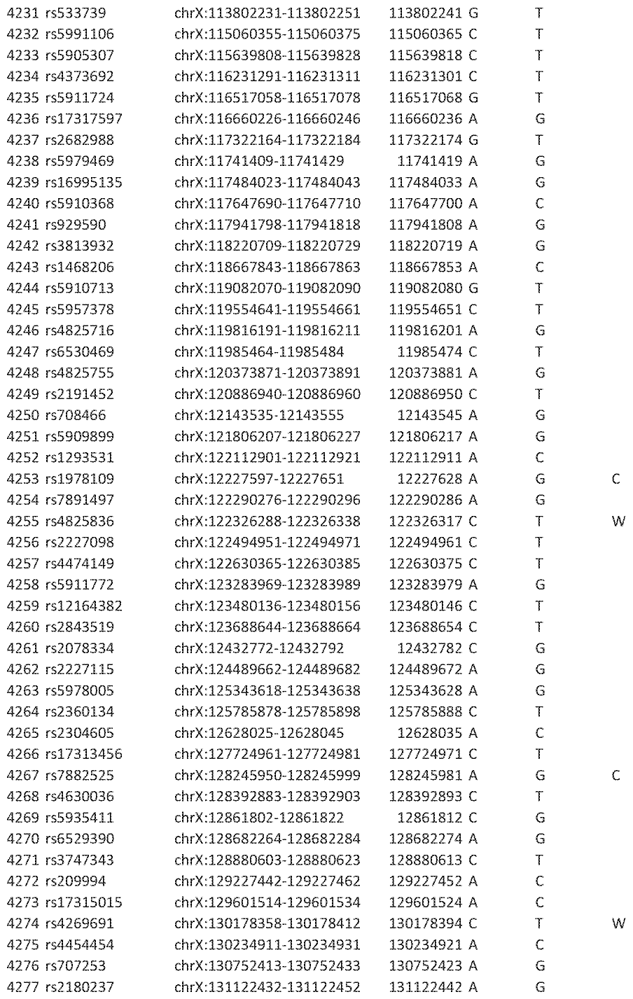

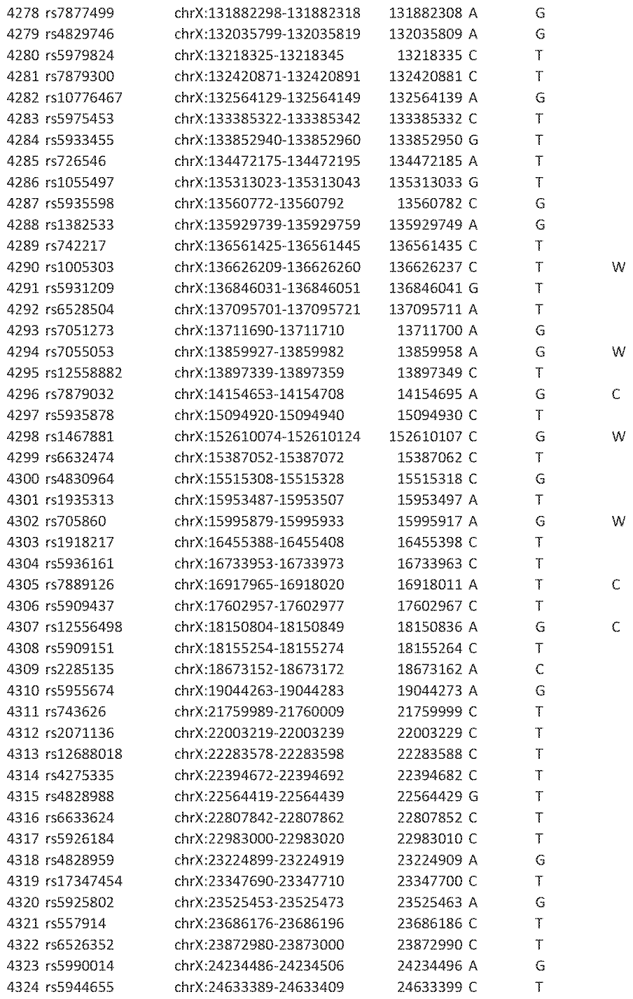

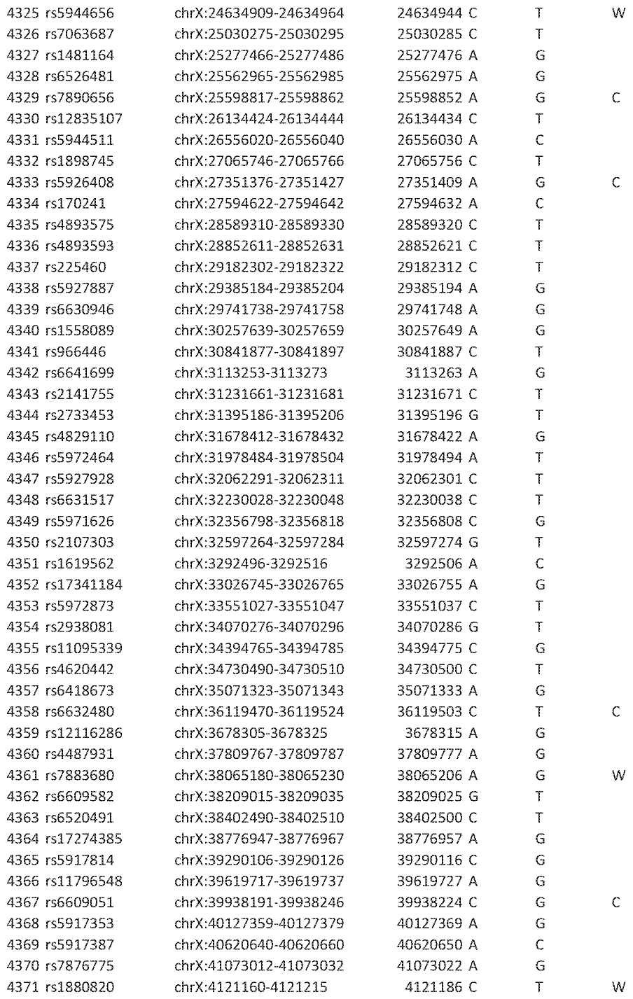

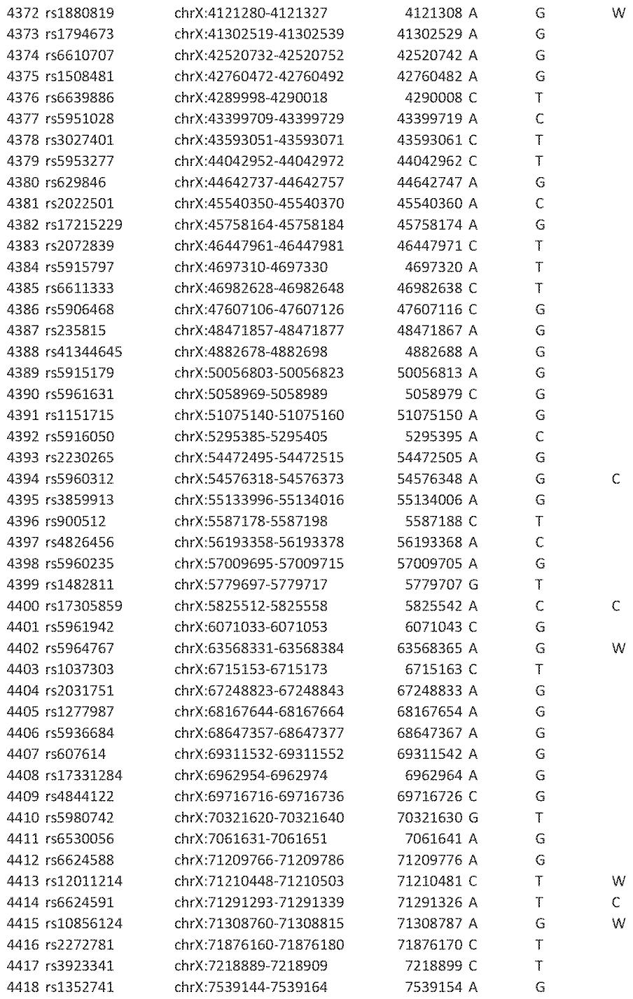

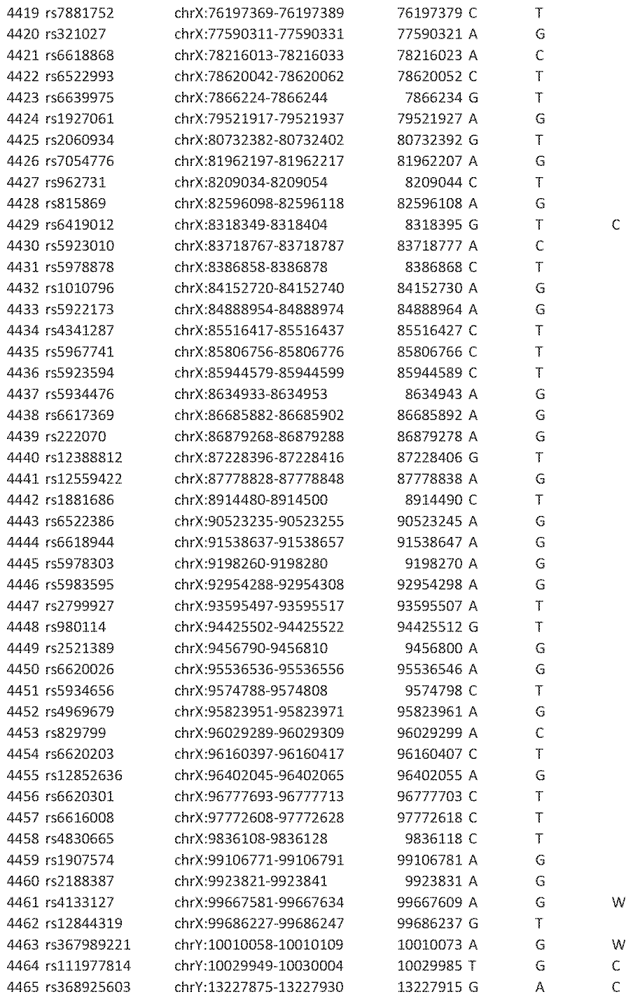

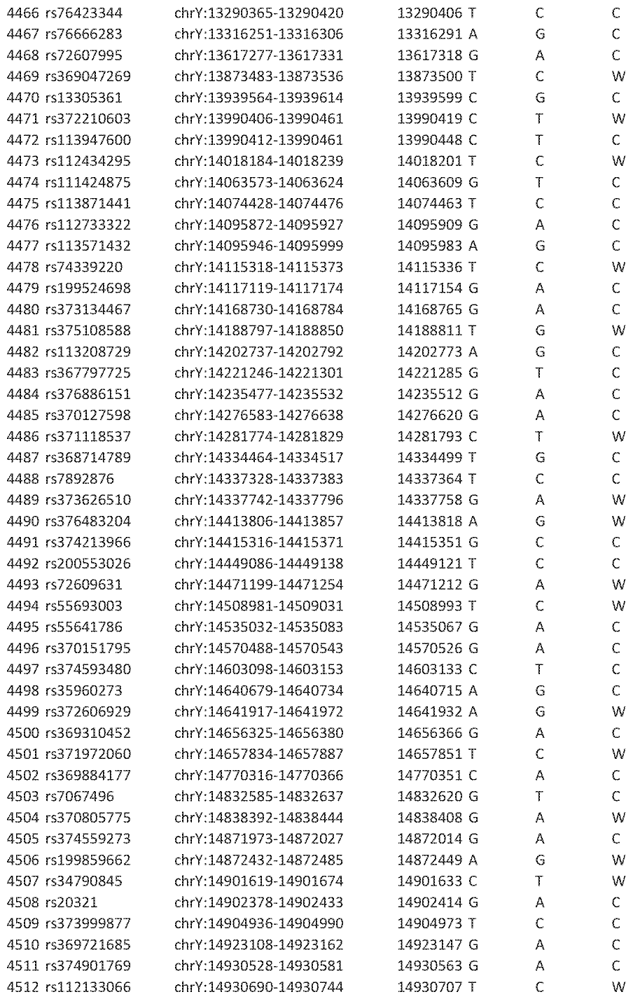

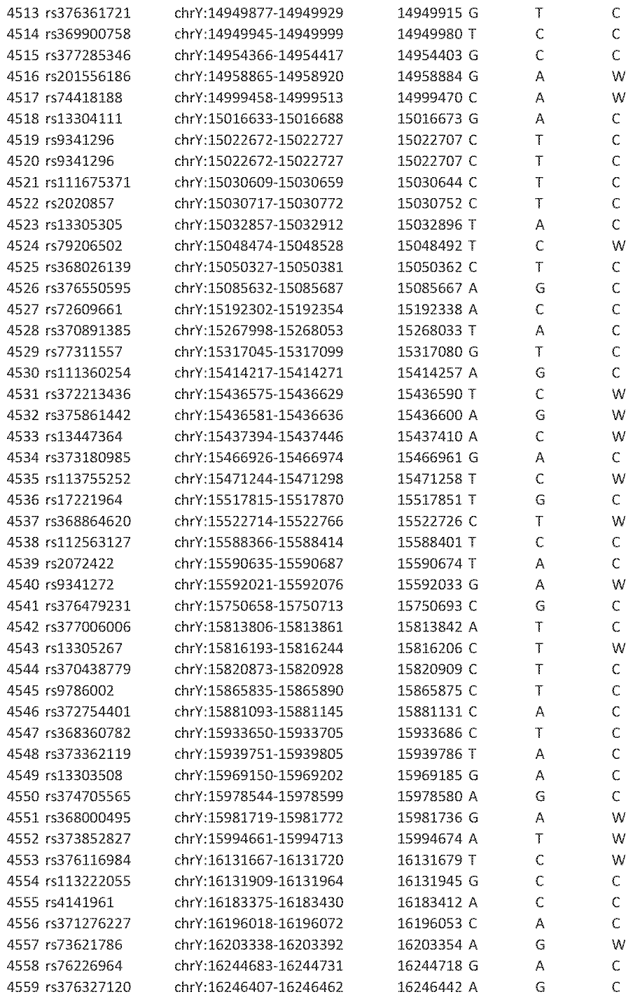

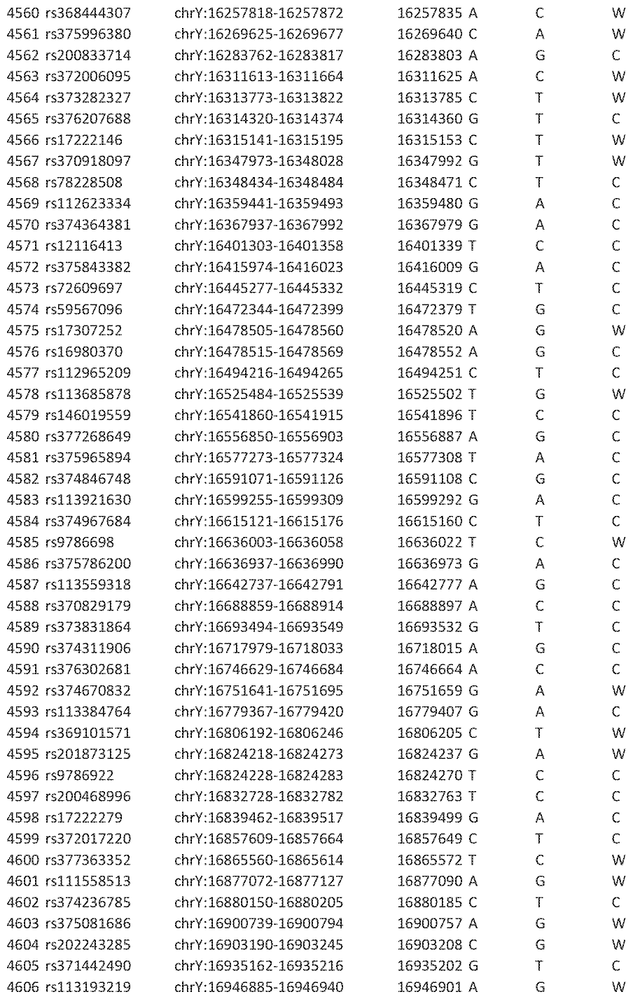

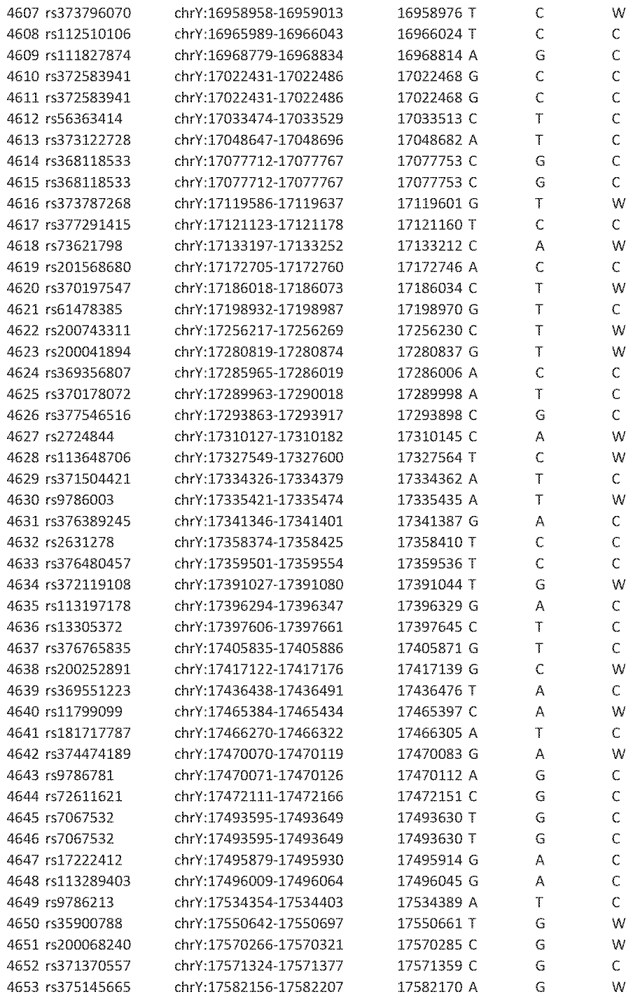

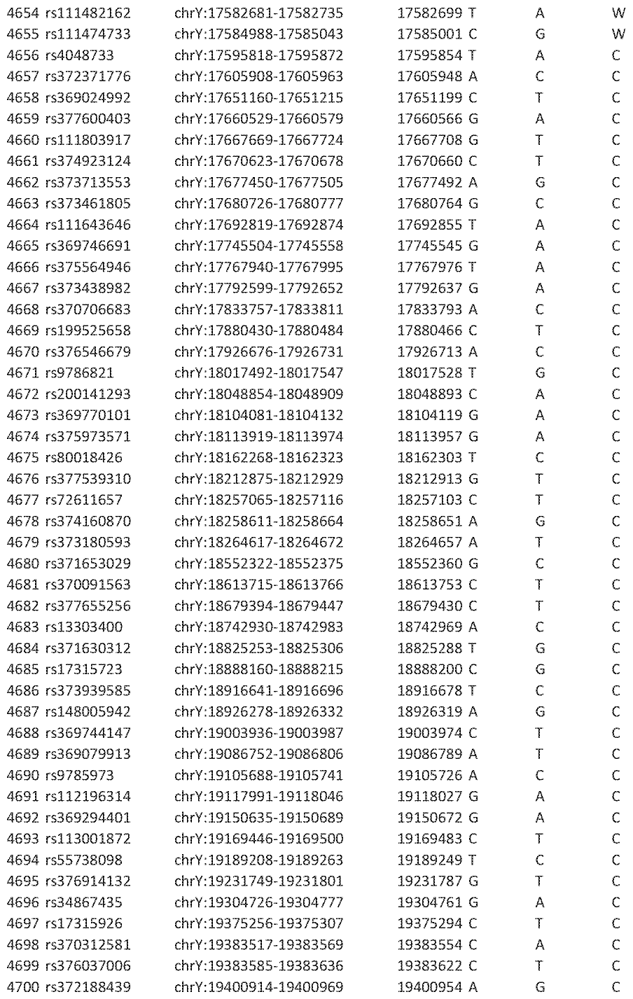

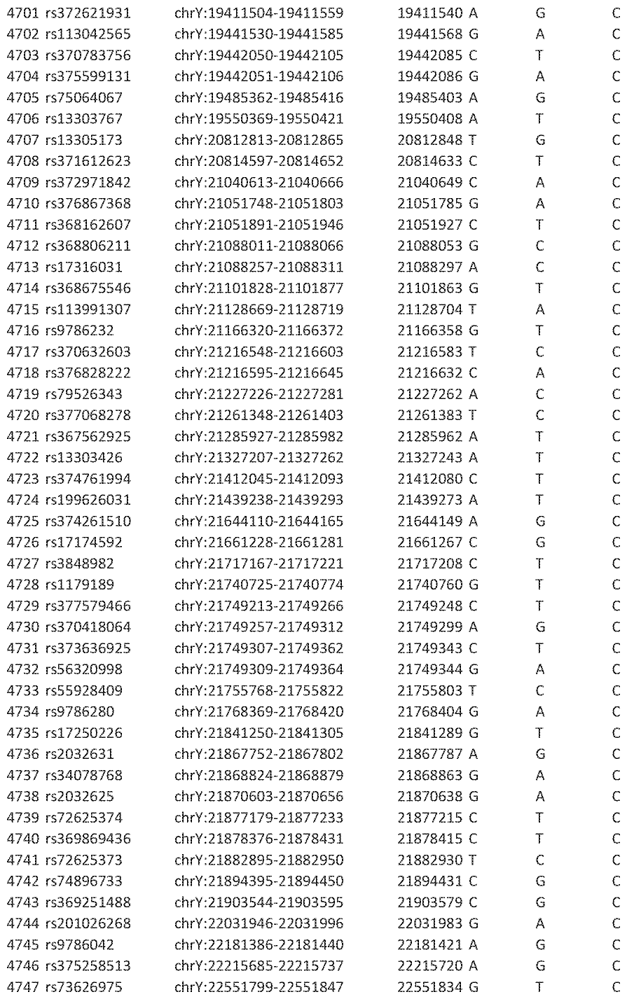

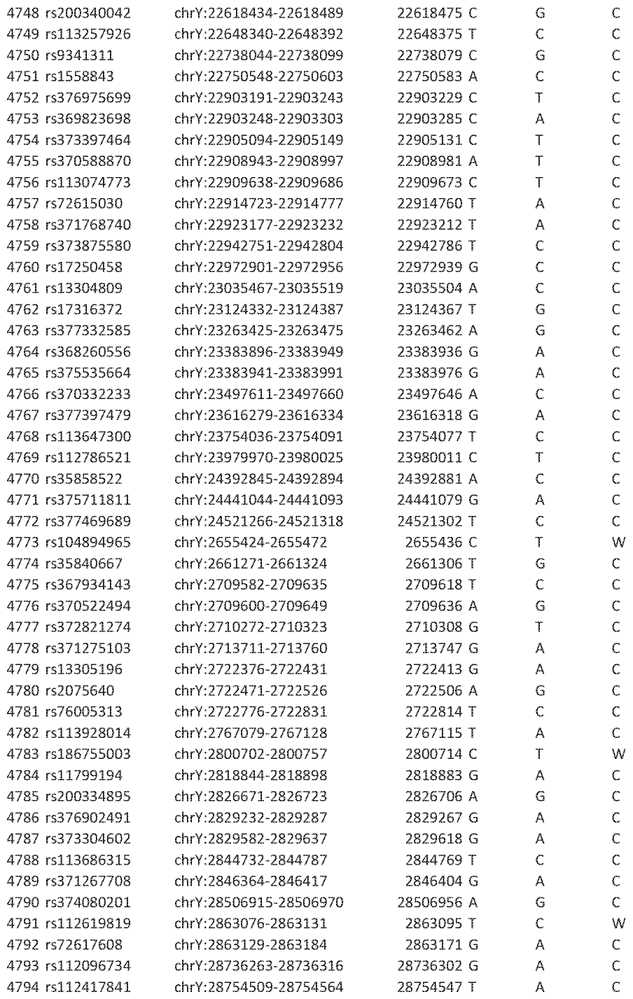

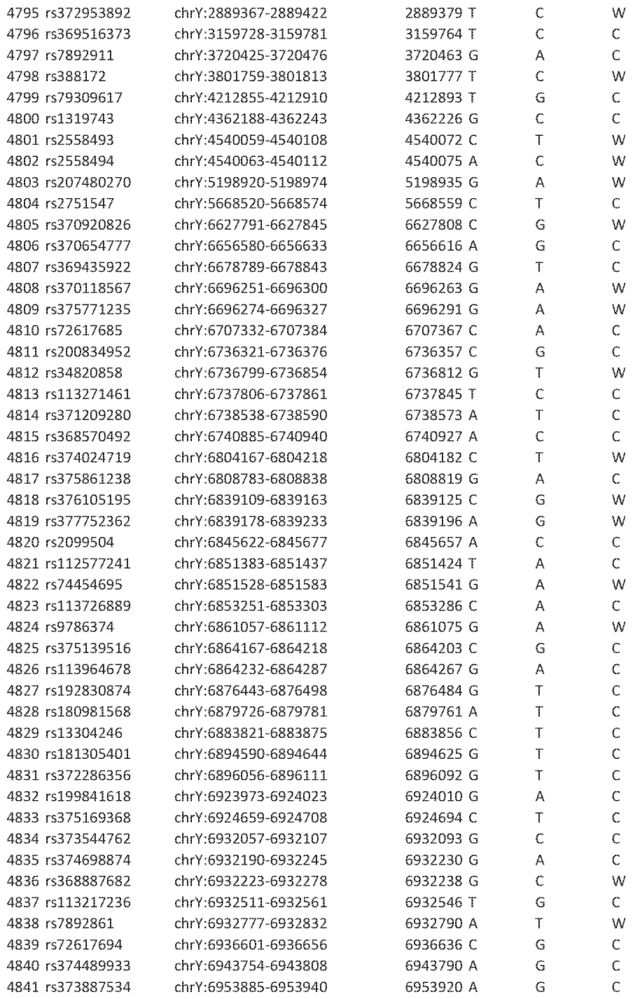

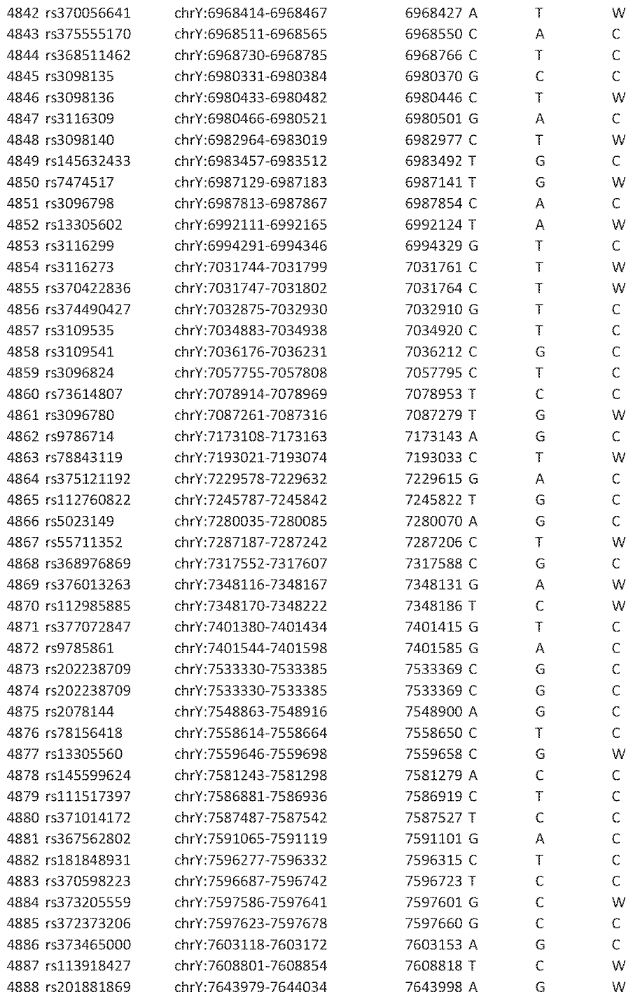

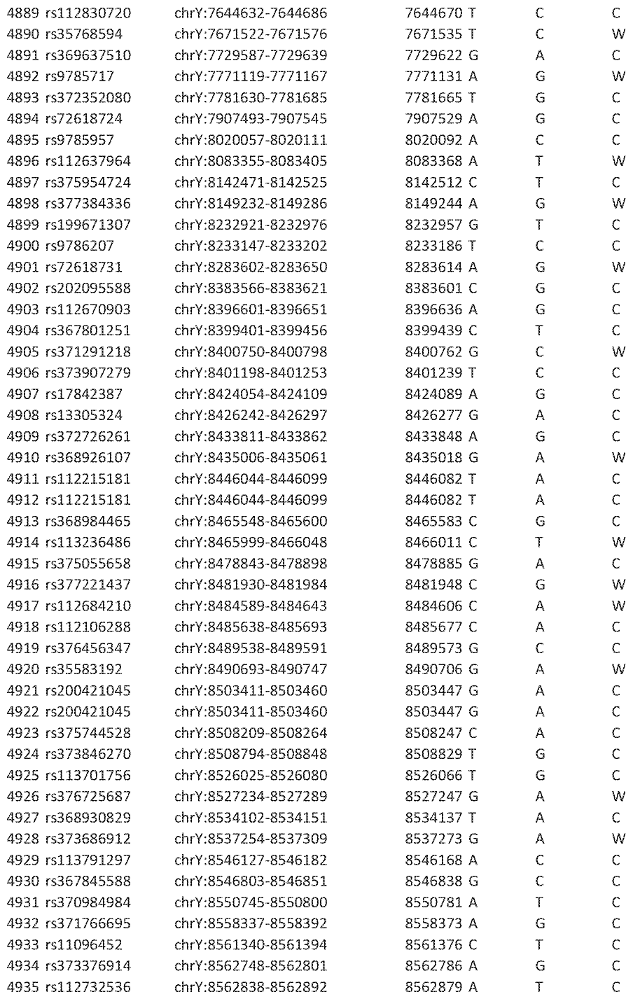

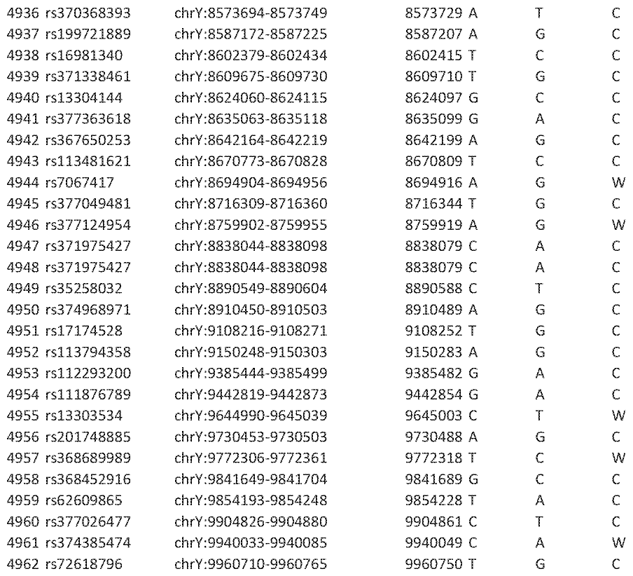

19. The plurality of polynucleotides of any one of embodiments 1-18, comprising between about 50 and about 150 polynucleotides (e.g., about 120 polynucleotides) configured to bind to a target sequence on human chromosome 1, e.g., any of target sequences 1-117 as set forth in Table 1 (the Table in FIG. 10), or a complementary or substantially complementary sequence thereof.

20. The plurality of polynucleotides of any one of embodiments 1-19, comprising between about 10 and about 50 polynucleotides (e.g., about 40 polynucleotides) configured to bind to a target sequence on human chromosome 2, e.g., any of target sequences 2747-2784 as set forth in Table 1 (the Table in FIG. 10), or a complementary or substantially complementary sequence thereof.

21. The plurality of polynucleotides of any one of embodiments 1-20, comprising between about 10 and about 80 polynucleotides (e.g., about 60 polynucleotides) configured to bind to a target sequence on human chromosome 3, e.g., any of target sequences 4072-4126 as set forth in Table 1 (the Table in FIG. 10), or a complementary or substantially complementary sequence thereof.

22. The plurality of polynucleotides of any one of embodiments 1-21, comprising between about 10 and about 80 polynucleotides (e.g., about 50 polynucleotides) configured to bind to a target sequence on human chromosome 4, e.g., any of target sequences 4127-4171 as set forth in Table 1 (the Table in FIG. 10), or a complementary or substantially complementary sequence thereof.

23. The plurality of polynucleotides of any one of embodiments 1-22, comprising between about 10 and about 80 polynucleotides (e.g., about 40 polynucleotides) configured to bind to a target sequence on human chromosome 9, e.g., any of target sequences 4172-4212 as set forth in Table 1 (the Table in FIG. 10), or a complementary or substantially complementary sequence thereof.

24. The plurality of polynucleotides of any one of embodiments 1-23, comprising between about 100 and about 1,500 polynucleotides (e.g., about 1,200 polynucleotides) configured to bind to a target sequence on human chromosome 13, e.g., any of target sequences 118-1337 as set forth in Table 1 (the Table in FIG. 10), or a complementary or substantially complementary sequence thereof.

25. The plurality of polynucleotides of any one of embodiments 1-24, comprising between about 10 and about 150 polynucleotides (e.g., about 100 polynucleotides) configured to bind to a target sequence on human chromosome 15, e.g., any of target sequences 1338-1444 as set forth in Table 1 (the Table in FIG. 10), or a complementary or substantially complementary sequence thereof.

26. The plurality of polynucleotides of any one of embodiments 1-25, comprising between about 100 and about 1,500 polynucleotides (e.g., about 1,200 polynucleotides) configured to bind to a target sequence on human chromosome 18, e.g., any of target sequences 1445-2681 as set forth in Table 1 (the Table in FIG. 10), or a complementary or substantially complementary sequence thereof.

27. The plurality of polynucleotides of any one of embodiments 1-26, comprising between about 10 and about 100 polynucleotides (e.g., about 60 polynucleotides) configured to bind to a target sequence on human chromosome 19, e.g., any of target sequences 2682-2746 as set forth in Table 1 (the Table in FIG. 10), or a complementary or substantially complementary sequence thereof.

28. The plurality of polynucleotides of any one of embodiments 1-27, comprising between about 100 and about 1,500 polynucleotides (e.g., about 1,200 polynucleotides) configured to bind to a target sequence human chromosome 21, e.g., any of target sequences 2785-3995 as set forth in Table 1 (the Table in FIG. 10), or a complementary or substantially complementary sequence thereof.

29. The plurality of polynucleotides of any one of embodiments 1-28, comprising between about 10 and about 120 polynucleotides (e.g., about 70 polynucleotides) configured to bind to a target sequence on human chromosome 22, e.g., any of target sequences 3996-4071 as set forth in Table 1 (the Table in FIG. 10), or a complementary or substantially complementary sequence thereof.

30. The plurality of polynucleotides of any one of embodiments 1-29, comprising between about 100 and about 500 polynucleotides (e.g., about 300 polynucleotides) configured to bind to a target sequence on human chromosome X, e.g., any of target sequences 4213-4462 as set forth in Table 1 (the Table in FIG. 10), or a complementary or substantially complementary sequence thereof.

31. The plurality of polynucleotides of any one of embodiments 1-30, comprising between about 300 and about 800 polynucleotides (e.g., about 500 polynucleotides) configured to bind to a target sequence on human chromosome Y, e.g., any of target sequences 4463-4962 as set forth in Table 1 (the Table in FIG. 10), or a complementary or substantially complementary sequence thereof.

32. The plurality of polynucleotides of any one of embodiments 1-31, comprising between about 300 and about 4,500 polynucleotides (e.g., about 3,600 polynucleotides) configured to bind to target sequences on human chromosomes 13, 18, and 21.

33. The plurality of polynucleotides of any one of embodiments 1-32, comprising between about 120 and about 800 polynucleotides (e.g., about 540 polynucleotides) configured to bind to target sequences on one or more human autosomes other than chromosomes 13, 18, and 21.

34. The plurality of polynucleotides of any one of embodiments 1-33, wherein the nucleic acid target comprises fragmented DNA of between about 100 and about 200 nucleotides in length (e.g., about 150 nucleotides in length).

35. A method for analyzing a fetal genetic information, e.g., fetal fraction, a chromosome abnormality such as trisomy, sex determination and/or prenatal paternity test, comprising:
a) contacting a sample from a female subject with the plurality of polynucleotides of any one of embodiments 1-34; and
wherein nucleic acid sequence information of the sample is obtained, which indicates a fetal genetic information.

36. The method of embodiment 35, wherein the female subject is known to be pregnant or suspected of being pregnant.

37. The method of embodiment 35 or 36, wherein the sample is a blood, serum, plasma, buccal swab, urine, saliva, tear, or body fluid sample.

38. The method of any one of embodiments 35-37, wherein the sample is freshly isolated or archived.

39. The method of any one of embodiments 35-38, wherein the sample comprises genomic DNA and/or cfDNA.

40. The method of any one of embodiments 35-39, wherein the sample comprises both maternal DNA and fetal DNA.

41. The method of any one of embodiments 35-40, wherein the sample is obtained by a method comprising a blood collection step, a sample transportation step, a plasma preparation step, and/or a cfDNA extraction step, before the contacting step.

42. The method of any one of embodiments 35-41, further comprising:
b) allowing the plurality of polynucleotides to bind to nucleic acid targets in the sample.

43. The method of embodiment 42, further comprising:
c) allowing the first and second target-specific domains of each polynucleotide bound to its nucleic acid target sequences to connect with each other such that the polynucleotide forms a circle.

44. The method of embodiment 43, wherein the connection is achieved by ligation.

45. The method of embodiment 43, wherein the connection is achieved by polymerase-mediated extension of the first or second target-specific domain, followed by ligation of the extended first (or second) target-specific domain to the second (or first) target-specific domain, or by ligation of the extended second (or first) target-specific domain to the first (or second) target-specific domain.

46. The method of any one of embodiments 43-45, further comprising:
d) eliminating polynucleotides that are not in circular form, e.g., polynucleotides that are not bound to any nucleic acid target and/or polynucleotides whose first and second target-specific domains are not connected in step c).

47. The method of embodiment 46, wherein the polynucleotides to be eliminated are linear, and step d) comprises contacting the sample from step c) with a nuclease, such as an exonuclease, e.g., Exo I and/or III.

48. The method of embodiment 46 or 47, further comprising:
e) releasing polynucleotides that are in circular form from their nucleic acid targets.

49. The method of embodiment 48, wherein the releasing comprises cleaving the linkers of the polynucleotides that are in circular form.

50. The method of embodiment 48 or 49, further comprising:
f) an enrichment step, such as an amplification reaction, of the released polynucleotides.

51. The method of embodiment 50, wherein the amplification reaction is a polymerase chain reaction (PCR), e.g., PCR using one or more primers in the linkers, a reverse-transcription PCR amplification, allele-specific PCR (ASPCR), single-base extension (SBE), allele specific primer extension (ASPE), strand displacement amplification (SDA), transcription mediated amplification (TMA), ligase chain reaction (LCR), nucleic acid sequence based amplification (NASBA), primer extension, rolling circle amplification (RCalif.), self-sustained sequence replication (3SR), the use of Q Beta replicase, nick translation, or loop-mediated isothermal amplification (LAMP), or any combination thereof.

52. The method of embodiment 50 or 51, further comprising:
g) obtaining the nucleic acid sequence information of the released polynucleotides, such as by hybridization-based detection and/or sequencing, including the observed UMI counts.

53. The method of embodiment 52, further comprising:
h) analyzing the nucleic acid sequence information obtained in step g).

54. The method of embodiment 53, which is configured for analyzing fetal fraction, a chromosome abnormality such as trisomy, sex determination and/or prenatal paternity test.

55. The method of embodiment 54, wherein the analyzing step comprises analyzing the fetal fractions and the UMI counts, and choosing the fetal fraction that best explains the observed UMI counts.

56. The method of embodiment 55, where in the analyzing step comprising assuming that the genotypes of the fetus and the female subject are known as g_{c} and gₘ, respectively, and calculating the frequencies of the polymorphic nucleotides (such as SNP nucleotides) according to the following formula:

$$p = \frac{{fg}_{c} + ( 1 - f ) {g}_{m}}{2}$$

where f is the fetal fraction, and g_{c} and gₘ, are the genotypes of the fetus and the female subject, respectively (0 for AA, 1, for Aa, and 2 for aa).

57. The method of embodiment 54, which is configured for analyzing trisomy using a hybrid of a depth-based and genotype-based approach.

58. The method of embodiment 54, which is configured for sex determination using an extension of the trisomy depth model to the sex chromosomes.

59. The method of any one of embodiments 54-58, which is conducted without using or referring to a known genotype.

60. The method of embodiment 54, which is configured for prenatal paternity test using the following analysis:
1) using a fully marginalized model to determine fetal fraction and produce a ‘baseline’ likelihood;
2) conducting a second analysis through the model to perform the same calculation as in step 1) using a putative father's genotype to constrain the genotypes to only those consistent with inheriting a paternal allele to obtain a second likelihood; and
3) deciding against paternity of the putative father when the ratio (P[data|baseline]/P[data|paternal] is more than a threshold, e.g., (P[data|baseline]/P[data|paternal] >10).

61. A kit for analyzing a fetal genetic information, e.g., fetal fraction, a chromosome abnormality such as trisomy, sex determination and/or prenatal paternity test, comprising a plurality of polynucleotides of any one of embodiments 1-34.

62. The kit of embodiment 61, which further comprises a reagent and/or a container for obtaining, preparing, isolating, enriching, purifying, storing and/or transporting a sample, e.g., a blood, serum, plasma, buccal swab, urine, saliva, tear, or body fluid sample.

63. The kit of embodiments 61 or 62, which further comprises a reagent for obtaining, preparing, isolating, enriching, purifying, storing and/or transporting polynucleotides, e.g., genomic DNA and/or cfDNA, from a sample.

64. The kit of any one of embodiments 61-63, wherein the polynucleotides comprise both maternal DNA and fetal DNA.

65. The kit of any one of embodiments 61-63, which further comprises a ligase.

66. The method of embodiment 65, which further comprises an enzyme, e.g., a polymerase, and/or another reagent for polymerase-mediated extension of the first or second target-specific domain.

67. The kit of any one of embodiments 61-66, which further comprises a reagent, e.g., an enzyme, a buffer or a washing solution, for eliminating polynucleotides that are not in circular form, e.g., polynucleotides that are not bound to any nucleic acid target and/or polynucleotides whose first and second target-specific domains are not connected.

68. The method of embodiment 67, wherein the enzyme is a nuclease, such as an exonuclease, e.g., Exo I and/or III.

69. The method of embodiment 67 or 68, which further comprises a reagent, e.g., an enzyme or a polymerase, for enriching or amplifying the released polynucleotides.

70. The kit of embodiment 69, wherein the reagent, e.g., an enzyme, is configured to be used in amplification reaction selected from the group consisting of a polymerase chain reaction (PCR), e.g., PCR using one or more primers in the linkers, a reverse-transcription PCR amplification, allele-specific PCR (ASPCR), single-base extension (SBE), allele specific primer extension (ASPE), strand displacement amplification (SDA), transcription mediated amplification (TMA), ligase chain reaction (LCR), nucleic acid sequence based amplification (NASBA), primer extension, rolling circle amplification (RCalif.), self-sustained sequence replication (3SR), the use of Q Beta replicase, nick translation, loop-mediated isothermal amplification (LAMP), and any combination thereof.

71. The kit of any one of embodiments 61-70, which further comprises a reagent, e.g., an enzyme, for obtaining the nucleic acid sequence information of the released polynucleotides, such as by hybridization-based detection and/or sequencing, including the observed UMI counts.

72. The kit of embodiment 71, which further comprises means for analyzing the nucleic acid sequence information.

73. The kit of embodiment 72, wherein the means is configured for analyzing fetal fraction, a chromosome abnormality such as trisomy, sex determination and/or prenatal paternity test.

The methods of the invention are illustrated by the following description and examples: based on these, the skilled person can apply the methods to a variety of samples and targets. The examples are illustrative and are not to be seen as limiting the scope of the invention.

A plurality of single stranded DNA connector inversion probes are typically used to simultaneously capture selected human genomic DNA regions which contain one or more SNPs on chromosome 21, 18, 13, X, Y, and some other autosomal chromosomes. The captured regions provide both SNP information and depth coverage information which allow the user to simultaneously measure fetal fraction and better characterize Trisomy, for example.

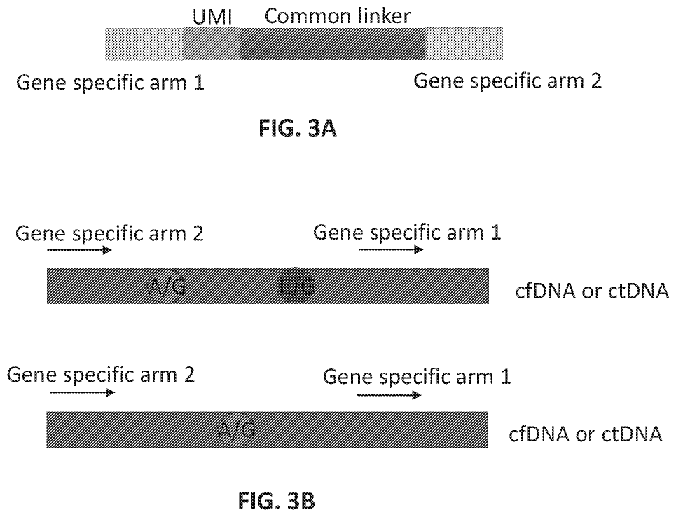

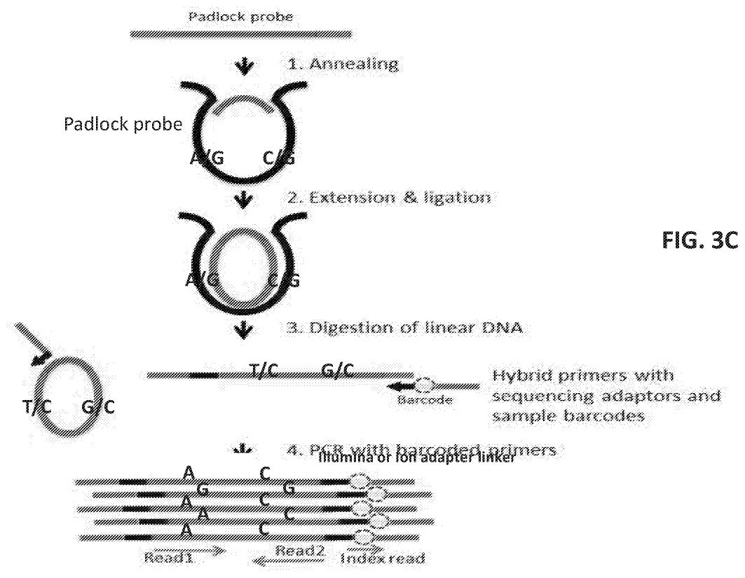

Target selection and connector inversion probes design are illustrated in FIG. 3. Human genomic DNA regions containing one or more SNPs are selected as potential targets. The SNPs are selected based on the level of heterozygosity in the major populations. Single stranded DNA connector inversion probes are used to capture selected regions of human genomic DNA. Connector inversion probes, or ‘padlock probes,’ can be designed using software described in published US patent application 20140357497 A1 patent by methods previously described [21.22]. A Single stranded DNA connector inversion probe is typically between 90 to 100 nucleotides in length, and can be structured as shown in FIG. 3A. From 5′ end to 3′ end, the probe may comprise around 20 nucleotides in gene specific arm 1; 6 to 8 nucleotides in a unique molecule identifier (UMI), around 50 nucleotides of a common linker; and around 20 nucleotides in gene specific arm 2. Gene specific arm 2 and arm 1 are chosen so they will anneal to the complementary region on human genomic DNA in the target region. The gap between 3′ end of Arm 2 and 5′ end of Arm 1 can be between 1 nucleotide to 100 nucleotides, and preferably contains at least one SNP (For example, A or G) as shown in FIG. 3B. Connector inversion probes precursors can be synthesized by CustomArray Inc. (Bothell, Wash., USA). The working probes are produced through PCR amplification, DpnII/User double digestions and then recovered as single stranded DNA probes from 6% denatured Urea PAGE. Alternatively, the working connector inversion probes can be directly synthesized by IDT (Coralville, Iowa, USA).

Capture method and optimization. The procedures to capture the human genomic targets using single stranded connector inversion probes were described previously [21,22]. As illustrated in FIG. 3C, fragmented genomic DNA fragments or cfDNA are incubated with single stranded connector inversion probes at a proper ratio for 20 to 24 hours at a suitable temperature, e.g., 58° C. A mixture of enzymes containing heat stable DNA polymerase and DNA ligase is added for 1 hour at 58° C. to fill the gap between the two gene specific arms. ExoI/III mixture is added next to digest unannealed single stranded probes. The captured targets are further enriched and indexed for the individual sample by PCR with either Illumina adapter or Ion Torrent adapter. DNA libraries are quantified by qPCR. Alternatively, Roiling Cycle Amplification (RCalif.) can be used to amplify the captured target. The capture condition of Afisawa are optimized to capture either short fragmented genomic DNA or cfDNA. Either high through put DNA sequencing or Array could be used to detect the signals.

Input DNA. Genomic DNA can be fragmented with ˜150 bp peak using Corvaris, purified using AmpureXP and the concentration measured by Qubit. DNA from mother and DNA from son or daughter can be mixed at proper ratio to create a DNA mixture to mimic cfDNA from pregnant women with various fetal fraction and/or T21 pregnancy. cfDNA can be extracted from the plasma of pregnant women by methods well-known in the art.

Connector inversion probes selection. Around 5000 probes were selected as final working probes for Afisawa assay from initial 14,000 probes designed using licensed software (in US 20140357497 A1 patent-Designing padlock probes for targeted genomic sequencing (Kun Zhang, Athurva Gore)) as previously described [21,22].

### Example 1

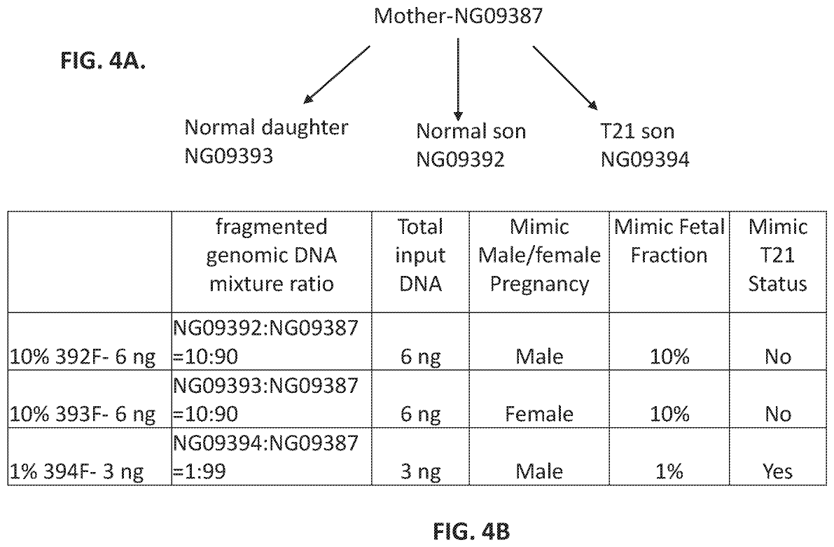

Using the methods described above, reference samples and test samples were subjected to Afisawa testing, and the results are shown in FIGS. 4-6. Illumina libraries were sequenced using customized sequencing primer for read 1 of 150 bp and index 1 of 6 bp and sequenced on either Miseq, Miniseq, or Nextseq500. Ion Torrent libraries were generated using Ion express barcodes and sequenced on Ion S5 or Ion Proton. Genomic DNAs were purchased from Corriell Institute. Genomic DNA was fragmented with ˜150 bp peak using Corvaris, purified using AmpureXP and the concentration was measured by Qubit. Fragmented genomic DNA from mother and DNA from son or daughter were mixed at proper ratio to create a DNA mixture to mimic cfDNA from pregnant women with various fetal fraction and/or T21 pregnancy.

As depicted in FIG. 4A, samples were prepared to mimic several combinations of gender and T21 status, and were prepared to include fetal DNA mixed with maternal DNA in typical proportions for samples from maternal plasma (FIG. 4B). Afisawa analysis was conducted with appropriate probes to analyze gender, fetal DNA fraction, and T21 status. Fastq files from Illumina sequencer were subjected to Afisawa data analysis. For data from Lifetech S5, Bam file was converted to fastq file first.

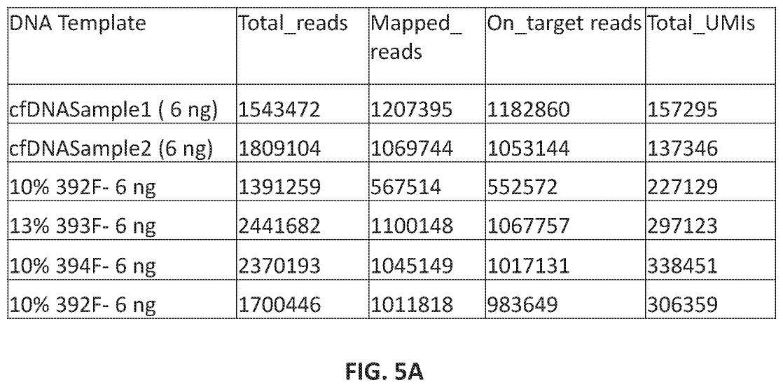

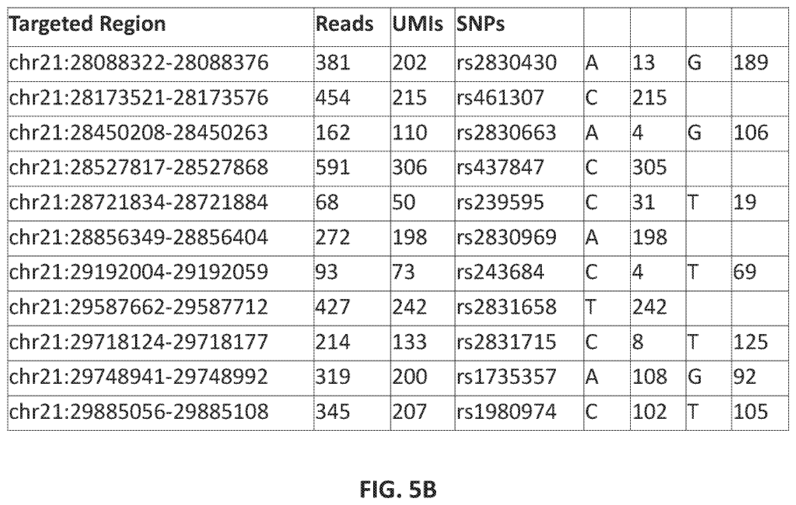

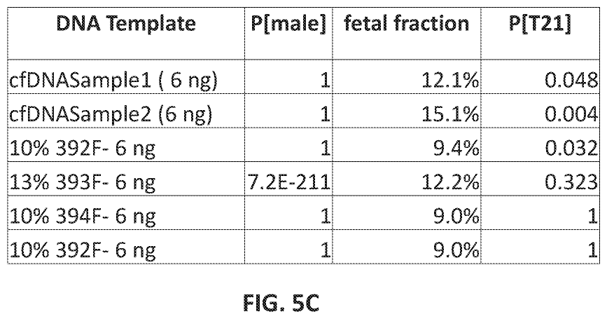

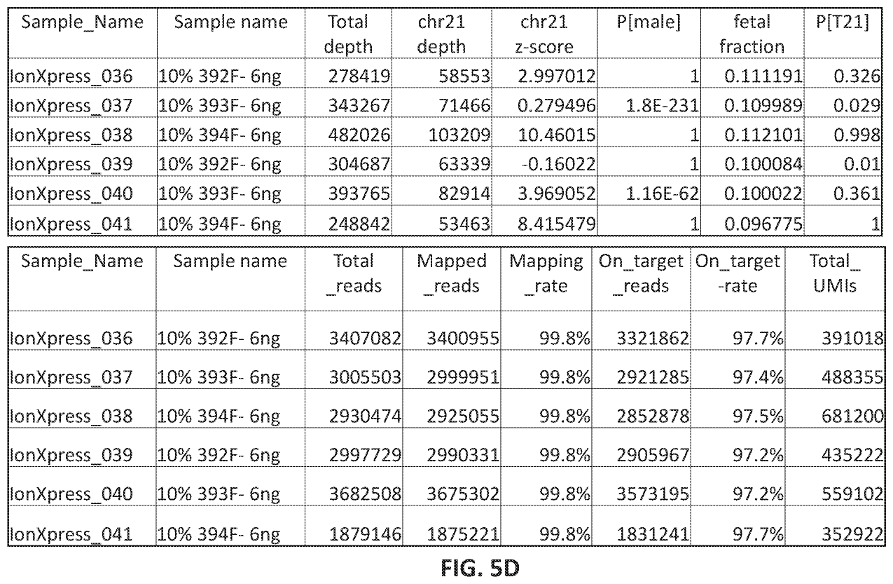

6 ng either cfDNA from a pregnant woman or genomic DNA mixture was used as input for Afisawa assay. Fastq file from each sample went through the mapping pipeline. Total-UMIs represent the total number of targets being captured (FIG. 5B). For each selected target, there are in total 381 reads that passed quality control and were mapped to the target. 202 UMIs were observed. 189 targets contain SNP of G and 13 targets contain SNP of A. 202 UMIs were used to calculate Z score based on coverage depth. The ratio of SNP of A to SNP of G was used to calculate the fetal fraction. Probability of Trisomy was calculated based on coverage depth and SNPs information). Sample output of Afisawa is reported from Illumina libraries being sequenced on Miseq (FIG. 5C) and from Ion libraries being sequenced on S5 (FIG. 5D).

The human mapped reads, on targeted reads, total UMIs, the number of UMI containing different SNP are shown in FIG. 5A and 5B. FIGS. 5C and 5D show the final report of Afisawa from Illumina libraries or Ion libraries, respectively, for the samples from FIG. 4.

### Fetal Fraction Estimate

The fetal fraction estimate is produced by choosing the fraction of fetal reads which best explains the observed UMI counts. The basis for the model is the observation that SNP allele counts are informative whenever the fetal genotype differs from the maternal genotype. While neither of these genotypes are known, we observe that (i) the parents are unrelated and (ii) each SNP has a known population frequency. These two observations enable one to propagate allele count information through the genotype uncertainty.

Specifically: assuming the genotypes are known, the SNP allele counts follow a standard binomial distribution, with frequencies of

$$p = \frac{{fg}_{c} + ( 1 - f ) {g}_{m}}{2}$$

where f is the fetal fraction, and g_{c} and gₘ are the genotypes of the child and mother, respectively (0 for AA, 1, for Aa, and 2 for aa).

The count-model is nested within the genotype model. gm is a binomial draw from the population frequency, q; while g_{c} must share one allele with gₘ due to Mendelian inheritance, and the other allele is randomly drawn from the population with frequency q. We can then use Bayes' rule to maximize:

$$P [ f \vert D ] = P [ D \vert f ] = {\sum }_{{g}_{c} , {g}_{m}} P [ D \vert f , {g}_{c} , {g}_{m} ] P [ {g}_{c} \vert {g}_{m} ] P [ {g}_{m} ]$$

### Trisomy Model

The trisomy model is a hybrid of a depth-based and genotype-based approach. For the depth model, by knowing the number of probes for each chromosome, we can calculate the expected number of UMI for each chromosome under normal, trisomy, and haploid states. In particular:

$${N}_{21}^{( dip )} = N \frac{2 * {M}_{21}}{2 * ( {M}_{1} + {M}_{2} + ⋯ + {M}_{22} + {M}_{ϰ} )} = {Nq}_{21}^{( dip )}$$

$${N}_{21}^{( trip )} = N \frac{3 * {M}_{21}}{2 * ( {M}_{1} + {M}_{2} + ⋯ + {M}_{20} + {M}_{22} + {M}_{ϰ} ) + 3 * {M}_{21}} = {Nq}_{21}^{( trip )}$$

where N is the total number of UMI, and Mᵢ is the number of probes on chromosome i. The variances of these estimates are Nq(1−q); and they are approximately normal due to the law of large numbers. Then at a fetal fraction f, the observed number of UMI in the triploid state follows a normal distribution with mean

N₂₁^{(obs,trip)}=fN₂₁^{(trip)}+(1−f)N₂₁^{(dip)}

and variance fNq₂₁^{(trip)(}1−q₂₁ ^{(trip)})+(1−f)Nq₂₁^{(dip)}(1−q₂₁^{(dip)}). We summarize the observed UMI as both a Z-score under the pure N₂₁^{(dip)} distribution, as well as a Bayes factor for N₂₁^{(obs,trip)} vs N₂₁^{(dip)}.

The genotype-based trisomy model follows the same approach as the fetal fraction model, but instead contrasts a trisomy or a normal model. Briefly

$$P [ S \vert D ] = P [ D \vert S ] = {\sum }_{{g}_{c} , {g}_{m}} P [ D \vert {g}_{c} , {g}_{m} ] P [ {g}_{c} \vert {g}_{m} , S ] P [ {g}_{m} ] P [ S ]$$

where S is either ‘trisomy’ or ‘normal’.

The observed UMI counts are again binomial, with the mean and variance given by the (fetal fraction)-weighted average of the child and mother allele frequencies. The likelihood child's genotype state is dependent on two additional unknown factors: i) which parent contributed the extra chromosome, and ii) which meiotic division resulted in the duplication. For instance, a paternal first-division nondisjunction will contribute both of the father's alleles; while a paternal second-division nondisjunction will contribute one of the father's alleles at copy number 2. Based on epidemiological studies, the probability of paternal origin is set at 8.3%, and the probability of first-division nondisjunction is set at 30%. The evidence of trisomy based on SNP allele counts is summarized as a Bayes factor for P[trisomy|D] vs P[norma|D].

The final trisomy adding the depth and genotype Bayes factors, with larger scores corresponding to higher confidence in the presence of an extra copy of chromosome 21.

### Sex Determination

Applying this method for sex determination is an extension of the trisomy depth model to the sex chromosomes. In particular

$${q}_{y}^{( male )} = \frac{{M}_{Y}}{2 * ( \text{?} + \text{?} + ⋯ \text{?} ) + {M}_{X} + {M}_{Y}}$$

$${q}_{y}^{( female )} = {10}^{- 6}$$

$${q}_{ϰ}^{( male )} - \frac{{M}_{X}}{2 * ( \text{?} + \text{?} + ⋯ + \text{?} ) + {M}_{X} + {M}_{Y}}$$

$${q}_{ϰ}^{( female )} = \frac{2 * {M}_{X}}{2 * ( \text{?} + \text{?} + ⋯ + \text{?} + {M}_{X} )}$$

$$\text{?} \text{indicates text missing or illegible when filed}$$

We model the observed X and Y counts as binomial distributions with rates

fqₓ^{(H)}+(1−f)qₓ^{(female)}; fqₓ^{(H)}+(1−f)q_{y}^{(female)}

respectively, with H the hypothesized child sex. Whichever hypothesis maximizes the posterior likelihood (after marginalizing) is selected as the observed sex.

### Prenatal Paternity Test

Afisawa can be potentially used as a prenatal paternity test if gene typing of the potential father's genomic DNA is available. The genotype model for fetal fraction is used; and the fully marginalized model is used to determine fetal fraction and produce a ‘baseline’ likelihood. A second pass through the model performs the same calculation, but instead of summing over all possible child genotypes, the putative father's genotypes are used to constrain the genotypes to only those consistent with inheriting a paternal allele. This results in a second likelihood. If this likelihood is ten times less likely than the baseline (P[data|baseline]/P[data|paternal]>10), then this is taken as evidence against paternity.

### Example 2

### Gender Identification

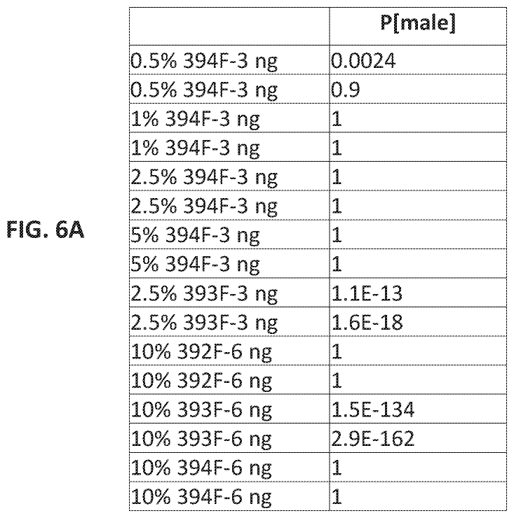

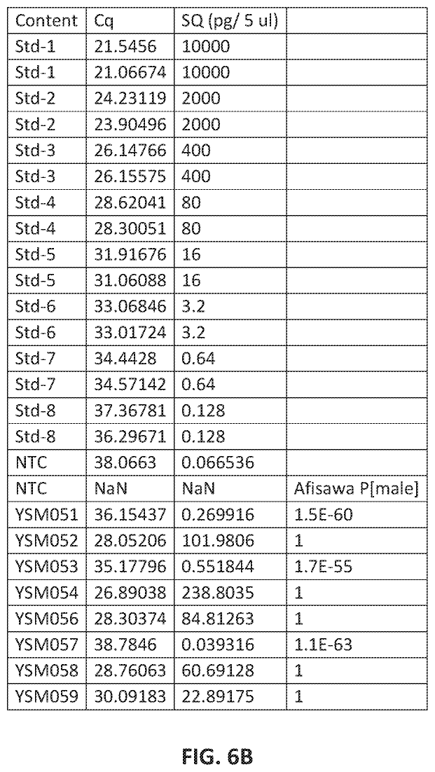

In this example, 200-500 single stranded connector inversion probes targeting human Y Chromosome were used to detect the male/female pregnancy. In the example of FIG. 6A, 3 ng or 6 ng fragmented genomic DNA mixture was used as input for Afisawa assay. As low as 10 copies of male gDNA (30 pg, 1% NG09394F of 3 ng total input) can be detected by Afisawa. P[male] of 0.95 or higher is considered as male pregnancy. When cfDNA from pregnant women was used as input in the example of FIG. 6B, all male/female pregnancy were correctly identified. 2 ng cfDNA isolated from plasma of a pregnant woman was used for qPCR using syber green based DYZS. qPCR assay using fragmented male genomic DNA as standards, 1 pg or more was called as male. Male/female detection by Afisawa is highly correlated with DYZS qPCR results. Sex is determined on the basis of a y-chromosomal-based probability. Briefly, the number of reads expected on chrY from a female child is very small (e.g. the mismapping or contamination rates), while the number of reads expected on chrY from a male child is several orders of magnitude larger. Our estimates place the average mapping rate for males at ˜7e-5 for males and ˜5e-7 for females. On this basis we compute the binomial mapping probability (P[k UMI from y|N total umi]=(N choose k)×(q)^{k}(1−q)^{N-k} with q the sex-based mapping rate—see q_{y}^{(male)} and q_{y}^{(female)} above), and normalize the two scenarios (male, female) to have total probability 1.

### Example 3

### Fetal Fraction

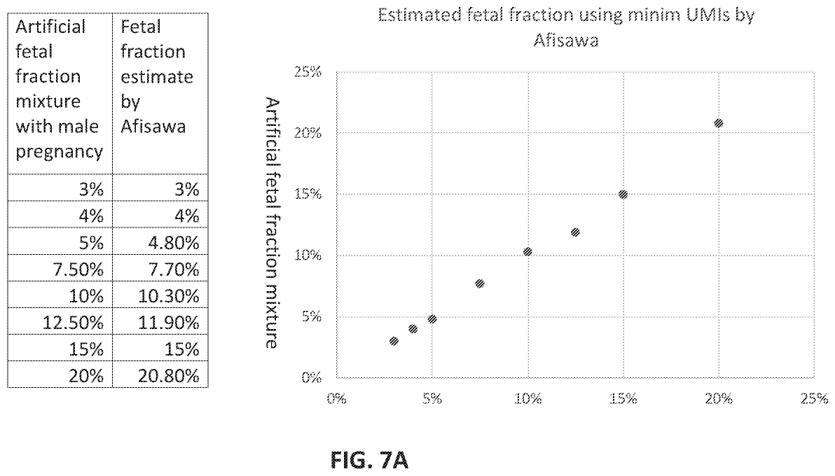

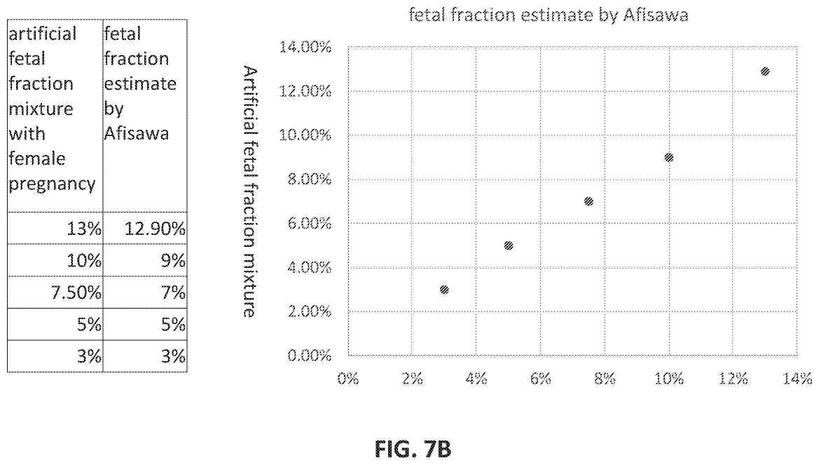

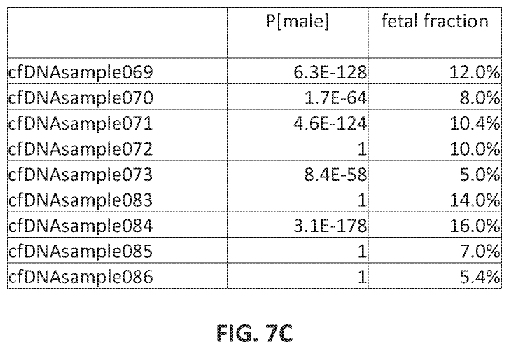

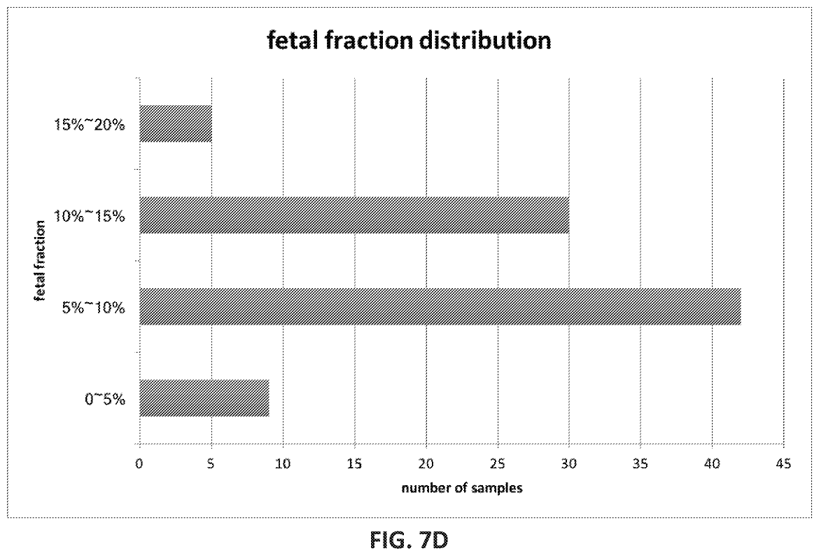

One major feature of Afisawa assay is its capability to measure the fetal fraction regardless of male or female pregnancy. Fragmented DNA mixtures mimicking either female pregnancy (FIG. 7A) or male pregnancy (FIG. 7B) were used as input for Afisawa assay, as low as 3% fetal fraction can be measured for both female and male pregnancy.

FIG. 7B. Fragmented DNA from mother (NG09387) and DNA from son (NG09394) were mixed at proper ratio to create an artificial fetal fraction DNA mixture to mimic cfDNA from pregnant women with male pregnancy. Percentage of artificial fetal fraction mixtures with male pregnancy is plotted with respect to the fetal fraction estimated by Afisawa. FIG. 7C: 6 ng cfDNA from pregnant woman was used as input for Afisawa assay. Afisawa can measure fetal fraction of cfDNA from pregnant women with both male and female pregnancy. FIG. 7D: Fetal fraction distribution from 86 cfDNA isolated from plasma of pregnant women. The average fetal fraction is ˜9%;

In the example of FIG. 7C, fetal fraction of cfDNA from pregnant women was measured using Afisawa assay. FIG. 7D showed the fetal fraction distribution from 64 cfDNA isolated from plasma of pregnant women. The average fetal fraction is ˜9%. Fetal fractions are estimated on the basis of a Bayesian model. Given a known fetal fraction and known fetal genotype, the probability of observing k reference alleles and N−k alternate alleles is) Σᵢ₌₁^{k}(1−f)P[uⱼ|g_{M}]+fP[uᵢ|g_{c}] where uᵢ is consensus base of the i^{th} UMI, and g_{M} and g_{C} are the mother and child UMI respectively. These probabilities are straightforward genotyping; for homozygous genotypes (g*=AA): P[A|AA]=(1−pₑ); P[B|AA]=pₑ; where pₑ is the error rate quantified by the consensus base quality score. For heterozygous genotypes (g*=AB): P[A|AB]=P[B|AB]=0.5*(1−pₑ)+0.5*pₑ. In this way, a given combination of (fetal fraction, genotype) can be scored for every marker used in the assay. Fetal fraction estimation proceeds by marginalization; that is at each marker we sum over all maternal and child genotypes, producing one likelihood per marker. By doing this a given fetal fraction can be scored at each marker. Combining these likelihoods by summing them across all markers, produces a single score for a given fetal fraction. Finally, for each value of fetal fraction from 0.005 to 0.20 (in increments of 0.005) one can compute that likelihood, producing an array of 40 likelihoods; which is then normalized to have total probability 1. The final fetal fraction estimate is the mean of the resulting distribution, i.e. Σ_{f=i}^{4.0} 0.005*f*P[0.005*f].

### Example 4

### Trisomy Call

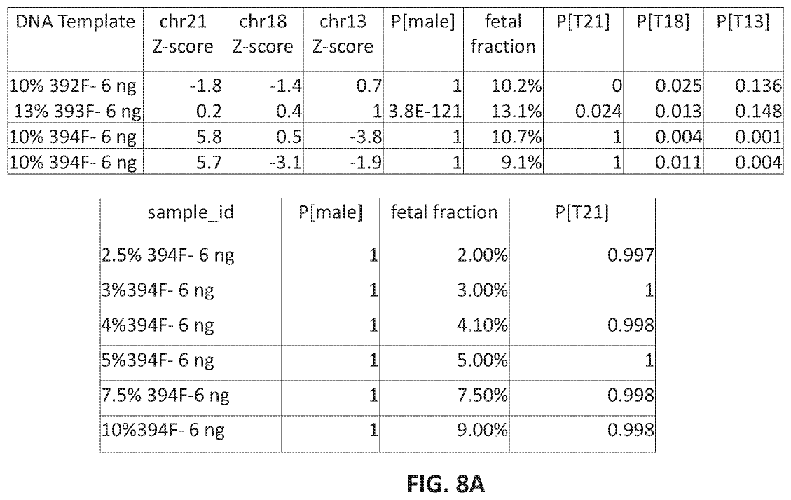

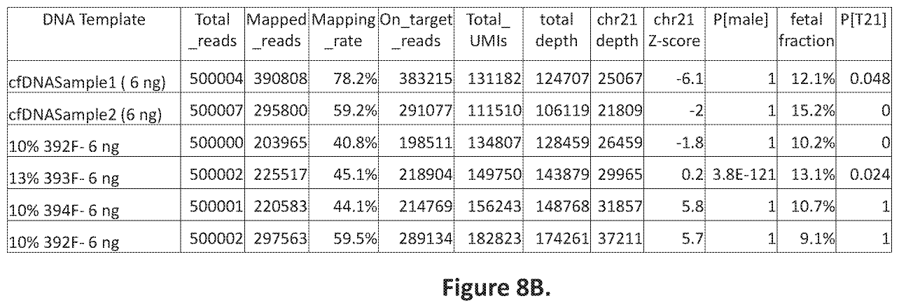

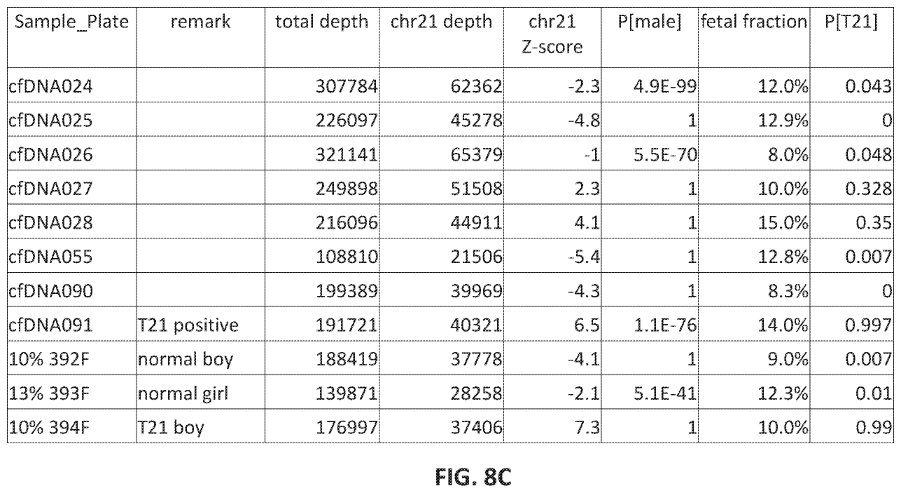

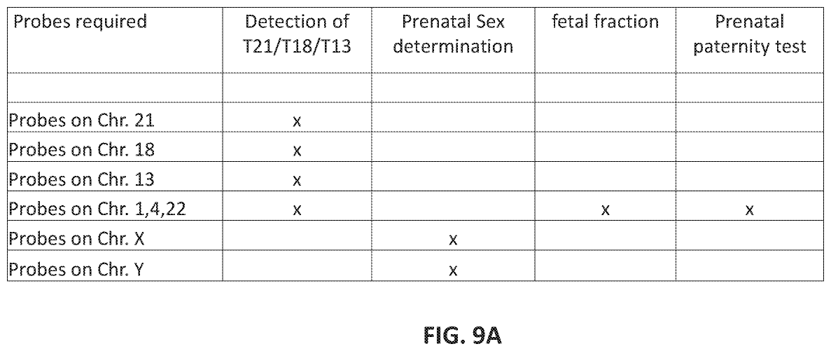

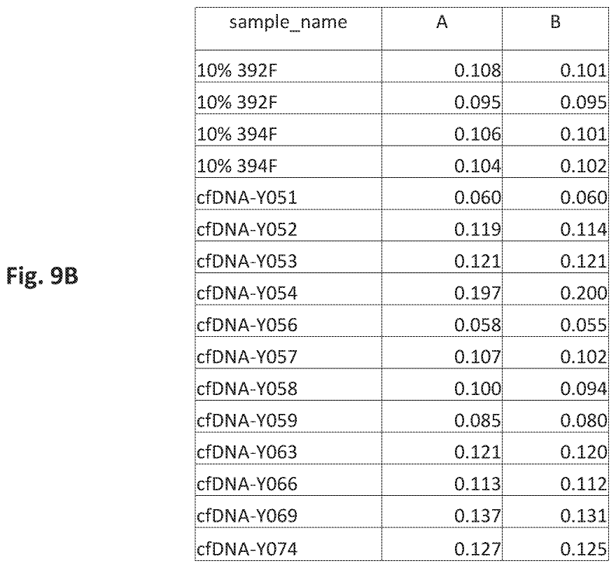

In the example summarized in FIG. 8A, fragmented genomic DNA mixture mimicking cfDNA from T21 pregnancy or normal pregnancy was used for Afisawa NIPT assay. T21 could be detected for the genomic DNA mixture with as low as 3% artificial fetal fraction. In the example of FIG. 8B, only a half million raw reads are sufficient for P[male], fetal fraction estimate, and P[T21] classification using Afisawa, thus a desktop high through put DNA sequencer such as Illumina Miseq, Miniseq or Lifetech Ion torrent, S5 is sufficient for the Afisawa assay. In the example of FIG. 8C, cfDNA isolated from plasma of pregnant women or a mixture of reference genomic DNA fragments was subjected to Afisawa NIPT assay or NIPT (MPSS). Afisawa can detect T21 from cfDNA of pregnant women-cfDNA091. Trisomy classification is performed on the basis of read depth. Our assay has an expected mapping rate for each chromosome, which (while empirically calculated) is based on the total number of probes on each chromosome and the efficiency of those probes. Given this mapping rate for each chromosome (in terms of a normal female), the expected mapping rate can be computed for any karyotype, including male and trisomy samples using the formulas presented in (8). From this, expected mapping rates are produced for mother/normal mix, and mother/trisomy mix at the estimated fetal fraction. The variance of these rates is determined by the total number of UMI using the multinomial distribution; in particular if the mapping rate is r, and there are N UMI, then the variance is r(1−r)/N. Since both states have a mean and a variance, two Z-scores can be produced (Zₙ and Zₜ). These can be converted into likelihoods via e^{-Z\hat{ }2/2} and normalized to have total probability one. The resulting probabilities are Pₙ and Pₜ; and a threshold of Pₜ>0.9 is taken as a threshold for a T21 call; while Pₜ<0.2 is taken as a threshold for a normal call. ‘Indeterminate’ is assigned to samples with 0.2<Pₜ<0.9.

One to four applications (fetal fraction estimate, fetal sex determination, Trisomy call, prenatal paternity test) of Afisawa can be achieved by utilizing the different combinations of probes on target capture and/or data analysis from a single test.

Based on the foregoing, the skilled person can design suitable polynucleotides to use as probes for the methods of the invention. The probes can be directed to many different targets of interest for NIPT testing. The Table in FIG. 10 identifies nearly 5000 such targets that are of special interest for use in the methods of the invention. In the Table, each row of data begins with a row number for convenient reference. After the column of row numbers, the subsequent columns are: Column A-Rsid (Reference SNP cluster ID); Column B-Chr. Location; Column C-SNP position; Column D-Ref (Reference sequence); Column E-Alt (alternate sequence); Column F-Watson/Crick. The rows in the Table are grouped according to the target as follows: Chr. 1 (rows 1-117); Chr. 13 (rows 118-1337); Chr. 15 (rows 1338-1444); Chr. 18 (rows 1445-2681); Chr. 19 (rows 2682-2746); Chr. 2 (rows 2747-2784); Chr. 21 (rows 2785-3995); Chr. 22 (rows 3996-4071); Chr. 3 (rows 4072-4126); Chr. 4 (rows 4127-4171); Chr. 9 (rows 4172-4212); Chr. X (rows 4213-4462); Chr. Y (rows 4463-4962).

### REFERENCS

1. Gao Y, Xie B, Liu R. Delivering noninvasive prenatal testing in a clinical setting using semiconductor sequencing platform. Sci China Life Sci. 2014 July; 57(7):737-8. doi: 10.1007/s11427-014-4696-0. Epub 2014 Jun 26. No abstract available. PMID:24969704
2. Lo Y M, Corbetta N, Chamberlain P F, Rai V, Sargent I L, Redman C W, Wainscoat J S. Presence of fetal DNA in maternal plasma and serum. Lancet. 1997 Aug. 16; 350(9076):4857.PMID:9274585
3. Wong F C, Lo Y M Prenatal Diagnosis Innovation: Genome Sequencing of Maternal Plasma. Annu Rev Med. 2016; 67:419-32. doi: 10.1146/annurev-med-091014-115715. Epub 2015 Oct. 15.
4. Chiu R W, Chan K C, Gao Y, Lau V Y, Zheng W, Leung T Y, Foo C H, Xie B, Tsui N B, Lun F M, Zee B C, Lau T K, Cantor C R, Lo Y M. Noninvasive prenatal diagnosis of fetal chromosomal aneuploidy by massively parallel genomic sequencing of DNA in maternal plasma. Proc Natl Acad Sci U S A. 2008 Dec. 23; 105(51):20458-63. doi: 10.1073/pnas.0810641105. Epub 2008 Dec. 10.
5. Noninvasive diagnosis of fetal aneuploidy by shotgun sequencing DNA from maternal blood. Fan H C, Blumenfeld Y J, Chitkara U, Hudgins L, Quake S R. Proc Natl Acad Sci U S A. 2008 Oct. 21; 105(42):16266-71. doi: 10.1073/pnas.0808319105. Epub 2008 Oct. 6. PMID:18838674
6. Non-invasive prenatal assessment of trisomy 21 by multiplexed maternal plasma DNA sequencing: large scale validity study. Chiu R W, Akolekar R, Zheng Y W, Leung T Y, Sun H, Chan K C, Lun F M, Go A T, Lau E T, To W W, Leung W C, Tang R Y, Au-Yeung S K, Lam H, Kung Y Y, Zhang X, van Vugt J M, Minekawa R, Tang MH, Wang J, Oudejans C B, Lau T K, Nicolaides K H, Lo Y M. BMJ. 2011 Jan. 11; 342:c7401. doi: 10.1136/bmj.c7401
7. DNA sequencing versus standard prenatal aneuploidy screening. Bianchi D W, Parker R L, Wentworth J, Madankumar R, Saffer C, Das AF, Craig J A, Chudova D I, Devers P L, Jones K W, Oliver K, Rava R P, Sehnert A J; CARE Study Group.N Engl J Med. 2014 Feb. 27; 370(9):799-808. doi: 10.1056/NEJMoa1311037
8. Cell-free DNA analysis for noninvasive examination of trisomy. Norton M E, Jacobsson B, Swamy G K, Laurent L C, Ranzini A C, Brar H, Tomlinson M W, Pereira L, Spitz J L, Hollemon D, Cuckle H, Musci T J, Wapner R J. N Engl J Med. 2015 Apr. 23; 372(17):1589-97. doi: 10.1056/NEJMoa1407349. Epub 2015 Apr. 1.
9. Non-Invasive Chromosomal Evaluation (NICE) Study: results of a multicenter prospective cohort study for detection of fetal trisomy 21 and trisomy 18.Norton M E, Brar H, Weiss J, Karimi A, Laurent L C, Caughey A B, Rodriguez M H, Williams J 3rd, Mitchell M E, Adair C D, Lee H, Jacobsson B, Tomlinson M W, Oepkes D, Hollemon D, Sparks A B, Oliphant A, Song K. Am J Obstet Gynecol. 2012 August; 207(2):137.e1-8. doi: 10.1016/j.ajog.2012.05.021. Epub 2012 Jun. 1.
10. Noninvasive prenatal detection and selective analysis of cell-free DNA obtained from maternal blood: evaluation for trisomy 21 and trisomy 18. Sparks A B, Struble C A, Wang E T, Song K, Oliphant A. Am J Obstet Gynecol. 2012 April; 206(4):319.e1-9. doi: 10.1016/j.ajog.2012.01.030. Epub 2012 Jan. 26. PMID:22464072
11. Selective analysis of cell-free DNA in maternal blood for evaluation of fetal trisomy. Sparks A B, Wang E T, Struble C A, Barrett W, Stokowski R, McBride C, Zahn J, Lee K, Shen N, Doshi J, Sun M, Garrison J, Sandler J, Hollemon D, Pattee P, Tomita-Mitchell A, Mitchell M, Stuelpnagel J, Song K, Oliphant A. Prenat Diagn. 2012 January; 32(1):3-9. doi: 10.1002/pd.2922. Epub 2012 Jan. 6.
12. Noninvasive prenatal aneuploidy testing of chromosomes 13, 18, 21, X, and Y, using targeted sequencing of polymorphic loci Zimmermann B, Hill M, Gemelos G, Demko Z, Banjevic M, Baner J, Ryan A, Sigurjonsson S, Chopra N, Dodd M, Levy B, Rabinowitz M. Prenat Diagn. 2012 December; 32(13):1233-41. doi: 10.1002/pd.3993. Epub 2012 Oct 30.
13. Agarwal, A., Sayres, L. C., Cho, M. K., Cook-Deegan, R., & Chandrasekharan, S. (2013). Commercial Landscape of noninvasive prenatal testing in the United States. Prenatal Diagnosis, 33(6), 521-531. http://doi.org/10.1002/pd.4101
14. The impact of maternal plasma DNA fetal fraction on next generation sequencing tests for common fetal aneuploidies. Canick J A, Palomaki G E, Kloza E M, Lambert-Messerlian G M, Haddow J E. Prenat Diagn. 2013 July; 33(7):667-74. doi: 10.1002/pd.4126. Epub 2013 May 31. PMID:23592541
15. Gregg A R et al, Noninvasive prenatal screening for fetal aneuploidy, 2016 update: a position statement of the American Colleg of Medical Genetics and Genomics, 2016, Genetics in Medicine.
16. Bioinformatics Approaches for Fetal DNA Fraction Estimation in Noninvasive Prenatal Testing. Peng X L, Jiang P.IntJMolSci.2017Feb. 20; 18(2).pii:E453.doi:10.3390/ijms18020453.Review.PMID:2823 0760
17. Nilsson M, Malmgren H, Samiotaki M, Kwiatkowski M, Chowdhary B P, Landegren U (1994). “Padlock probes: circularizing oligonucleotides for localized DNA detection”. Science. 265 (5181): 2085-208
18. Michael S. Akhras, Magnus Unemo, Sreedevi Thiyagarajan, Pål Nyrén, Ronald W. Davis, Andrew Z. Fire, and Nader Pourmand Connector Inversion Probe Technology: A Powerful One-Primer Multiplex DNA Amplification System for Numerous Scientific Applications PLoS One. 2007; 2(9): e915. Published online 2007 Sep. 19. doi: 10.1371/journal.pone.0000915
19. Porreca G J, Zhang K, Li JB, Xie B, Austin D, Vassallo S L, LeProust E M, Peck B J, Emig C J, Dahl F, Gao Y, Church G M, Shendure J (2007). “Multiplex amplification of large sets of human exons”. Nat Methods. 4 (11): 931-936.Multiplex padlock targeted sequencing reveals human hypermutable CpG variations.
20. Li J B, Gao Y, Aach J, Zhang K, Kryukov G V, Xie B, Ahlford A, Yoon J K, Rosenbaum A M, Zaranek A W, LeProust E, Sunyaev S R, Church G M. Genome Res. 2009 September; 19(9):1606-15. doi: 10.1101/gr.092213.109. Epub 2009 Jun. 12
21. Deng J, Shoemaker R, Xie B, Gore A, LeProust E M, Antosiewicz-Bourget J, Egli D, Maherali N, Park I H, Yu J, Daley G Q, Eggan K, Hochedlinger K, Thomson J, Wang W, Gao Y, Zhang K. Targeted bisulfite sequencing reveals changes in DNA methylation associated with nuclear reprogramming Nat Biotechnol. 2009 April; 27(4):353-60. doi: 10.1038/nbt.1530. Epub 2009 Mar. 29.
22. Library-free methylation sequencing with bisulfite padlock probes. Diep D, Plongthongkum N, Gore A, Fung HL, Shoemaker R, Zhang K. Nat Methods. 2012 Feb. 5; 9(3):270-2. doi: 10.1038/nmeth.1871.

1. A plurality of polynucleotides, wherein each polynucleotide comprises:
a first target-specific domain and a second target-specific domain configured to bind to a first target sequence and a second target sequence, respectively, of a nucleic acid target, and
a unique molecule identifier (UMI) and a linker between the first and second target-specific domains,
wherein the first and second target-specific domains are configured to be connected to each other such that the polynucleotide forms a circle, optionally after a polymerase-mediated extension of the first or second target-specific domain, and
wherein the nucleic acid target comprises a polymorphic nucleotide within the first target sequence and/or the second target sequence, or between the first and second target sequences.

2. The plurality of polynucleotides of claim 1, which are single-stranded polynucleotides.

3. (canceled)

4. The plurality of polynucleotides of claim 1, which are between about 50 nucleotides and about 200 nucleotides in length, e.g., between about 90 nucleotides and about 100 nucleotides in length.

5-7. (canceled)

8. The plurality of polynucleotides of claim 1, wherein the linker comprises one or more common nucleotides for subsequent PCR annealing.

9-11. (canceled)

12. The plurality of polynucleotides of claim 1, wherein the nucleic acid target is from a sex chromosome, such as a chromosome X or chromosome Y, or from an autosome.

13. The plurality of polynucleotides of claim 1, wherein the nucleic acid target is from a mammalian chromosome, such as a human chromosome.

14. The plurality of polynucleotides of claim 1, wherein the plurality of polynucleotides are configured to bind to a target sequence on human chromosome 1, human chromosome 2, human chromosome 3, human chromosome 4, human chromosome 9, human chromosome 13, human chromosome 15, human chromosome 18, human chromosome 19, human chromosome 21, human chromosome 22, human chromosome X, or human chromosome Y, or any combination thereof.

15-16. (canceled)

17. The plurality of polynucleotides of claim 1, wherein the polymorphic nucleotide is at a single nucleotide polymorphism (SNP) site.

18. The plurality of polynucleotides of claim 1, wherein the polymorphic nucleotide comprises a plurality of polymorphic nucleotides, for example, nucleotides at a plurality of single nucleotide polymorphism (SNP) sites.

19. The plurality of polynucleotides of claim 1, comprising between about 50 and about 150 polynucleotides (e.g., about 120 polynucleotides) configured to bind to a target sequence on human chromosome 1, e.g., any of target sequences 1-117 as set forth in Table 1 (the Table in FIG. 10), or a complementary or substantially complementary sequence thereof.

20. The plurality of polynucleotides of claim 1, comprising between about 10 and about 50 polynucleotides (e.g., about 40 polynucleotides) configured to bind to a target sequence on human chromosome 2, e.g., any of target sequences 2747-2784 as set forth in Table 1 (the Table in FIG. 10), or a complementary or substantially complementary sequence thereof.

21. The plurality of polynucleotides of claim 1, comprising between about 10 and about 80 polynucleotides (e.g., about 60 polynucleotides) configured to bind to a target sequence on human chromosome 3, e.g., any of target sequences 4072-4126 as set forth in Table 1 (the Table in FIG. 10), or a complementary or substantially complementary sequence thereof.

22. The plurality of polynucleotides of claim 1, comprising between about 10 and about 80 polynucleotides (e.g., about 50 polynucleotides) configured to bind to a target sequence on human chromosome 4, e.g., any of target sequences 4127-4171 as set forth in Table 1 (the Table in FIG. 10), or a complementary or substantially complementary sequence thereof.

23. The plurality of polynucleotides of claim 1, comprising between about 10 and about 80 polynucleotides (e.g., about 40 polynucleotides) configured to bind to a target sequence on human chromosome 9, e.g., any of target sequences 4172-4212 as set forth in Table 1 (the Table in FIG. 10), or a complementary or substantially complementary sequence thereof.

24. The plurality of polynucleotides of claim 1, comprising between about 100 and about 1,500 polynucleotides (e.g., about 1,200 polynucleotides) configured to bind to a target sequence on human chromosome 13, e.g., any of target sequences 118-1337 as set forth in Table 1 (the Table in FIG. 10), or a complementary or substantially complementary sequence thereof.

25. The plurality of polynucleotides of a claim 1, comprising between about 10 and about 150 polynucleotides (e.g., about 100 polynucleotides) configured to bind to a target sequence on human chromosome 15, e.g., any of target sequences 1338-1444 as set forth in Table 1 (the Table in FIG. 10), or a complementary or substantially complementary sequence thereof.

26. The plurality of polynucleotides of claim 1, comprising between about 100 and about 1,500 polynucleotides (e.g., about 1,200 polynucleotides) configured to bind to a target sequence on human chromosome 18, e.g., any of target sequences 1445-2681 as set forth in Table 1 (the Table in FIG. 10), or a complementary or substantially complementary sequence thereof.

27. The plurality of polynucleotides of claim 1, comprising between about 10 and about 100 polynucleotides (e.g., about 60 polynucleotides) configured to bind to a target sequence on human chromosome 19, e.g., any of target sequences 2682-2746 as set forth in Table 1 (the Table in FIG. 10), or a complementary or substantially complementary sequence thereof.

28. The plurality of polynucleotides of claim 1, comprising between about 100 and about 1,500 polynucleotides (e.g., about 1,200 polynucleotides) configured to bind to a target sequence human chromosome 21, e.g., any of target sequences 2785-3995 as set forth in Table 1 (the Table in FIG. 10), or a complementary or substantially complementary sequence thereof.

29. The plurality of polynucleotides of claim 1, comprising between about 10 and about 120 polynucleotides (e.g., about 70 polynucleotides) configured to bind to a target sequence on human chromosome 22, e.g., any of target sequences 3996-4071 as set forth in Table 1 (the Table in FIG. 10), or a complementary or substantially complementary sequence thereof.

30. The plurality of polynucleotides of claim 1, comprising between about 100 and about 500 polynucleotides (e.g., about 300 polynucleotides) configured to bind to a target sequence on human chromosome X, e.g., any of target sequences 4213-4462 as set forth in Table 1 (the Table in FIG. 10), or a complementary or substantially complementary sequence thereof.

31. The plurality of polynucleotides of claim 1, comprising between about 300 and about 800 polynucleotides (e.g., about 500 polynucleotides) configured to bind to a target sequence on human chromosome Y, e.g., any of target sequences 4463-4962 as set forth in Table 1 (the Table in FIG. 10), or a complementary or substantially complementary sequence thereof.

32. The plurality of polynucleotides of claim 1, comprising between about 300 and about 4,500 polynucleotides (e.g., about 3,600 polynucleotides) configured to bind to target sequences on human chromosomes 13, 18, and 21.

33-34. (canceled)

35. A method for analyzing a fetal genetic information, e.g., fetal fraction, a chromosome abnormality such as trisomy, sex determination and/or prenatal paternity test, comprising:
a) contacting a sample from a female subject with the plurality of polynucleotides of claim 1; and
wherein nucleic acid sequence information of the sample is obtained, which indicates a fetal genetic information.

36-40. (canceled)

61. A kit for analyzing a fetal genetic information, e.g., fetal fraction, a chromosome abnormality such as trisomy, sex determination and/or prenatal paternity test, comprising a plurality of polynucleotides of claim 1.

62-73. (canceled)
